(19) United States
(12) Patent Application Publication
(10) Pub. No.: US 2025/0262329 A1
(43) Pub. Date: Aug. 21, 2025

(54) TREATMENT OF HEART DISEASE BY DISRUPTION OF THE ANCHORING OF PP2A

(71) Applicants: University of Miami, Miami, FL (US); The Board of Trustees of the Leland Stanford Junior University, Stanford, CA (US)

(72) Inventors: Michael S. KAPILOFF, Los Altos, CA (US); Jinliang LI, Palo Alto, CA (US)

(73) Assignees: University of Miami, Miami, FL (US); The Board of Trustees of the Leland Stanford Junior University, Stanford, CA (US)

(21) Appl. No.: 19/044,158

(22) Filed: Feb. 3, 2025

Related U.S. Application Data
(63) Continuation of application No. 18/420,397, filed on Jan. 23, 2024, now abandoned.
Application No. 18/420,397 is a continuation of application No. 16/818,771, filed on Mar. 13, 2020, now Pat. No. 11,938,198.
(60) Provisional application No. 62/848,156, filed on May 15, 2019.

(51) Int. Cl. A61K 48/00; A61K 35/761; A61P 9/04

(52) U.S. Cl. CPC A61K 48/0066; A61K 35/761; A61P 9/04

(57) ABSTRACT

The present invention provides a method of treating heart failure with reduced ejection fraction, by administering to a patient at risk of such damage, a pharmaceutically effective amount of a composition which inhibits the anchoring of PP2A to mAKAPβ. This composition is preferably in the form of a viral based gene therapy vector that encodes a fragment of mAKAPβ to which PP2A binds.

### CROSS-REFERENCE TO RELATED APPLICATIONS

This application is a continuation of U.S. patent application Ser. No. 18/420,397 filed Jan. 23, 2024 which claims priority to U.S. patent application Ser. No. 16/818,771 filed Mar. 13, 2020, which claims priority to U.S. Provisional Patent Application Ser. No. 62/848,156, filed May 15, 2019, which is hereby incorporated by reference in its entirety and this application incorporates by reference in their entireties U.S. patent application Ser. No. 14/821,082, filed Aug. 7, 2015, now U.S. Pat. No. 9,937,228, issued Apr. 10, 2018, U.S. patent application Ser. No. 14/213,583, filed on Mar. 14, 2014, now U.S. Pat. No. 9,132,174, issued on Sep. 15, 2015, U.S. patent application Ser. No. 16/028,004, filed Jul. 5, 2018, U.S. Provisional Application No. 61/798,268, filed Mar. 15, 2013, and U.S. Provisional Application 62/529,224, filed Jul. 6, 2017.

The instant application contains a Sequence Listing, which has been submitted electronically via EFS-Web in XML file format and is hereby incorporated by reference in its entirety. Said XML copy, created on Jan. 19, 2024, is named 65274_2US02_SL.xml and is 126,514 bytes in size.

### STATEMENT OF GOVERNMENTAL SUPPORT

This invention was made with Government support under contract RO1 HL 075398 and HL126825 awarded by the National Institutes of Health. The Government has certain rights in this invention.

### BACKGROUND OF THE INVENTION

In response to chronic stress, the heart's main compensatory mechanism is myocyte hypertrophy, a non-mitotic increase in volume of the contractile cells (Hill and Olson 2008). The adult mammalian myocyte is roughly cylindrical and can grow either in width or length. Because myocytes contribute the vast majority of the myocardial mass of the heart (Jugdutt 2003), concentric and eccentric hypertrophy of the cardiac myocyte result in thickening of heart chamber walls and dilation of the chambers, respectively. In theory, “concentric” myocyte growth in width involving parallel assembly of sarcomeres reduces ventricular wall stress (Law of LaPlace), while “eccentric” lengthwise myocyte growth involving serial assembly of sarcomeres may accommodate greater ventricular volumes without stretching individual sarcomeres beyond the optimum length for contraction (length-tension relationship) (Grossman, Jones, and McLaurin 1975). While the left ventricle will undergo relatively symmetric hypertrophy in response to physiologic stress such as pregnancy or exercise training, concentric ventricular hypertrophy is the predominant initial response to the increased systolic wall stress present in pressure overload diseases such as hypertension or aortic stenosis. Eccentric ventricular hypertrophy predominates during states of volume overload such as occurs following myocardial infarction, as well as during the transition from concentric hypertrophy to the dilated heart in Heart Failure with Reduced Ejection Fraction (HFrEF) in some forms of cardiovascular disease, including diseases mainly characterized by pressure overload. Concentric and eccentric hypertrophy are also present in inherited hypertrophic and dilated cardiomyopathies, respectively.

At the cellular level, cardiac myocyte hypertrophy occurs as the result of an increase in protein synthesis and in the size and organization of sarcomeres within individual myocytes. For a more thorough review of cardiac remodeling and hypertrophy, see Kehat (2010) and Hill (2008), each herein incorporated by reference in their entirety. The prevailing view is that cardiac hypertrophy plays a major role in the development of heart failure. Traditional routes of treating heart failure include afterload reduction, blockage of beta-adrenergic receptors (β-ARs) and use of mechanical support devices in afflicted patients. However, the art is in need of additional mechanisms of preventing or treating pathological cardiac hypertrophy.

Research suggests that mechanisms that induce “compensatory” concentric hypertrophy early in pressure-overload related heart disease predispose the heart to later systolic dysfunction and eventual failure (Schiattarella and Hill 2015). In this regard, results show that targeting of RSK3-mAKAPβ complexes will attenuate cardiac remodeling due to pressure overload and prevent heart failure (Kritzer et al. 2014; Li, Kritzer, et al. 2013). Accordingly, inhibition of signaling pathways that induce remodeling, including concentric hypertrophy, may be desirable early in pressure overload disease. However, the question remained whether efforts to maintain signals that may promote concentric hypertrophy and oppose eccentric hypertrophy would preserve cardiac volumes and contractility when initiated when the heart is at a stage in the disease process characterized by the eccentric growth and ventricular dilatation leading to HFrEF, whether late in pressure overload-related disease or throughout the progression of volume overload-related disease. Further, it is unknown whether the enhancement of concentric myocyte hypertrophy and/or the inhibition of eccentric myocyte hypertrophy in familial dilated cardiomyopathy may be beneficial.

### AKAPs and Cardiac Remodeling

Ventricular myocyte hypertrophy is the primary compensatory mechanism whereby the myocardium reduces ventricular wall tension when submitted to stress because of myocardial infarction, hypertension, and congenital heart disease or neurohumoral activation. It is associated with a nonmitotic growth of cardiomyocytes, increased myofibrillar organization, and upregulation of specific subsets of “fetal” genes that are normally expressed during embryonic life (Frey 2004, Hill 2008). The concomitant aberrant cardiac contractility, Ca²⁺ handling, and myocardial energetics are associated with maladaptive changes that include interstitial fibrosis and cardiomyocyte death and increase the risk of developing heart failure and malignant arrhythmia (Cappola 2008, Hill 2008). Together, these adaptations contribute to both systolic and diastolic dysfunction that are present in different proportions depending upon the underlying disease (Sharma and Kass 2014). Pathological remodeling of the myocyte is regulated by a complex intracellular signaling network that includes mitogen-activated protein kinase (MAPK), cyclic nucleotide, Ca²⁺, hypoxia, and phosphoinositide-dependent signaling pathways (Heineke and Molkentin 2006).

Increased in prevalence by risk factors such as smoking and obesity, in the United States, heart failure affects 6.2 million adults, and each year ˜1,000,000 new adult cases are diagnosed (Benjamin et al. 2019). The prevalence and incidence of heart failure are increasing, mainly because of increasing life span, but also because of the increased prevalence of risk factors (hypertension, diabetes, dyslipidemia, and obesity) and improved survival rates from other types of cardiovascular disease (myocardial infarction [MI] and arrhythmias) (Heidenreich et al. 2013). First-line therapy for patients with heart failure includes angiotensin-converting enzyme (ACE) inhibitors and β-adrenergic receptor blockers (β-blockers) that can improve the survival and quality of life of such patients, as well as reduce mortality for those with left ventricular dysfunction (Group 1987). Subsequent or alternative therapies include aldosterone and angiotensin II receptor blockers, neprilysin inhibitors, loop and thiazide diuretics, vasodilators, and I_{f} current blockers, as well as device-based therapies (Ponikowski et al. 2016). Nevertheless, the 5-year mortality for symptomatic heart failure remains ˜50%, including >40% mortality for those post-MI (Heidenreich et al. 2013; Gerber et al. 2016).

Cardiac hypertrophy can be induced by a variety of neuro-humoral, paracrine, and autocrine stimuli, which activate several receptor families including G protein-coupled receptors, cytokine receptors, and growth factor tyrosine kinase receptors (Brown 2006, Frey 2004). In this context, it is becoming increasingly clear that A-kinase anchoring proteins (AKAPs) can assemble multiprotein complexes that integrate hypertrophic pathways emanating from these receptors. In particular, recent studies have now identified anchoring proteins including mAKAP, AKAP-Lbc, and D-AKAP1 that serve as scaffold proteins and play a central role in organizing and modulating hypertrophic pathways activated by stress signals.

As the organizers of “nodes” in the intracellular signaling network, scaffold proteins are of interest as potential therapeutic targets (Negro, Dodge-Kafka, and Kapiloff 2008). In cells, scaffold proteins can organize multimolecular complexes called “signalosomes,” constituting an important mechanism responsible for specificity and efficacy in intracellular signal transduction (Scott and Pawson 2009). Firstly, many signaling enzymes have broad substrate specificity. Scaffold proteins can co-localize these pleiotropic enzymes with individual substrates, selectively enhancing the catalysis of substrates and providing a degree of specificity not intrinsic to the enzyme's active site (Scott and Pawson 2009). Secondly, some signaling enzymes are low in abundance. Scaffold proteins can co-localize a rare enzyme with its substrate, making signaling kinetically favorable. Thirdly, since many scaffolds are multivalent, scaffold binding can orchestrate the co-regulation by multiple enzymes of individual substrate effectors. Muscle A-kinase anchoring protein (mAKAP, a.k.a. AKAP6) is a large scaffold expressed in cardiac and skeletal myocytes and neurons that binds both signaling enzymes such as protein kinase A (PKA) and the Ca²⁺/calmodulin-dependent phosphatase Calcineurin (CaN) that have broad substrate specificity and signaling enzymes such as p90 ribosomal S6 kinase 3 (RSK3) that is remarkably low in abundance (FIG. 1) (Wang et al. 2015; Pare, Easlick, et al. 2005; Michel et al. 2005a; Kapiloff et al. 1999b). mAKAPβ is the alternatively-spliced isoform expressed in myocytes, in which cells it is localized to the outer nuclear membrane by binding the integral membrane protein nesprin-1α (Pare, Easlick, et al. 2005).

Consistent with its role as a scaffold protein for stress-related signaling molecules in the cardiac myocyte, depletion of mAKAPβ in rat neonatal ventricular myocytes in vitro inhibited hypertrophy induced by α-adrenergic, β-adrenergic, endothelin-1, angiotensin II, and leucine inhibitor factor/gp130 receptor signaling (Zhang et al. 2011; Pare, Bauman, et al. 2005; Dodge-Kafka et al. 2005; Guo et al. 2015). In vivo, along with attenuating hypertrophy induced by short-term pressure overload and chronic β-adrenergic stimulation, mAKAP gene targeting in the mouse inhibited the development of heart failure following long-term pressure overload, conferring a survival benefit (Kritzer et al. 2014). Specifically, mAKAP gene deletion in the mAKAP^{fl/fl}; Tg(Myh6-cre/Esr1*), tamoxifen-inducible, conditional knock-out mouse reduced left ventricular hypertrophy, while greatly inhibiting myocyte apoptosis, and interstitial fibrosis, left atrial hypertrophy, and pulmonary edema (wet lung weight) due to transverse aortic constriction for 16 weeks (Kritzer et al. 2014).

mAKAP gene targeting is also beneficial following myocardial infarction (Kapiloff, unpublished observations). Permanent ligation of the left anterior descending coronary artery (LAD) in the mouse results in myocardial infarction, including extensive myocyte death, scar formation, and subsequent left ventricular (LV) remodeling. Four weeks following LAD ligation, mAKAP conditional knock-out mouse had preserved LV dimensions and function when compared to infarcted control cohorts. mAKAP conditional knock-out mice had preserved LV ejection fraction and indexed atrial weight compared to controls, while displaying a remarkable decrease in infarct size.

Introduction to mAKAP and Cardiac Remodeling

mAKAP was originally identified in a cDNA library screen for new cAMP-dependent protein kinase (PKA) regulatory-subunit (R-subunit) binding proteins, i.e. A-kinase anchoring proteins or AKAPs (Mccartney et al. 1995). mAKAP was initially named “AKAP100” for the size of the protein encoded by the original cDNA fragment (Mccartney et al. 1995). Subsequently, the full-length mRNA sequence for mAKAPα, the alternatively-spliced isoform of mAKAP expressed in neurons, was defined, revealing that wildtype mAKAPα is a 255 kDA scaffold (Kapiloff et al. 1999b). The sequence for mAKAPβ, the 230 kDa alternatively-spliced isoform of mAKAP expressed in striated myocytes, was later obtained, showing that when expressed in heart or skeletal muscle, mAKAP is translated from an internal start site corresponding to mAKAPα residue Met-245 (Michel et al. 2005a).

mAKAP is localized to the nuclear envelope both in neurons and striated cardiac and skeletal myocytes (FIG. 6), the three cell types in which mAKAP is clearly expressed (Kapiloff et al. 1999b; Pare, Easlick, et al. 2005; Michel et al. 2005a). mAKAP is not a transmembrane domain protein and contains three spectrin-like repeat regions (residues 772-1187) that confer its localization (Kapiloff et al. 1999b). Binding of mAKAP's third spectrin repeat (residues 1074-1187) by the outer nuclear membrane protein nesprin-1α is both necessary and sufficient for mAKAP nuclear membrane localization, at least in myocytes and when expressed in heterologous cells (Pare, Easlick, et al. 2005). Nesprin-1α may also be present on the inner nuclear envelope where it might bind A-type lamins and emerin. Interestingly, mutations in lamin A/C, emerin, and nesprin-1α have been associated with Emery-Dreyfuss muscular dystrophy, as well as other forms of cardiomyopathy (Bonne et al. 1999; Fatkin et al. 1999; Muchir et al. 2000; Bione et al. 1994; Zhang et al. 2007). However, no disease-causing mutations have yet been identified in the human mAKAP gene, and mAKAPβ knock-out in the mouse heart early in development does not induce cardiomyopathy (Kritzer et al. 2014). Besides binding nesprin-1α, mAKAPβ also binds phospholipase Cε (PLCε) through mAKAP's first spectrin repeat, potentially strengthening its association with the nuclear envelope (Zhang et al. 2011). There were early reports of mAKAPβ being present on the sarcoplasmic reticulum (Mccartney et al. 1995; Marx et al. 2000; Yang et al. 1998), but these findings have been called into question due to technical issues including antibody specificity (Kapiloff, Jackson, and Airhart 2001; Kapiloff et al. 1999b).

Besides PKA, PLCε and nesprin-1α, mAKAPβ binds a wide variety of proteins important for myocyte stress responses: adenylyl cyclase type 5 (AC5), exchange protein activated by cAMP-1 (Epac1), cAMP-specific phosphodiesterase type 4D3 (PDE4D3), MEK5 and ERK5 MAP-kinases, 3-phosphoinositide-dependent protein kinase-1 (PDK1), p90 ribosomal S6 kinases 3 (RSK3), protein kinase Cε (PKCε), protein kinase D (PKD1, PKCμ), the protein phosphatases calcineurin (CaN) Aβ and PP2A, the type 2 ryanodine receptor (RyR2), the sodium/calcium exchanger NCX1, ubiquitin E3-ligases involved in HIF1α regulation, and myopodin (Pare, Bauman, et al. 2005; Pare, Easlick, et al. 2005; Dodge-Kafka et al. 2005; Marx et al. 2000; Kapiloff, Jackson, and Airhart 2001; Michel et al. 2005a; Li et al.; Wong et al. 2008; Zhang et al. 2011; Dodge-Kafka and Kapiloff 2006; Vargas et al. 2012; Faul et al. 2007; Schulze et al. 2003; Kapiloff et al. 2009; Zhang et al. 2013). Bound to mAKAPβ, these signaling molecules co-regulate the transcription factors hypoxia-inducible factor 1α (HIF1α), myocyte enhancer factor-2 (MEF2), and nuclear factor of activated T-cell (NFATc) transcription factors, as well as type II histone deacetylases (FIG. 7) (Kritzer et al. 2014; Li, Vargas, et al. 2013; Li et al. 2010; Wong et al. 2008; Li et al. 2019; Dodge-Kafka et al. 2018). Some of these molecules are bound directly and some indirectly, some constitutively and some in a regulated manner. Thus, it is likely that the composition of mAKAPβ signalosomes depends upon the underlying state of the myocyte. As research continues on mAKAPβ, the list of its binding partners grows, confirming its hypothesized role as an important orchestrator of signaling pathways required for remodeling. Most of what is known about mAKAPβ is based upon work using cultured neonatal rat ventricular myocytes, in which mAKAPβ was early on recognized to be required for the induction of hypertrophy by a variety of upstream receptors, including α- and β-adrenergic and cytokine receptors (Pare, Bauman, et al. 2005; Dodge-Kafka et al. 2005). However, recently, the phenotype of a conditional, cardiac-myocyte specific mAKAPβ knock-out mouse has been published confirming the centrality of mAKAPβ to remodeling (Kritzer et al. 2014). There are various upstream inputs, downstream effectors (outputs), and integrative circuitry within mAKAPβ signalosomes that impact pathological remodeling of the heart.

mAKAPf3—a Prototypical A-Kinase Anchoring Protein

Like most AKAPs, mAKAP contains an amphipathic helix (residues 2055-2072) responsible for binding PKA (Kapiloff et al. 1999b; Kritzer et al. 2012). PKA is a heterotetramer of two R-subunits and two catalytic C-subunits, in the configuration C-R-R-C. Within the holoenzyme, the N-terminal docking and dimerization domains of the PKA R-subunits form a X-type, antiparallel four-helix bundle (Newlon et al. 1999). This bundle contains a hydrophobic groove that accommodates the hydrophobic face of the AKAP amphipathic helix. mAKAPβ binds selectively type II PKA (that contains RII subunits) with high affinity (K_{D}=119 nM) (Zakhary et al. 2000). Interestingly, PKA-mAKAPβ binding is increased 16-fold following RIIα autophosphorylation (Zakhary et al. 2000), potentially affecting PKA-mAKAPβ binding in states of altered β-adrenergic signaling. Besides mAKAPβ, there are over a dozen other AKAPs expressed in the myocyte, each with its own distinct localization and sets of binding partners (Kritzer et al. 2014). Remarkably, mAKAP is one of the rarest AKAPs in the myocyte, such that loss of mAKAP does not even affect the localization of perinuclear PKA (Kapiloff, unpublished observations). Despite the low level of expression of the scaffold, replacement in myocytes of endogenous mAKAPβ with a full-length mAKAPβ mutant that cannot bind PKA is sufficient to inhibit the induction of myocyte hypertrophy (Pare, Bauman, et al. 2005). Thus, mAKAPβ signalosomes serve as an example of both how finely PKA signaling may be compartmentalized even on an individual organelle and how the level of expression of a protein or a protein complex is not necessarily indicative of the functional significance of that protein.

mAKAPβ is remarkable because it binds not only effectors for cAMP signaling, but also enzymes responsible for cAMP synthesis and degradation (Kapiloff et al. 2009; Dodge et al. 2001). The synthesis of cAMP from ATP is catalyzed by adenylyl cyclases (AC), while cAMP metabolism to 5′AMP is catalyzed by phosphodiesterases (PDE). The differential association of ACs and PDEs with AKAPs contributes to cAMP compartmentation in cells, providing both for local activation of cAMP effectors and regulation of local cAMP levels by unique regulatory feedback and feedforward loops (Scott, Dessauer, and Tasken 2013). mAKAP is capable of binding both AC2 and AC5, but AC5 appears to be the relevant mAKAPβ-binding partner in the heart (Kapiloff et al. 2009). The N-terminal, C1 and C2 domains of AC5 bind directly to a unique N-terminal site on mAKAPβ (residues 275-340). AC5 activity is inhibited by PKA feedback phosphorylation that in cells is facilitated by mAKAPβ complex formation (Kapiloff et al. 2009). This negative feedback appears to be physiologically relevant to the maintenance of basal cAMP signaling. When the tethering of AC5 to mAKAPβ is inhibited by a competitive peptide comprising the mAKAP AC5-binding domain, both the cAMP content and size of myocytes were increased in the absence of hypertrophic stimulus (Kapiloff et al. 2009).

mAKAP was the first AKAP shown to bind a PDE (Dodge et al. 2001). A site within mAKAP 1286-1831 binds the unique N-terminal domain of PDE4D3. Phosphorylation of PDE4D3 serine residues 13 and 54 results in increased binding to the scaffold and increased PDE catalytic activity, respectively (Dodge et al. 2001; Sette and Conti 1996; Carlisle Michel et al. 2004). Because increased PDE4D3 activity accelerates cAMP degradation, PKA and PDE4D3 constitute a negative feedback loop that can modulate local cAMP levels and PKA activity (Dodge et al. 2001). PDE4D3 bound to mAKAP serves not only as a PDE, but also as an adapter protein recruiting the MAPKs MEK5 and ERK5 and the cAMP-dependent, Rap1-guanine nucleotide exchange factor Epac1 to the scaffold (Dodge-Kafka et al. 2005). Activation of MEK5 and ERK5 by upstream signals results in PDE4D3 phosphorylation on Ser-579, inhibiting the PDE and promoting cAMP accumulation and PKA activation (Dodge-Kafka et al. 2005; Hoffmann et al. 1999; Mackenzie et al. 2008). Epac1 is less sensitive to cAMP than PKA, such that very high cAMP levels results in the additional activation of mAKAP-associated Epac1. Through Rap1, Epac1 can inhibit ERK5 activity, thus preventing PDE4D3 inhibition by MAPK signaling, resulting presumably in maximal PDE4D3 activity due to concomitant PKA phosphorylation (Dodge-Kafka et al. 2005). As a result, Epac1, ERK5, and PDE4D3 constitute a third negative feedback loop that will attenuate cAMP levels in the vicinity of mAKAP complexes opposing cAMP elevation to extremely high levels.

Additional complexity is afforded by the binding of the serine-threonine phosphatase PP2A to the C-terminus of mAKAP (residues 2083-2319) (Dodge-Kafka et al. 2010). PP2A can catalyze the dephosphorylation of PDE4D3 Ser-54, thereby inhibiting the PDE in the absence of upstream stimulus. PP2A associated with mAKAP complexes contain B56δ B subunits, which are PKA substrates. PKA phosphorylation enhances PP2A catalytic activity (Ahn et al. 2007), such that phosphorylation of B56δ by mAKAP-bound PKA increases PDE4D3 dephosphorylation, inhibiting the PDE. This presumably increases cAMP levels, constituting a positive feedforward loop for the initiation of cAMP signaling. Together with the negative feedback loops based upon AC5 phosphorylation and PDE4D3 regulation by PKA and ERK5, one would predict that cAMP levels at mAKAPβ signalosomes would be tightly controlled by upstream β-adrenergic and MAPK signaling. Signaling upstream of AC5 and ERK5 will promote cAMP signaling that will be initially promoted by PP2A feedforward signaling, while PDE4D3 activation and AC5 inhibition by PKA and Epac1 negative feedback will constrain signaling. Interestingly, Rababa'het al. demonstrated how mAKAP proteins containing non-synonymous polymorphisms differentially bound PKA and PDE4D3 (Rababa'h et al. 2013). The potential for cAMP signaling to be differentially modulated by crosstalk between upstream signaling pathways or by human polymorphisms makes compelling further work in myocytes to show the relevance of this complicated signaling network.

mAKAPβ and MAP-kinase-RSK3 Signaling

The recruitment of ERK5 by PDE4D3 to mAKAPβ complexes was initially shown to be relevant to the local regulation of cAMP through the aforementioned feedback loops (Dodge-Kafka et al. 2005). However, ERK5 was also recognized to be an important inducer of myocyte hypertrophy, preferentially inducing the growth in length (eccentric hypertrophy) of cultured myocytes, while also being important for concentric hypertrophy in vivo due to pressure overload (transverse aortic constriction in the mouse) (Nicol et al. 2001; Kimura et al. 2010). Notably, inhibition by RNA interference (RNAi) of mAKAPβ expression in cultured myocytes inhibited the eccentric growth induced by the interleukin-6-type cytokine leukemia inhibitory factor (LIF) (Dodge-Kafka et al. 2005). A potential effector for mAKAPβ-bound ERK5 was MEF2 transcription factor, as discussed below. However, in both heart and brain, mAKAP bound PDK1, a kinase that together with ERKs (ERK1, 2 or 5) can activate the MAPK effector p90RSK, a kinase also associated with mAKAP (Ranganathan et al. 2006; Michel et al. 2005a). Importantly, binding of PDK1 to mAKAP obviated the requirement for membrane association in RSK activation (Michel et al. 2005a). Taken together, these data suggested that mAKAPβ could orchestrate RSK activation in myocytes in response to upstream MAPK signaling.

p90RSK is a pleiotropic ERK effector that regulates many cellular processes, including cell proliferation, survival, migration, and invasion. RSK activity is increased in myocytes by most hypertrophic stimuli (Anjum and Blenis 2008; Sadoshima et al. 1995). In addition, RSK activity was found to be increased in human end-stage dilated cardiomyopathy heart tissue (Takeishi et al. 2002). RSK family members contain 2 catalytic domains, an N-terminal kinase domain and a C-terminal kinase domain (Anjum and Blenis 2008). The N-terminal kinase domain phosphorylates RSK substrates and is activated by sequential phosphorylation of the C-terminal and N-terminal kinase domain activation loops by ERK and PDK1, respectively, such that PDK1 phosphorylation of the N-terminal domain on Ser-218 is indicative of full activation of the enzyme. There are 4 mammalian RSK family members that are ubiquitously expressed, but only RSK3 binds mAKAPβ (Li, Kritzer, et al. 2013). The unique N-terminal domain of RSK3 (1-30) binds directly mAKAPβ residues 1694-1833, explaining the selective association of that isoform with the scaffold (Li, Kritzer, et al. 2013). Despite the fact that RSK3 is expressed less in myocytes than other RSK family members, neonatal myocyte hypertrophy was found to be attenuated by RSK3 RNAi, inactivation of the RSK3 N-terminal kinase domain, and disruption of RSK3 binding to mAKAP using an anchoring disruptor peptide (Li, Kritzer, et al. 2013). Importantly, RSK3 expression in vivo was required for the induction of cardiac hypertrophy by both pressure overload and catecholamine infusion, as well as for the heart failure associated with a mouse model for familial hypertrophic cardiomyopathy (α-tropomyosin Glu180Gly) (Li, Kritzer, et al. 2013; Passariello et al. 2013). In addition, consistent with the reported role of ERK1/2 MAP-Kinase in selectively inducing concentric hypertrophy (Kehat et al. 2011), RSK3 gene deletion inhibited the concentric hypertrophy induced by Raf1^{L613V} mutation in a mouse model for Noonan Syndrome (Passariello et al. 2016). The recognition that this specific RSK isoform is required for cardiac remodeling makes it a compelling candidate for therapeutic targeting.

mAKAPβ and Phosphatidylinositide Signaling

The cAMP effector Epac1 activates Rap1 at mAKAPβ complexes affecting ERK5 signaling (Dodge-Kafka et al. 2005). In addition, Epac1-Rap1 activates PLCε, a phospholipase whose Ras association domains directly bind the first spectrin repeat-like domain of mAKAPβ (Zhang et al. 2011). Like mAKAPβ, PLCε was required for neonatal myocyte hypertrophy, whether inhibited by RNAi or by displacement from mAKAPβ by expression of competitive binding peptides. In an elegant paper by the Smrcka laboratory, mAKAPβ-bound PLCε has been shown to regulate PKCε and PKD activation through a novel phosphatidylinositol-4-phosphate (PI4P) pathway in which PLCε selectively converts perinuclear PI4P to diacylglycerol and inositol-1,4-bisphosphate (Zhang et al. 2013). PKD1 phosphorylates type II histone deacetylases (HDACs 4/5/7/9) inducing their nuclear export and de-repressing hypertrophic gene expression (Monovich et al. 2010; Xie and Hill 2013). Smrcka and colleagues found that PLCε was required for pressure overload-induced PKD activation, type II HDAC phosphorylation and hypertrophy in vivo (Zhang et al. 2013). Subsequently, mAKAPβ was also found to be is required in vivo for PKD activation and HDAC4 phosphorylation in response to pressure overload (Kritzer et al. 2014). Remarkably, mAKAPβ can form a ternary complex with PKD and HDAC4. Together, these results show how local cAMP signaling can affect the regulation of cardiac gene expression.

Recently it was published that mAKAPβ is a scaffold for HDAC5 in cardiac myocytes, forming signalosomes containing HDAC5, PKD, and PKA (Dodge-Kafka et al. 2018). Inhibition of mAKAPβ expression attenuated the phosphorylation of HDAC5 by PKD and PKA in response to α- and β-adrenergic receptor stimulation, respectively. Importantly, disruption of mAKAPβ-HDAC5 anchoring prevented the induction of HDAC5 nuclear export by α-adrenergic receptor signaling and PKD phosphorylation. In addition, disruption of mAKAPβ-PKA anchoring prevented the inhibition by β-adrenergic receptor stimulation of α-adrenergic-induced HDAC5 nuclear export. Together, these data establish that mAKAPβ signalosomes serve to bidirectionally regulate the nuclear-cytoplasmic localization of class IIa HDACs. Thus, the mAKAPβ scaffold serves as a node in the myocyte regulatory network controlling both the repression and activation of pathological gene expression in health and disease, respectively.

mAKAPβ and Calcium Signaling

Besides cAMP, phosphoinositide and MAP-kinase signaling, mAKAPβ contributes to the orchestration of Ca²⁺-dependent signaling transduction. The second binding partner for mAKAPβ identified was the ryanodine receptor Ca²⁺ release channel (RyR2) responsible for Ca²⁺-induced Ca²⁺ release from intracellular stores (Kapiloff, Jackson, and Airhart 2001; Marx et al. 2000). RyR2 is best known for its role in excitation-contraction coupling, in which bulk Ca²⁺ is released to induce sarcomeric contraction. PKA phosphorylation can potentiate RyR2 currents (Valdivia et al. 1995; Dulhunty et al. 2007; Bers 2006), although the importance of PKA-catalyzed RyR2 phosphorylation to excitation-contraction coupling is highly controversial (Houser 2014; Dobrev and Wehrens 2014). A small fraction of RyR2, presumably located at perinuclear dyads (Escobar et al. 2011), can be immunoprecipitated with mAKAPβ and nesprin-1α antibodies (Pare, Easlick, et al. 2005; Kapiloff, Jackson, and Airhart 2001). mAKAPβ appears to bring together elements of the excitation-contraction coupling machinery and signaling molecules important for regulating nuclear events germane to pathological remodeling. Thus, mAKAPβ complexes may provide one mechanism for matching contractility to the induction of hypertrophy. β-adrenergic stimulation of primary myocyte cultures results in increased PKA phosphorylation of mAKAPβ-associated RyR2 (Pare, Bauman, et al. 2005). PKA-catalyzed RyR2 phosphorylation may potentiate local Ca²⁺ release within the vicinity of mAKAPβ signalosomes during states of elevated sympathetic stimulation.

While it is unlikely that the few mAKAPβ-associated RyR2s could affect overall contractility, a potential target for increased perinuclear Ca²⁺ may be the Ca²⁺/calmodulin-dependent phosphatase calcineurin (CaN) that can bind the scaffold. There are three isoforms of the catalytic subunit for CaN (α,β,γ┐, but only CaNAβ-mAKAPβ complexes have been detected in myocytes (Li et al. 2010). Remarkably, CaNAβ is the CaNA isoform important for the induction of cardiac hypertrophy in vivo, as well as for myocyte survival after ischemia (Bueno et al. 2002; Bueno et al. 2004). CaNAβ binds directly to a unique site within mAKAPβ (residues 1286-1345) (Pare, Bauman, et al. 2005; Li et al. 2010). CaNAβ binding to mAKAPβ is enhanced in cells by adrenergic stimulation and directly by Ca²⁺/calmodulin (Li et al. 2010). Notably, CaNAβ-mAKAPβ binding was required for α-adrenergic-induced neonatal myocyte hypertrophy in vitro (Li et al. 2010).

mAKAPβ and Gene Expression

Among its many substrates, CaN is responsible for the activation of NFATc and MEF2 transcription factors. The NFATc transcription factor family includes four CaN-dependent isoforms that are all expressed in myocytes and that can contribute to the induction of myocyte hypertrophy (Wilkins et al. 2004). In general, NFATc family members are retained in the cytoplasm when heavily phosphorylated on the multiple serine-rich motifs within the N-terminal regulatory domain. NFATc translocates into the nucleus when these motifs are dephosphorylated by CaN. Multiple NFATc family members can bind mAKAPβ, and binding to mAKAPβ was required for CaN-dependent dephosphorylation of NFATc3 in myocytes (Li et al. 2010). Accordingly, mAKAPβ expression was also required for NFAT nuclear translocation and transcriptional activity in vitro (Li et al. 2010; Pare, Bauman, et al. 2005). These results correlate with recent observations that NFAT-dependent gene expression in vivo was attenuated by mAKAPβ cardiac-myocyte specific knock-out following transverse aortic constriction (Kritzer et al. 2014).

Like NFATc2 and NFATc3, MEF2D is a transcription factor required for cardiac hypertrophy in vivo (Kim et al. 2008; Wilkins et al. 2002; Bourajjaj et al. 2008). MEF2 family members contain a conserved DNA binding domain that includes both a MADS box and a MEF2 homology domain (Potthoff and Olson 2007). The DNA-binding domain of MEF2D binds directly to an N-terminal domain of mAKAP (Vargas et al. 2012; Kim et al. 2008). CaN and MEF2D are important not only in the heart, but also in skeletal muscle (Naya et al. 1999; Naya and Olson 1999; Black and Olson 1998; Friday et al. 2003; Wu et al. 2001). Interference with MEF2-mAKAPβ binding blunted MEF2 transcriptional activity and the expression of endogenous MEF2 target genes in C2C12 skeletal myoblasts (Vargas et al. 2012). In addition, disruption of MEF2-mAKAP complexes attenuated the differentiation of C2C12 myoblasts into myotubes, as evidenced by decreased cell fusion and expression of differentiation markers (Vargas et al. 2012). Remarkably, CaN-MEF2 binding is mAKAPβ-dependent in cardiac myocytes (Li, Vargas, et al. 2013). Accordingly, disruption of CaN-mAKAPβbinding inhibited both MEF2 transcriptional activity in C2C12 cells and cardiac myocyte hypertrophy (Li, Vargas, et al. 2013). Like NFATc2, MEF2D de-phosphorylation in vivo in response to pressure overload was attenuated following mAKAPβ conditional knock-out, correlating with the decreased expression MEF2-target genes, including the expression of atrial natriuretic factor (Kritzer et al. 2014).

The regulation of NFATc, MEF2 and HDAC4 by mAKAPβ in vivo during pressure overload shows the importance of mAKAPβ to stress-regulated gene expression (Kritzer et al. 2014). Published reports show how, at mAKAPβ, NFATc and MEF2 are regulated by CaN, while HDAC4 and HDAC5 are regulated by PKD and PKA (Li, Vargas, et al. 2013; Zhang et al. 2013; Li et al. 2010; Dodge-Kafka et al. 2018). mAKAPβ appears to facilitate the modulation of these gene regulatory proteins by other signaling enzymes. For example, mAKAPβ-associated ERK5 may phosphorylate MEF2, activating the transcription factor (Kato et al. 2000). In addition, PKA can phosphorylate MEF2, affecting its DNA-binding affinity (Wang et al. 2005). On the other hand, the Olson group has proposed that PKA phosphorylation of HDAC4 can inhibit MEF2 activity through the generation of a novel HDAC4 proteolytic fragment (Backs et al. 2011). How the activities of the many mAKAPβ binding partners are ultimately integrated to control gene expression can be investigated both in vitro and in vivo.

Other mAKAPβ Binding Partners

There are other binding partners for mAKAPβ for whom the significance of docking to the scaffold remains poorly characterized, including myopodin and NCX1 (Faul et al. 2007; Schulze et al. 2003). HIF-1α, a transcription factor that regulates systemic responses to hypoxia, also binds mAKAPβ (Wong et al. 2008). Under normoxic conditions, the abundance of HIF-1α in the cell is kept low by ubiquitin-mediated proteasomal degradation. HIF-1α is hydroxylated by a family of oxygen-sensitive dioxygenases called prolyl hydroxylases (PHD1, PHD2, and PHD3) (Ohh et al. 2000). Hydroxylated HIF-1α is subsequently recognized by the von Hippel-Lindau protein (pVHL), which recruits the Elongin C ubiquitin ligase complex to ubiquitinate HIF-1α and to promote its proteasome-dependent degradation (Maxwell et al. 1999). Under hypoxic conditions, PHDs are inactivated, HIF-1α degradation is decreased and HIF-1α accumulates in the nucleus, where it can dimerize with HIF-1β to promote the transcription of target genes. mAKAPβ can assemble a signaling complex containing HIF-1a, PHD, pVHL and the E3 ligase Siah2 (seven in absentia homolog 2) in cultured neonatal myocytes (Wong et al. 2008). Under normoxic conditions, mAKAPβ-anchored PHD and pVHL favor HIF-1α ubiquitination and degradation (Wong et al. 2008). Under hypoxic conditions, however, Siah2 activation induces proteasomal degradation of bound PHD, favoring HIF-1α accumulation (Wong et al. 2008). An mAKAPβ knock-out may affect cardiac myocyte survival after ischemia-reperfusion.

mAKAPf3—a Conductor of the Remodeling Symphony

The above discussion shows how multiple signaling pathways known to be important for cardiac hypertrophy and pathological remodeling are modulated by the binding of key signaling intermediates to the mAKAPβ scaffold. Cardiac myocyte-specific, conditional mAKAP knock-out mouse has been characterized, showing the relevance of mAKAPβ signalosomes in vivo (Kritzer et al. 2014). mAKAPβ was required in cardiac myocytes for the induction of cardiac hypertrophy by transverse aortic constriction and isoproterenol infusion. Most remarkable, however, was the prevention of pathological remodeling, including myocardial apoptosis and interstitial fibrosis, and the preservation of cardiac function in the face of long-term pressure overload, together resulting in a significant increase in mouse survival (Kritzer et al. 2014). These results established mAKAPβ as the first scaffold whose ablation confers a survival benefit in heart disease. Importantly, mAKAPβ did not appear to be necessary for either the development or maintenance of normal adult cardiac function, as the use of a Nkx2-5-directed cre deleter line did not result in an overt phenotype by six months of age (Kritzer et al. 2014). Although mAKAPβ knock-out did attenuate the physiological hypertrophy induced by forced exercise (swimming), the targeting of mAKAPβ complexes in disease remains relevant.

Various strategies for targeting mAKAPβ complexes in humans may be envisioned, including siRNA knock-down of the scaffold. However, a relatively detailed understanding of the structure and function of mAKAPβ signalosomes provides us with additional approaches to targeting these pathways. For example, the expression of peptides targeting key protein-protein interactions involving mAKAPβ has already been shown to be effective in vitro, including anchoring disruptor peptides targeting mAKAPβ-CaNAβ, mAKAPβ-MEF2D, mAKAPβ-PLCε, and mAKAPβ-RSK3 binding (Li, Vargas, et al. 2013; Li, Kritzer, et al. 2013; Vargas et al. 2012; Zhang et al. 2011). A leading cause of death, heart failure is a disease that incurs 50% mortality within 5 years of diagnosis despite modem therapy, at a cost of over $30 billion/year in the USA alone (Go et al. 2014). Many candidates for potential targeting in cardiac disease are pleiotropic, complicating the development of drugs with sufficient specificity in vivo. The specific targeting of mAKAPβ signalosomes provides an opportunity to target relatively rare protein-protein interactions that appear to be dedicated to pathological cardiac remodeling and whose ablation may be promoted without significant side-effects. There is a clear need to develop new effective therapies to treat patients with heart failure, as well as to prevent its development in the context of other cardiovascular diseases such coronary artery disease, hypertension, and valvular disease.

### SUMMARY OF THE INVENTION

The following brief summary is not intended to include all features and aspects of the present invention, nor does it imply that the invention must include all features and aspects discussed in this summary.

The present inventors have discovered methods of treating cardiac pathological processes by inhibiting the signaling properties of individual mAKAP signaling complexes using drugs that target unique protein-protein interactions. Such a therapeutic strategy offers an advantage over classical therapeutic approaches because it allows the selective inhibition of defined cellular responses.

In particular, the present inventors have found that disrupting mAKAP-mediated protein-protein interactions can be used to inhibit the ability of mAKAP to coordinate the activation of enzymes that play a central role in activating key transcription factors that initiate cellular processes leading to pathological cardiac remodeling.

Specifically, the inventors have discovered that inhibiting the binding interaction between PP2A and mAKAPβ can protect the heart from damage leading to heart failure, for example, following myocardial infarction.

Thus, the present invention comprises, in certain aspects a method for protecting the heart from damage, by administering to a patient at risk of such damage, a pharmaceutically effective amount of a composition which inhibits the interaction of PP2A and mAKAPβ.

The invention also relates to a method of treating heart disease, by administering to a patient a pharmaceutically effective amount of a composition which inhibits the interaction of PP2A and mAKAPβ.

The invention also relates to compositions which inhibit the interaction of PP2A and mAKAPβ.

In still other embodiments, the inhibitors include any molecule that inhibits the expression or activity of PP2A and mAKAPβ.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Model for mAKAPβ-regulated, SRF-dependent gene expression. Anchored RSK3 is a Gq-protein coupled receptor-ERK effector that phosphorylates SRF associated with perinuclear mAKAPβ complexes. mAKAPβ-anchored PP2A that can be activated by cAMP-dependent protein kinase A (PKA) opposes SRF phosphorylation. Phosphorylated SRF induces gene expression that promotes concentric hypertrophy.

FIG. 2. Shows the amino acid sequence of human RSK3 (SEQ ID NO: 1).

FIG. 3. Shows the amino acid sequence of rat mAKAP (SEQ ID NO: 2). —Note that within this document, references to mAKAP sequences, whether labelled “mAKAPβ” or “mAKAP” are according to the numbering for the mAKAPα alternatively-spliced form which contains within the entirety of mAKAPβ and is identical to the originally published mAKAP sequence as shown in this figure (Kapiloff 1999, Michel 2005). “mAKAP” is also referred to as “AKAP6” in reference databases and the literature. mAKAPβ starts at residue 245, while mAKAPα starts at residue 1. PP2A binding domain starts at residue 2134.

FIG. 4. Amino acid sequence of rat mAKAP PBD as expressed in AAV vector. Includes N-terminal myc tag (SEQ ID NO: 12).

FIG. 5. Sequence for pscA-TnT-myc-rat mAKAP PBD plasmid used to generate AAV9sc.rat PBD (SEQ ID NOs: 13 and 14).

FIG. 6. mAKAPβ—A Perinuclear Scaffold. A. Top montage: Mouse heart sections (left ventricle) stained for with mAKAP antibody (gray scale panels and green), Hoechst nuclear stain (blue), and wheat germ agglutinin (red, shown in enlarged control image only). Lower left panels are from control, mAKAP knock-out mice. Bar=20 μm. Bottom montage: Adult rat myocyte stained with antibodies to mAKAP (green) and actinin (red). B. mAKAP domain structure. Direct binding partners whose sites have been finely mapped in mAKAPβ are shown. mAKAPβ starts at residue 245 of mAKAPα. Therefore, all binding sites are numbered per mAKAPα. Images are from Kritzer, et al. (Kritzer et al. 2014).

FIG. 7. mAKAPβ Signaling Modules. mAKAPβ binds multiple signaling enzymes and gene regulatory proteins. Modules may be defined that involve cAMP, Ca²⁺, hypoxic, phosphatidylinositide and MAPK signaling. See above for details. In this figure, the mAKAPβ scaffold is presented as a yellow globe sitting on a grey base representing nesprin-1α, on which are assembled the various signaling molecules. Gold cylinders represent nuclear pore complexes inserted in the nuclear envelope.

FIG. 8. An okadaic acid-sensitive phosphatase regulates mAKAP-associated PDE4D3. A, transfected HEK293 cells expressing both mAKAP and PDE4D3 were treated with either 300 μM okadaic Acid (OA) or 500 μM cyclosporine A (CsA) for 30 min before stimulation with 5 μM forskolin (Fsk) for 10 min. The phosphorylation state of PDE4D3 present in mAKAP antibody immunoprecipitates was determined using a antibody specific for phosphorylated PDE4D3 Ser-54 (top panel). Total PDE4D3 (middle panel) and mAKAP (bottom panel) present in mAKAP antibody immunoprecipitates were detected using non-phospho-specific antibodies. Note that in these experiments mAKAP was GFP-tagged and PDE4D3 was VSV and GFP-tagged, resulting in increased molecular weights. n=3 B, PDE activity associated with mAKAP antibody immunoprecipitates prepared as in A was assayed using [³H]cAMP substrate. *p<0.05 compared to untreated cells (bar 1). C, endogenous protein complexes were isolated using control (IgG) or mAKAP-specific antibodies from clarified adult rat heart extracts (500 μg total protein). PDE activity associated with the immunoprecipitates was assayed in the presence of 10 nM OA or 50 nM PKI. n=3; *p<0.05.

FIG. 9. The protein phosphatase PP2A is associated with the mAKAP scaffold in adult rat heart. A, phosphatase activity associated with protein complexes immunoprecipitated using mAKAP antibody from adult rat heart extracts (500 μg total protein) was assayed using ³²P-labelled histone substrate in the absence or presence of 30 nM PP2A Inhibitor I (Li, Makkinje, and Damuni 1996) and 100 nM PKA-phosphorylated PP1 Inhibitor-1 (Endo et al. 1996). n=3. *p<0.05. B & C, protein complexes were isolated from adult rat heart extracts (2 mg total protein) using control (IgG) or mAKAP-specific antibody. PP2A (panel B) and PP1 (panel C) catalytic subunits in extracts (80 μg) and immunoprecipitates (25% loaded) were detected by immunoblotting. n=3.

FIG. 10. PP2A binds a C-terminal mAKAP domain. A, schematic of mAKAP domains and GFP- and myc-tagged mAKAP proteins used in this paper. mAKAP fragments containing rat and human protein are drawn in black and grey, respectively. Hatched bars indicate the three spectrin repeat domains responsible for nuclear envelope targeting in myocytes (Kapiloff et al. 1999a). Binding sites are indicated for proteins known to bind mAKAP directly, including 3-phosphoinositide-dependent kinase-1 (PDK1, mAKAP residues 227-232) (Michel et al. 2005b), nesprin-1α (1074-1187) (Pare, Easlick, et al. 2005), ryanodine receptor (RyR2, 1217-1242) (Marx et al. 2000), PP2B (1286-1345) (Li et al. 2009), PDE4D3 (1285-1833) (Dodge et al. 2001), and PKA (2055-2072) (Kapiloff et al. 1999a). The stippled bar marks the PP2A binding site. The first and last residues of each fragment are indicated. B, purified GST-PP2A A subunit fusion protein was incubated with extracts prepared from HEK293 cells expressing the indicated GFP-mAKAP fusion protein and pulled down using glutathione resin. GFP-mAKAP fragments were detected in the pull-downs (25% loaded, top panel) and the extracts (5% loaded, bottom pane) using a GFP antibody. n=3. C, myc-tagged mAKAP fragments were expressed in HEK293 cells, and phosphatase binding was detected by immunoprecipitation using control (IgG) or myc-tag antibody followed by phosphatase assay using ³²P-labelled histone substrate. n=3. *p<0.05 compared to the other samples. Note that the C-terminal homologous domain of both rat and human mAKAP binds PP2A.

FIG. 11. PP2A association with mAKAP-PDE4D3 complexes is required for inhibition of PDE4D3 phosphorylation. A, HEK293 cells expressing (VSV and GFP-tagged) PDE4D3 and myc-tagged mAKAP 1286-2312 or 1286-2083 lacking the PP2A binding site were treated with 300 μM OA for 30 minutes before stimulation with 5 μM Fsk for 10 minutes. Protein complexes were immunoprecipitated using myc-tag antibody in the presence of phosphatase inhibitors. The phosphorylation state of co-immunoprecipitated PDE4D3 was determined using an antibody specific for phosphorylated PDE4D3 Ser-54 (P-PDE4D3, top panel). Total PDE4D3, myc-mAKAP, and PP2A C-subunit present in the immunoprecipitates were detected using non-phospho-specific antibodies (lower three panels). n=3. B, PDE activity associated with myc-antibody immunoprecipitates isolated from additional cells treated as in A was assayed using [³H]cAMP. n=3. *p<0.05 compared to bar 1.

FIG. 12. mAKAP-bound PP2A contains B568-subunit and is cAMP-activated. A, protein complexes were immunoprecipitated from adult rat heart extracts (500 μg total protein) using control (IgG) or mAKAP-specific antibody as in FIG. 9B and assayed for associated phosphatase activity. As indicated, the immunoprecipitates were pre-incubated with no addition or with 50 μM CPT-cAMP, 10 nM OA, or 50 nM PKI for 5 minutes before addition of [32P]histone substrate. n=3. *p<0.05. B, Endogenous protein complexes were immunoprecipitated from adult heart extract (2 mg total protein) with B56δ and control (IgG) antibodies. mAKAP in 80 μg extract and in the immunoprecipitates (25% loaded) was detected by immunoblot. n=3. C, Flag-tagged B56δ and/or GFP-tagged mAKAP were expressed in HEK293 cells. Protein complexes were immunoprecipitated using a mAKAP antibody. B56δ in the immunoprecipitates (25% loaded) and total extracts (5% loaded) was detected by immunoblotting with a Flag antibody. n=3. D, phosphatase activity associated with mAKAP-antibody immunoprecipitates prepared as in C was assayed using ³²P-labelled histone substrate. n=3. E, HEK293 cells expressing mAKAP and B56δ were treated with 5 μM Fsk and 10 μM IBMX (Fsk/IBMX) for 10 min before immunoprecipitation of protein complexes with mAKAP antibody. Phosphatase activity associated with the immunoprecipitates was assayed using [³²P]histone substrate. n=3. Note that PP2A B56δ and C-subunit binding to mAKAP was not affected by Fsk/IBMX (see FIG. 13 below).

FIG. 13. Phosphorylation of B56δ by PKA increases mAKAP-associated PP2A activity. A, B56δ is phosphorylated on serine residues 53, 68, 81, and 566 by PKA (Ahn et al. 2007). B56δ wildtype or alanine substituted at all four PKA sites (S4A) was co-expressed in HEK293 cells with wildtype mAKAP or a full-length mAKAP mutant lacking the PKA binding site (ΔPKA; FIG. 10A). After stimulation with 5 μM Fsk and 50 μM IBMX, protein complexes were immunoprecipitated with mAKAP antibody, and associated proteins were detected by immunoblotting with B56δ, mAKAP, and PP2A-C antibodies (lower three panels). PKA phosphorylation of B56δ was detected by immunoblotting with a B56δ phospho-Ser-566 specific antibody (P-B56δ, upper panel). n=3. B, Immunoprecipitates prepared as in B were assayed for associated phosphatase activity. n=3. *p<0.05.

FIG. 14. Phosphorylation of B56δ by PKA enhances the dephosphorylation of mAKAP-associated PDE3D3. A, HEK293 cells expressing (GFP-tagged) mAKAP, (VSV- and GFP-tagged) PDE4D3 and either wild-type B56δ or B56δ S4A mutant at the PKA phosphorylation sites were treated as indicated with 300 μM OA for 30 min before stimulation for 10 min with 5 μM Fsk. Protein complexes were immunoprecipitated with mAKAP antibody in the presence of phosphatase inhibitors. The phosphorylation state of PDE4D3 present in the immunoprecipitates was determined using an antibody specific for phosphorylated PDE4D3 Ser-54 (top panel). Total PDE4D3, mAKAP, B56δ and PP2A-C protein present in the immunoprecipitates were detected using non-phospho-specific antibodies (lower four panels). n=3. B, PDE activity associated with protein complexes isolated from additional cells treated as in A was assayed using [³H]cAMP. n=3. *p<0.05 compared to bar 1.

FIG. 15. PKA and PP2A associated with mAKAP complexes coordinately regulate PDE4D3 activity and cAMP degradation. PKA is composed of two regulatory and two catalytic subunits. mAKAP-bound PP2A contains an A, B56δ, and C (catalytic) subunits. A, in unstimulated cells, basal PP2A activity maintains PDE4D3 dephosphorylation, presumably allowing for a more rapid rise in cAMP levels in response to subsequent agonist than if PDE4D3 were phosphorylated and activated. At the same time, basal PDE4D3 activity should maintain low local levels of cAMP, preventing spurious signaling. B, Gₛ-coupled receptor stimulation induces cAMP synthesis, exceeding the rate of cAMP degradation by PDE4D3 and activating mAKAP-bound PKA. PKA phosphorylates and activates both PDE4D3 and PP2A. PDE4D3 activation should limit peak cAMP levels, as well as accelerate the rate of cAMP clearance after GPCR down-regulation. In contrast, PP2A activation opposes PDE4D3 phosphorylation by PKA, attenuating cAMP degradation and contributing to greater, longer lasting cAMP signals.

FIG. 16. Confirmation that PKA-phosphorylated I-1 inhibits PP1 activity. Protein complexes were immunoprecipitated from rat heart extracts with PP1 or control IgG antibody, and associated phosphatase activity was assayed using [³²P]histone substrate in the absence or presence of 100 nM PKA-phosphorylated PP1 Inhibitor-1 (Endo et al. 1996). n=3.

FIG. 17. Distribution of mAKAP and PP2A catalytic subunit in rat neonatal cardiac myocytes. Rat neonatal ventricular myocytes were isolated as previously described (Pare, Easlick, et al. 2005). After treatment with 50 μM phenylephrine for one week to induce myofibrillar organization and mAKAP expression, the cells were fixed and stained with 0.25 μg/ml mouse anti-PP2A-C(green), 0.1 μg/ml OR010 rabbit anti-mAKAP (red) affinity purified antibodies and rhodamine phalloidin (blue in composite image) to show actin myofibrils as previously described (Pare, Easlick, et al. 2005). 4-color Images were acquired on a Zeiss LSM510/UV Confocal Microscope at 400×. Separate PP2A C-subunit and mAKAP images are shown for clarity. PP2A-C subunit was present in a diffuse punctuate pattern in the cytosol, while mAKAP was limited to the location of the nuclear envelope. The presence of PP2A-C subunit staining over the nuclear envelope is consistent with the presence of PP2A-mAKAP complexes (yellow in composite image). Control IgG staining is shown in the right panel. n=3.

FIG. 18. mAKAP Fragments do not bind PP1 in HEK293 cells. mAKAP-GFP fusion proteins were expressed in HEK293 cells and protein complexes were immunoprecipitated with PP1 antibody. Despite robust expression (bottom panels), no mAKAP fusion proteins were precipitated with the PP1 antibody. n=3.

FIG. 19. SRF phosphorylation is regulated by mAKAPβ signalosomes in cardiac myocytes. (A) SRF Domain Structure. Known phosphorylated residues are indicated (Li et al. 2014; Mack 2011; Janknecht et al. 1992). (B) Neonatal rat ventricular myocytes (NRVM) transiently transfected with siRNA and SRE-luciferase and control renilla luciferase plasmids. Normalized luc:rluc ratios are shown. n=3. (C) Co-immunoprecipitation of endogenous complexes from mouse heart extracts. n=3. (D) HA-tagged RSK3 WT or S218A inactive mutant (Li, Kritzer, et al. 2013) and/or myc-mAKAPβ were expressed in COS-7 cells for co-immunoprecipitation assay. n=3. (E) NRVM extracts obtained 2 days after transfection with siRNA+/−10 μM PE. n=3. * vs. control siRNA+PE; † vs. control siRNA+no drug. (F) Adult rat ventricular myocytes (ARVM) infected with adenovirus expressing myc-GFP or myc-GFP-RBD and treated for 1 day with 20 μM PE. n=3. * vs. myc-GFP+PE; † vs. myc-GFP+no drug. (G) NRVM in minimal maintenance media were treated for 1 hour with 1 μM okadaic acid (OA) or 1 μg/ml cyclosporine A (CsA). n=4. * vs. no drug control. (H) NRVM transfected with control or mAKAP siRNA were used for co-immunoprecipitation assay. PP2A holoenzyme contains an A- and C-subunit homodimer core and a scaffolding B-subunit (Dodge-Kafka et al. 2010). PP2A C-subunit (PP2A-C) was detected by immunoblot. n=3. (I) NRVM infected with adenovirus expressing myc-PBD or β-gal before co-immunoprecipitation assay. n=3. (J) ARVM infected with myc-PBD or β-gal adenoviruses and treated for 1 day with 10 μM Iso. n=4. * vs. β-gal+Iso; † vs. β-gal+no drug.

FIG. 20. SRF S¹⁰³ phosphorylation is a determinant of myocyte concentric growth. Adult rat ventricular myocytes (ARVM) were infected with adenovirus and cultured for 24 hours +/−20 μM PE or 10 μM Iso before immunocytochemistry and measurement of cell width and length (maximum dimension parallel or perpendicular to striations; bars=25 μm). (A,B) Myocytes were infected with adenovirus expressing either β-gal (control) or HA-tagged RSK3 and maintained in minimal media. Top: α-actinin—red, nuclei—blue, HA-RSK3—green; bottom HA-RSK3—greyscale. n=4. (C-F). Myocytes were infected with adenovirus expressing SRF WT, S103D, S103A or control virus. Flag-SRF—green, α-actinin—red, nuclei—blue. * vs. no drug for same virus; † vs. control under the same treatment condition; ‡ vs. SRF WT under the same treatment condition. D: n=3; F: n=5. (G,H) Myocytes were infected with adenovirus expressing myc-GFP or myc-GFP-RBD (green). (I,J) Myocytes were infected with adenovirus expressing myc-PBD or j-gal control. (G-J) α-actinin—red, nuclei—blue. * vs. no drug control for same protein; † vs. control protein with same treatment condition. n=4.

FIG. 21. PP2A dephosphorylates SRF S¹⁰³. GST-SRF fusion protein purified from bacterial extracts and on glutathione beads was incubated with purified 0.5 μg RSK3 (Millipore) for 30 minutes before washing twice with PP2A reaction buffer and then incubating for 30 min with 50 ng purified PP2A +/−10 nM okadaic acid.

FIG. 22. AAV9sc.myc-PBD. A. AAV9sc.myc-PBD includes a minigene that expresses the myc-tagged rat PDB peptide (rat mAKAP aa 2134-2314) and a defective right ITR, conferring self-complementarity and presumably decreasing the latency and increasing the efficacy of expression. (Andino et al., 2007). The AAV has the cardiotrophic serotype 9 capsid protein and directs expression of the encoded protein under the control of the cardiac myocyte-specific, chicken troponin T promoter (cTnT). (Prasad et al., 2011) B. Shuttle plasmid for AAV9sc.myc-PBD.

FIG. 23. PBD anchoring disruptor therapy. (A) myc-tagged rat mAKAP PBD (AAV9sc.myc-PBD) and myc-GFP (AAV9sc.GFP) were expressed in mice using a self-complementary AAV9 and the cardiac myocyte-specific chicken troponin T promoter. (Prasad et al., 2011) (B) Timeline for AAV9sc.myc-PBD treatment study shown in C-H. Mice were 8 weeks old at initiation of study. (C) Representative whole heart pictures at endpoint. Bar=5 mm. (D-H) Serial M-mode echocardiography. n: AAV9sc.myc-PBD—8 (green); AAV9sc.GFP—5 (black). * p-value for difference in cohorts at given time point. LV Remodeling Index=Mass÷End-diastolic volume. LVAW; d—left ventricular anterior wall thickness in diastole.

FIG. 24. Nucleotide sequence of human RSK3 (SEQ ID NO: 15).

FIG. 25. Nucleotide sequence of rat mAKAPα mRNA with open reading frame translated (SEQ ID NOs: 2 and 16).

FIG. 26. Nucleotide sequence of human mAKAPβ mRNA with open reading frame translated (SEQ ID NOs: 17 and 18).

FIG. 27. Nucleotide sequence of human mAKAPα mRNA with open reading frame translated (SEQ ID NOs: 19 and 20).

FIG. 28. Amino acid sequence of human mAKAP. mAKAPα starts at residue 1, mAKAPβ at residue 243. PBD in bold (SEQ ID NO: 8).

FIG. 29. Amino acid sequence of human PBD as expressed in AAV (SEQ ID NO: 9).

FIG. 30. Alignment of human and rat PBD amino acid sequences as expressed by AAV species (SEQ ID NOs: 9 and 12). Rat PBD has an N-terminal Myc-tag [EQKLISEEDL, (SEQ ID NO: 21), FIG. 4). The consensus sequence is represented by SEQ ID NO:22 or SEQ ID NO:23.

FIG. 31. Map of human PBD shuttle plasmid.

FIG. 32. Nucleotide sequence of pscAAV-hmAKAP PBD plasmid (SEQ ID NOs: 10 and 11).

FIG. 33. SRF phosphorylation is decreased in dilated hearts. (A-E) Mouse ventricular protein extracts were assayed for phosphorylated and total SRF 5 min (acute pressure overload, n=4,4) or 16 weeks (heart failure, n=15,19) following TAC or sham survival surgery. (A) Representative western blots. (B) Densitometry of top panel in A. (C) After 5 min of pressure overload, RSK3 was immunoprecipitated using N-16 RSK3 specific antibody and detected using OR43 RSK3 antibody and a phospho-specific antibody for RSK3 S²¹⁸ that indicates RSK3 activation. The immunoprecipitation-western assay was validated using RSK3^{-/-} mice (not shown). n=3 for each condition. (D) 16 weeks of pressure overload induced heart failure. M-mode echocardiography for left ventricular (LV) volume in diastole and systole and ejection fraction showed that TAC hearts were dilated and had systolic dysfunction. Measurement of wet lung weight (indexed to tibial length) indicating the presence of pulmonary edema showed that TAC mice were in heart failure. (E) Densitometry of bottom panel in A. (F-H) Left ventricular tissue from human patients (including nonischemic and ischemic cardiomyopathies and non-dilated congenital heart disease and controls) were assayed for SRF S¹⁰³ phosphorylation and segregated by normal (<5.3 cm, n=7) or elevated (>5.3 cm, n=8) left ventricular interior diameter in diastole (LVID; d). Equal loading for blots was confirmed using Ponceau S stain for major protein bands (not shown).

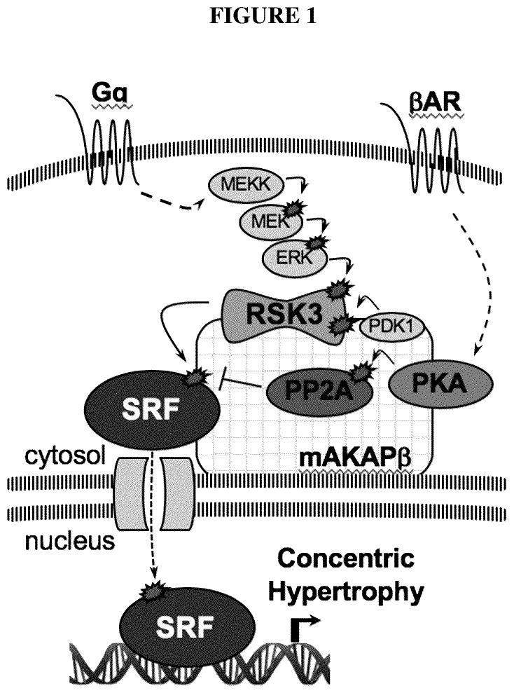

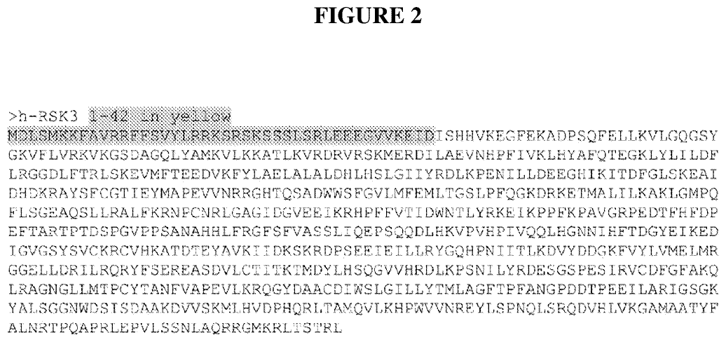

### DETAILED DESCRIPTION OF THE INVENTION

As discussed above, AKAP-based signaling complexes play a central role in regulating physiological and pathological cardiac events. As such, the present inventors have examined inhibiting the signaling properties of individual AKAP signaling complexes using drugs that target unique protein-protein interactions as an approach for limiting cardiac pathological processes. Such a therapeutic strategy offers an advantage over classical therapeutic approaches since it allows the selective inhibition of defined cellular responses.

Anchoring proteins including mAKAP are therapeutic targets for the treatment of cardiac hypertrophy and heart failure. In particular, the present inventors have found that disrupting AKAP-mediated protein-protein interactions can be used to inhibit the ability of mAKAP to coordinate the activation of enzymes that play a central role in activating key transcription factors that initiate the remodeling process leading to cardiac hypertrophy.

One aspect of the current invention is that improved ventricular geometry, i.e. decreased LV internal diameters due to less elongated myocytes and/or increased LV wall thickness due to wider myocytes, will decrease wall stress (Law of LaPlace) and improve systolic function in the heart prone to HFrEF. Demonstration of the prevention of systolic dysfunction has been obtained for a new gene therapy vector based upon expression of a muscle A-kinase anchoring protein (mAKAP, a.k.a. AKAP6)-derived anchoring disruptor peptide for protein phosphatase 2A (PP2A).

As discussed below, the inventors have recently discovered that the transcription factor serum response factor (SRF) is Ser¹⁰³ phosphorylated in the cardiac myocyte by RSK3 at mAKAPβ signalosomes where SRF may in turn be dephosphorylated by protein phosphatase 2A (PP2A) bound to the scaffold. Methods to block the eccentric changes in ventricular morphology that typify end-stage disease and HFrEF are the subject of this invention.

While previously thought to be a constitutive, house-keeping enzyme, it has become apparent that protein phosphatase 2A (PP2A) contributes to the regulation of many phosphorylation events. For example, in the cardiac myocyte, PP2A is involved in the modulation of calcium and MAPK signaling (duBell, Lederer, and Rogers 1996; duBell et al. 2002; Liu and Hofmann 2004). PP2A is a serine/threonine phosphatase that exists as a heterotrimeric complex consisting of a stable, ubiquitously expressed catalytic (PP2A-C) and scaffolding (PP2A-A) subunit heterodimer, and one of 21 known divergent B subunits (Lechward et al. 2001; Wera and Hemmings 1995). PP2A B subunits are grouped into three unrelated families termed B (or PR55), B′ (or B56) and B″ (or PR72) and are proposed to regulate both the catalytic activity and the intracellular targeting of the phosphatase (Virshup 2000). The present inventors have previously shown by reconstitution of mAKAP complexes in heterologous cells that protein phosphatase 2A (PP2A) associated with mAKAP complexes can reverse the activation of PDE4D3 by catalyzing the dephosphorylation of PDE4D3 serine residue 54 (Dodge-Kafka et al. 2010). Mapping studies revealed that a C-terminal mAKAP domain (residues 2085-2319) bound PP2A (Dodge-Kafka et al. 2010). Binding to mAKAP was required for PP2A function on PDE4D3, such that deletion of the C-terminal domain enhanced both baseline and forskolin-stimulated PDE4D3 activity. Interestingly, PP2A holoenzyme associated with mAKAP complexes in the heart contains the PP2A targeting subunit B56δ (Dodge-Kafka et al. 2010). Like PDE4D3, B56δ is a PKA substrate, and PKA phosphorylation of mAKAP-bound B56δ enhanced phosphatase activity 2-fold in the complex. Accordingly, expression of a B56δ mutant that could not be phosphorylated by PKA in heterologous cells with mAKAP resulted in increased PDE4D3 phosphorylation. Taken together, these findings demonstrated that PP2A associated with mAKAP complexes may promote PDE4D3 dephosphorylation, serving to both inhibit PDE4D3 in unstimulated cells and also to mediate a cAMP-induced positive feedback loop following adenylyl cyclase activation and B56δ phosphorylation. Thus PKA-PDE4D3-PP2A-mAKAP complexes exemplify how protein kinases and phosphatases may participate in molecular signaling complexes to dynamically regulate localized intracellular signaling. The relevance to cardiac myocyte function and any potential therapeutic significance were not defined in prior studies (Dodge-Kafka et al. 2010).

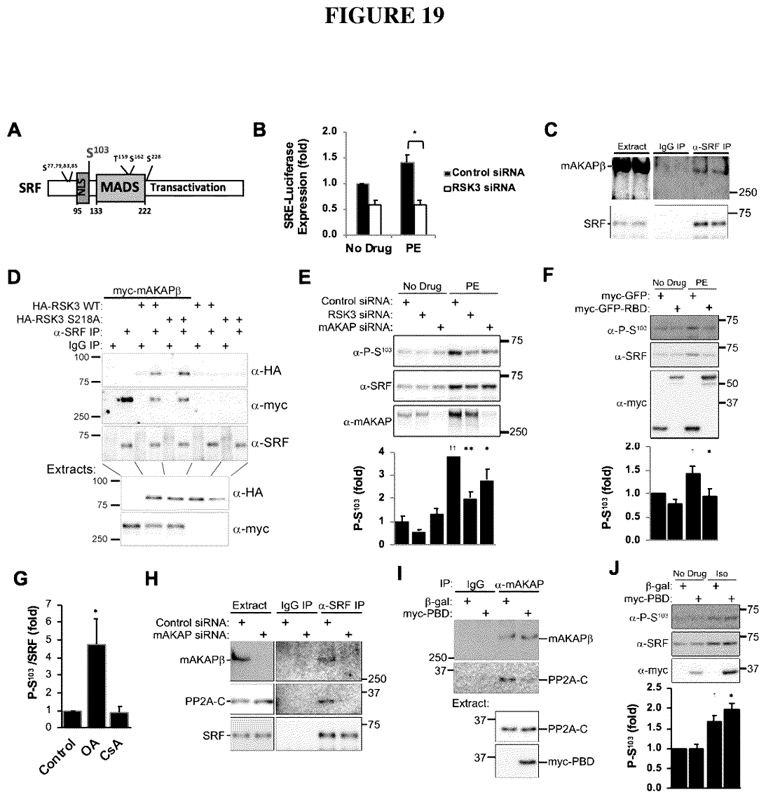

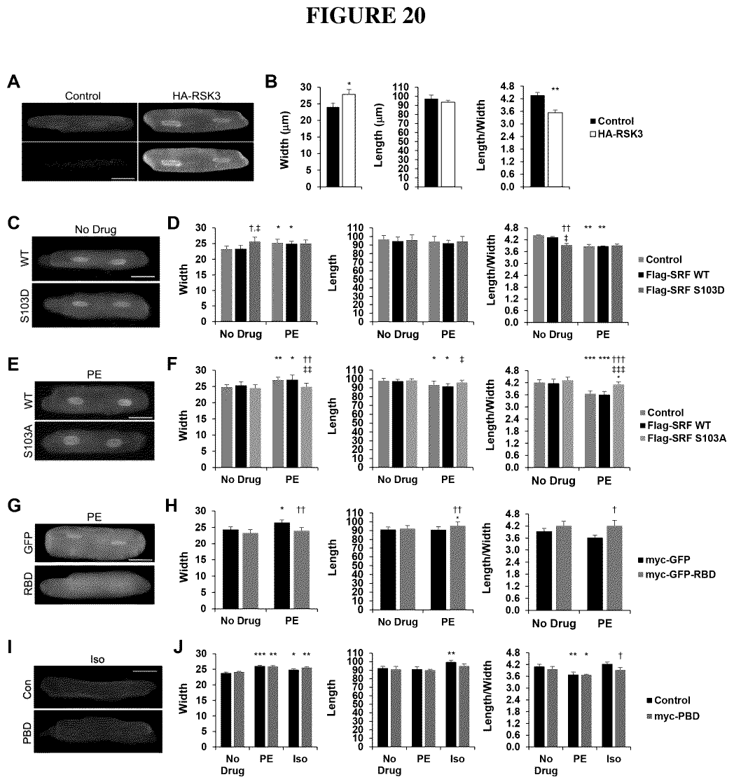

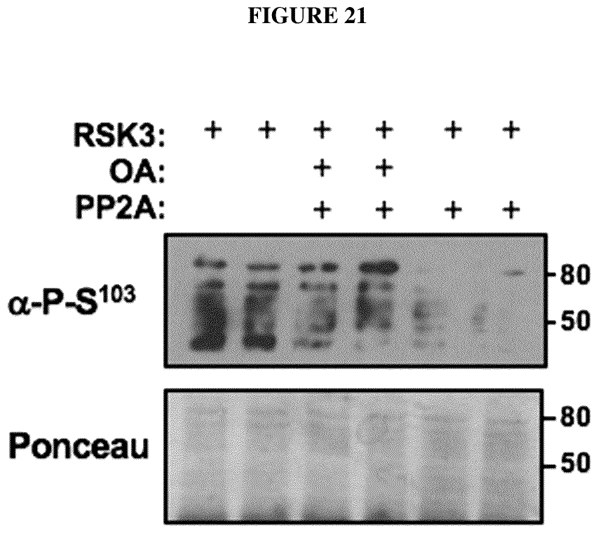

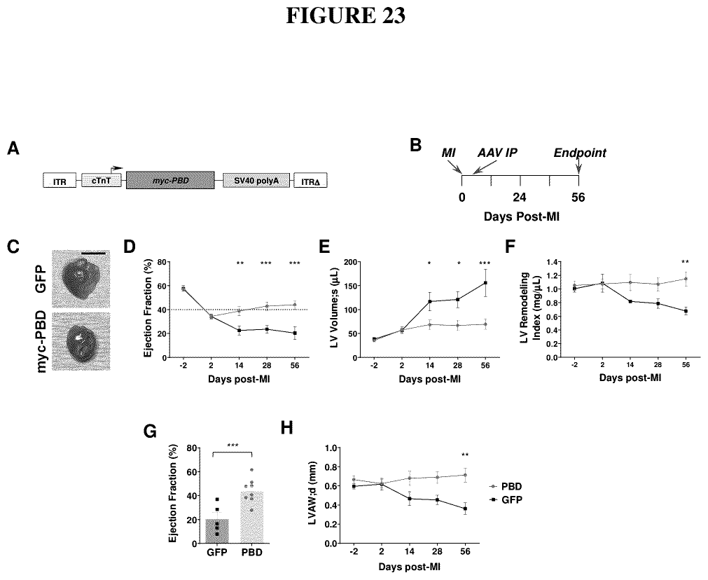

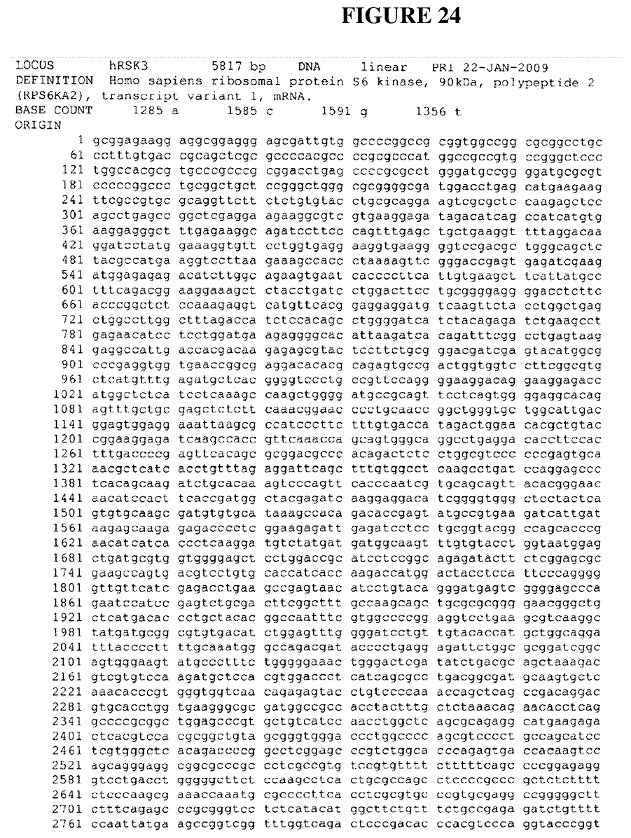

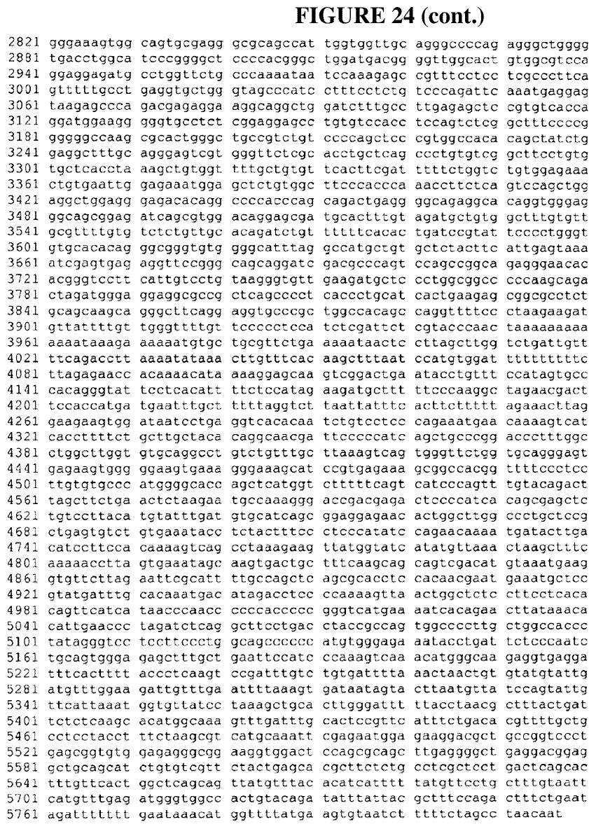

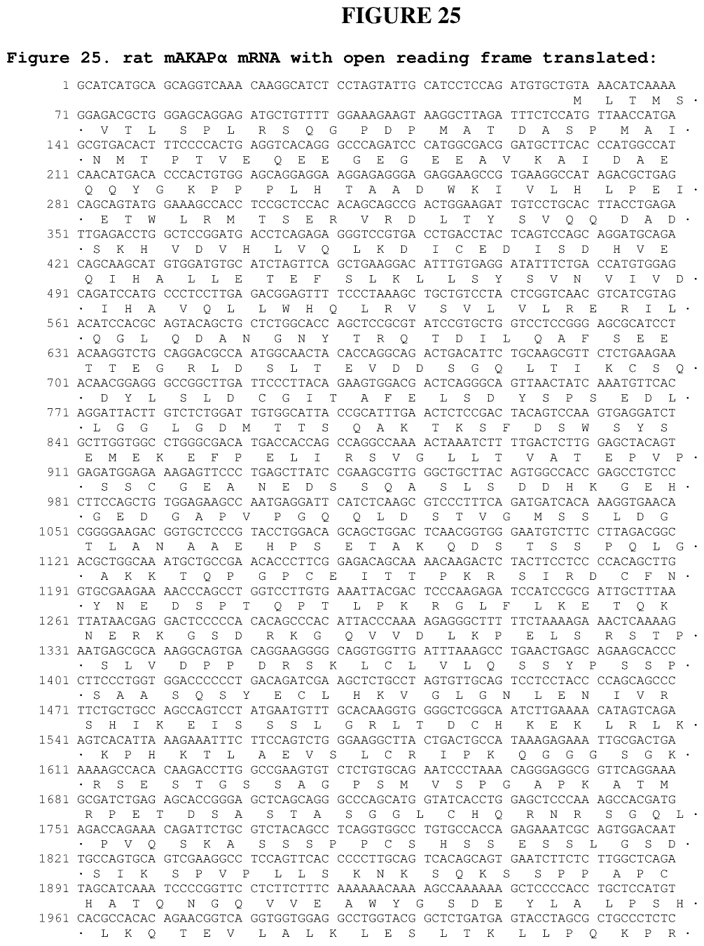

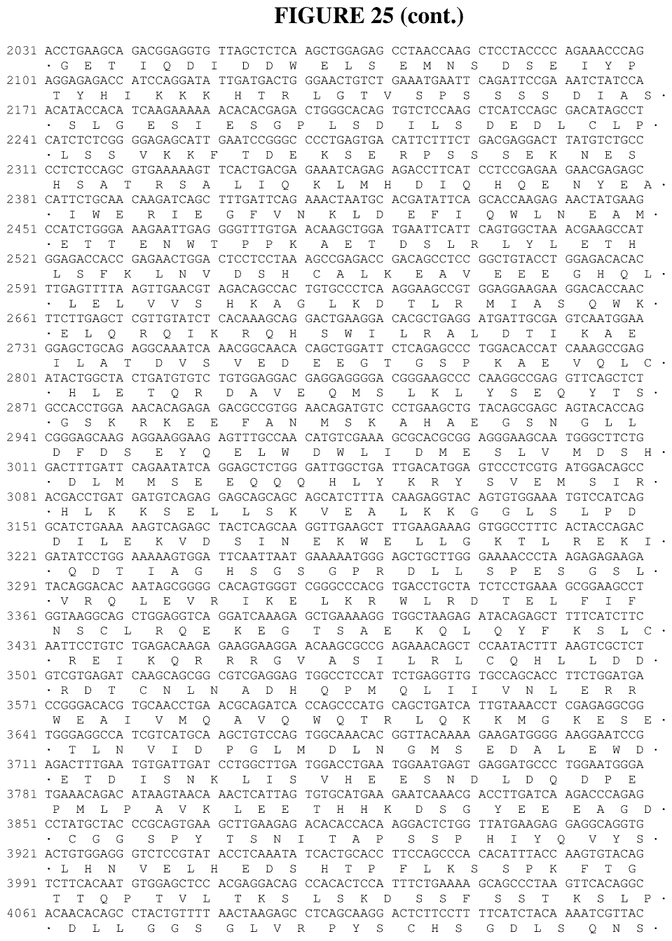

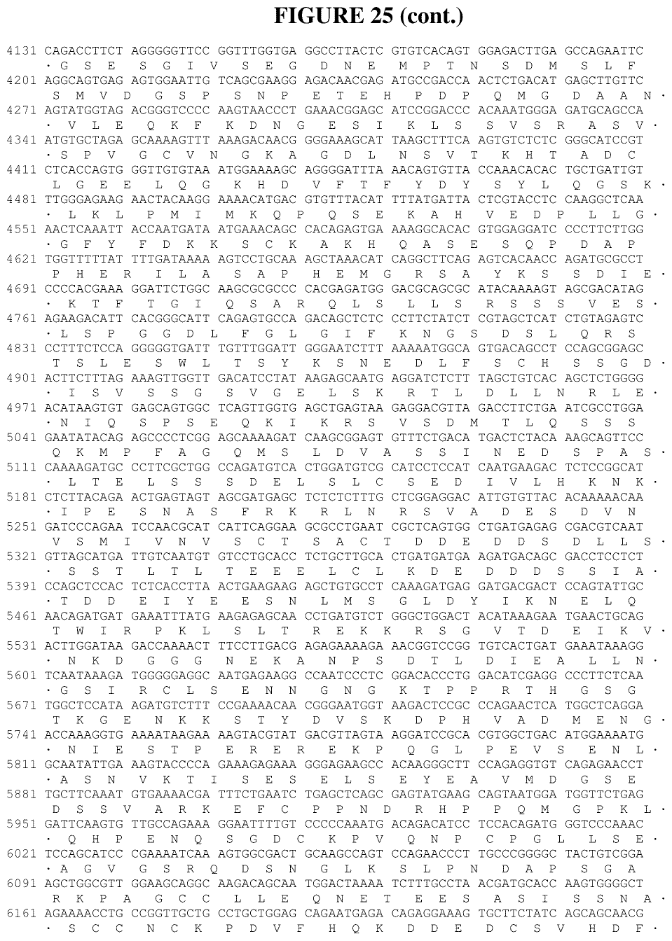

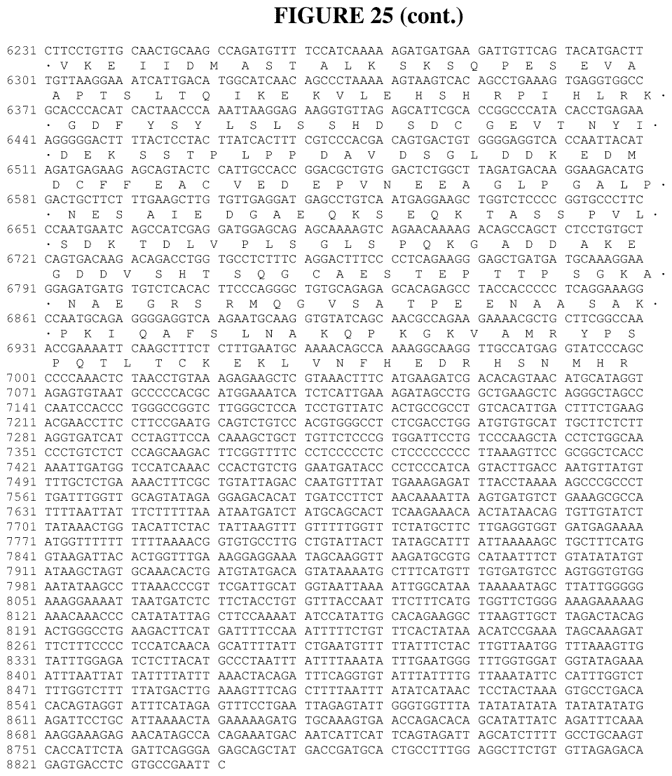

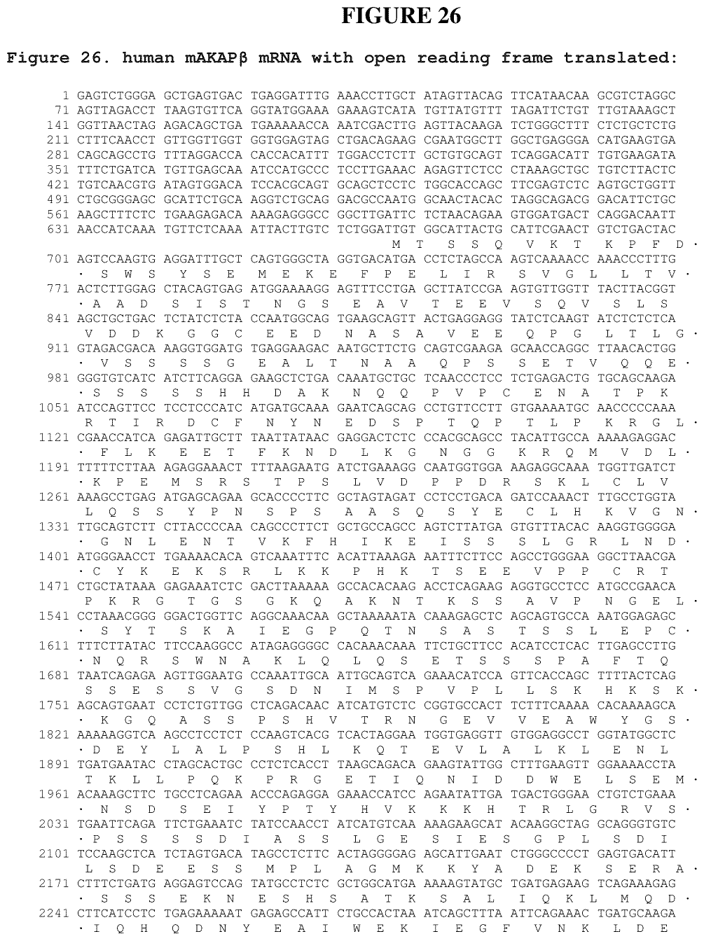

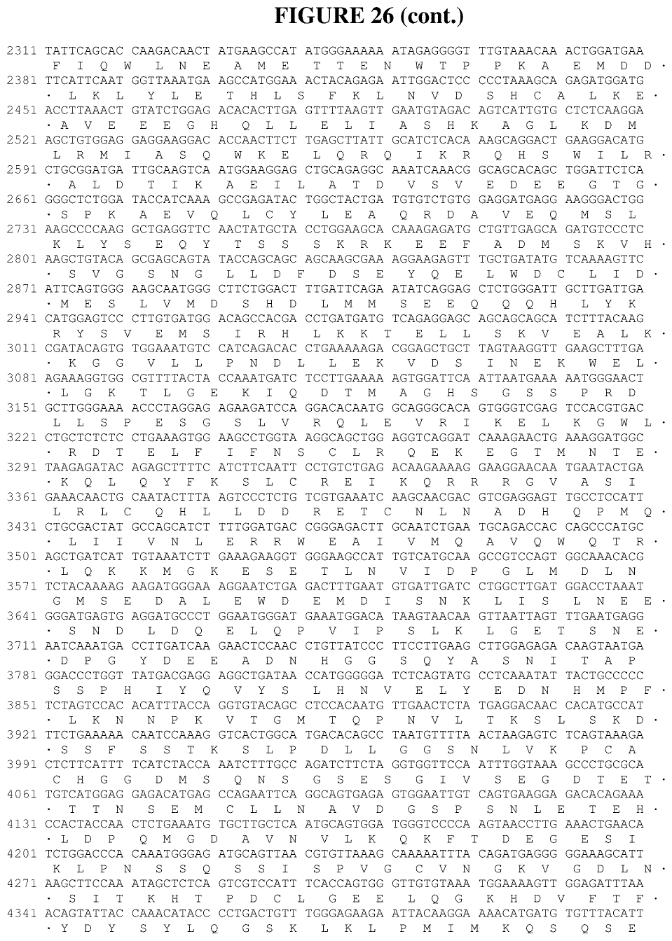

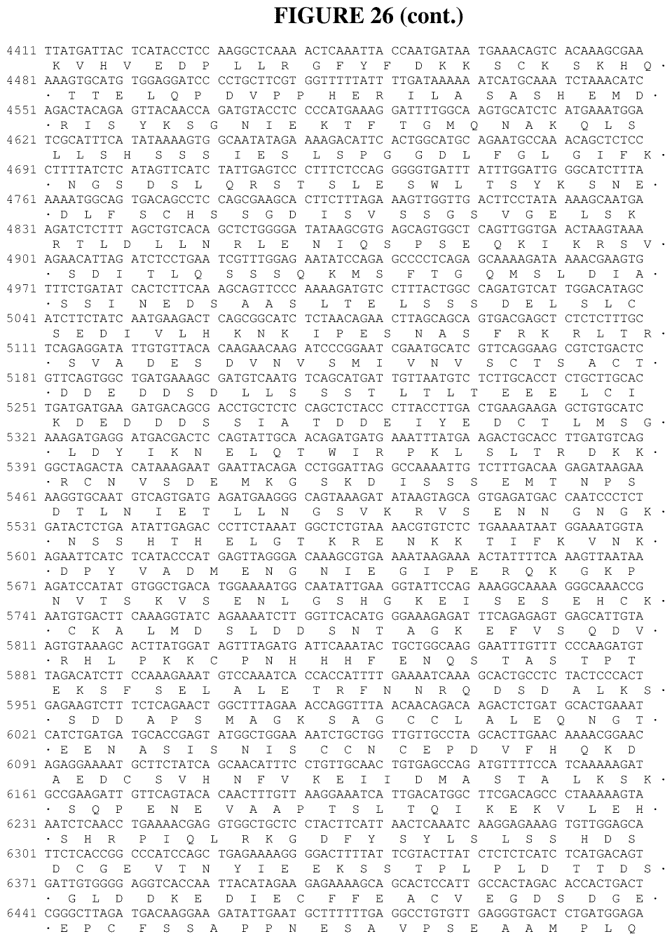

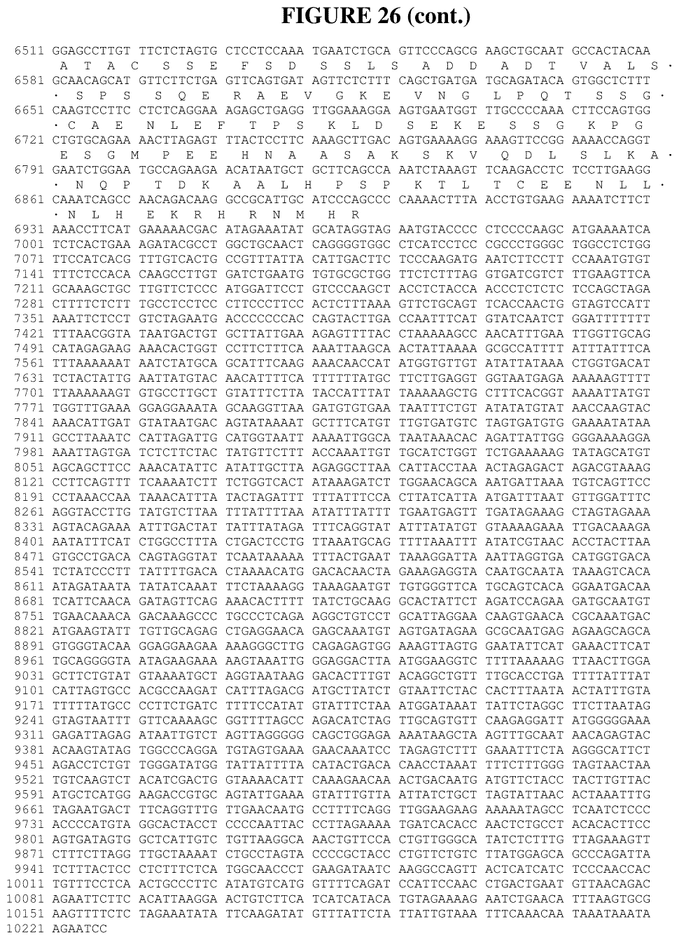

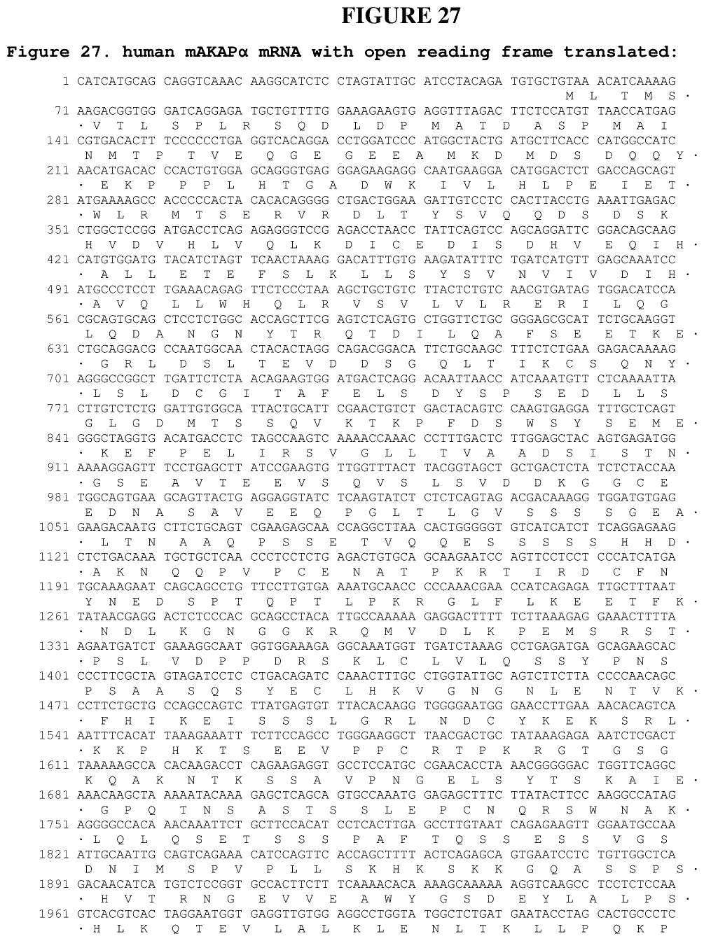

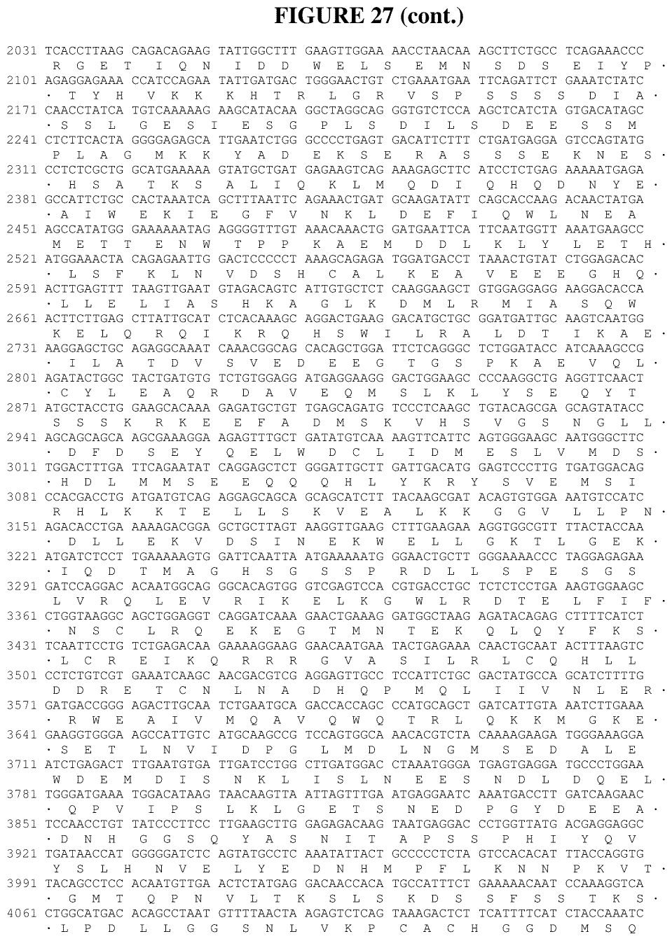

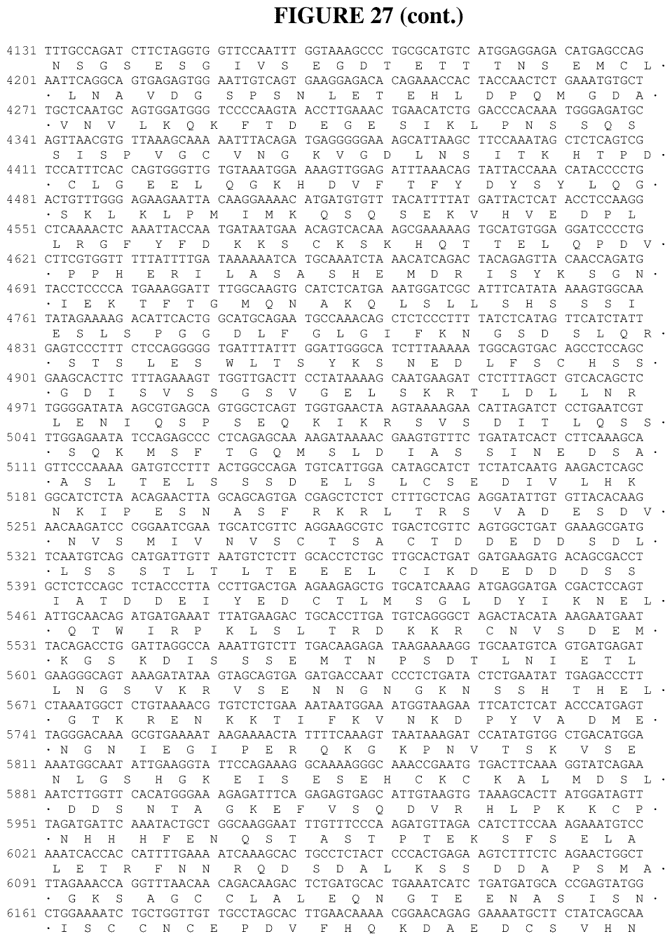

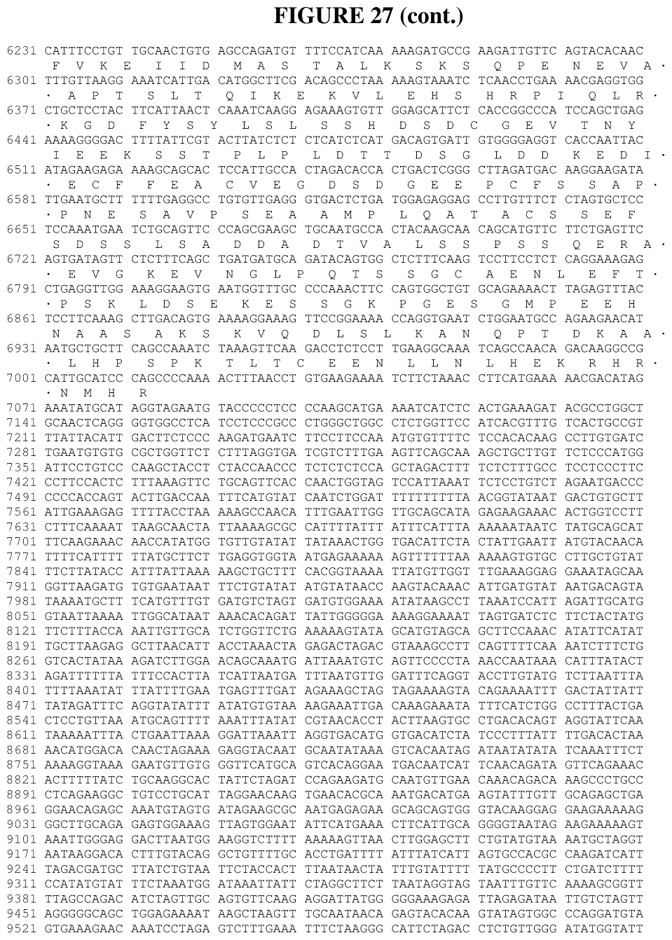

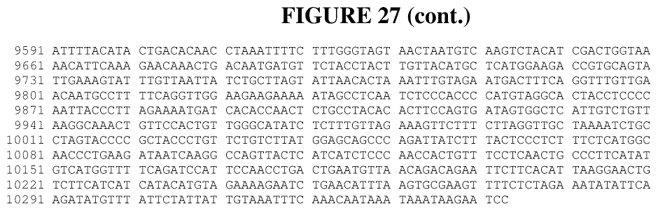

The present inventors now disclose a new mechanism of action for mAKAPβ-bound PP2A in the cardiac myocyte and the therapeutic implications of this mechanism. The inventors show that the transcription factor SRF is phosphorylated at Ser¹⁰³ by mAKAPβ-bound RSK3 (FIG. 19) and that SRF phosphorylation at Ser¹⁰³ constitutes an epigenetic switch promoting concentric cardiac myocyte hypertrophy (FIG. 20). Importantly, it is disclosed that SRF Ser¹⁰³ can be dephosphorylated by PP2A bound to the mAKAPβ scaffold (FIGS. 19 and 21). SRF Ser¹⁰³ phosphorylation is shown to induce concentric myocyte hypertrophy (FIG. 20). These findings constitute the discovery of a novel mechanism for the regulation of cardiac myocyte morphology and an unexpected function for mAKAPβ-bound PP2A. In particular, the inventors disclose that consistent with the role of PP2A as a phosphatase for mAKAPβ-bound SRF, displacement of PP2A from mAKAPβ in vitro will promote SRF Ser¹⁰³ phosphorylation in cardiac mycoytes (FIG. 19) and concentric cardiac myocyte hypertrophy (FIG. 20) and in vivo will provide protection against the development of systolic dysfunction after myocardial infarction in mice (FIG. 23).

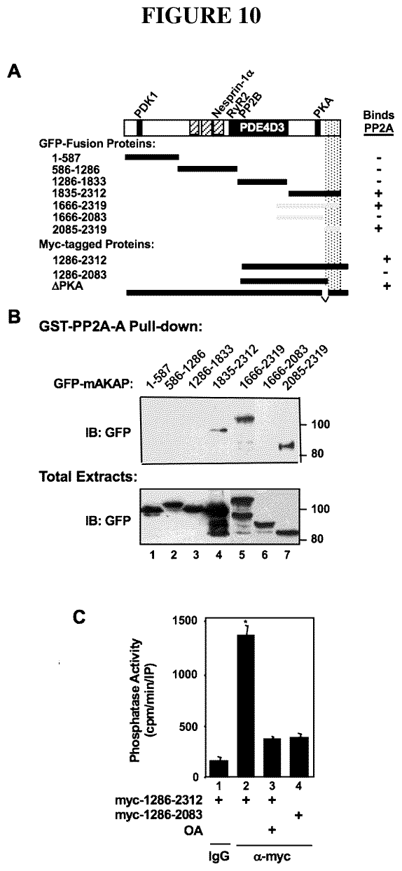

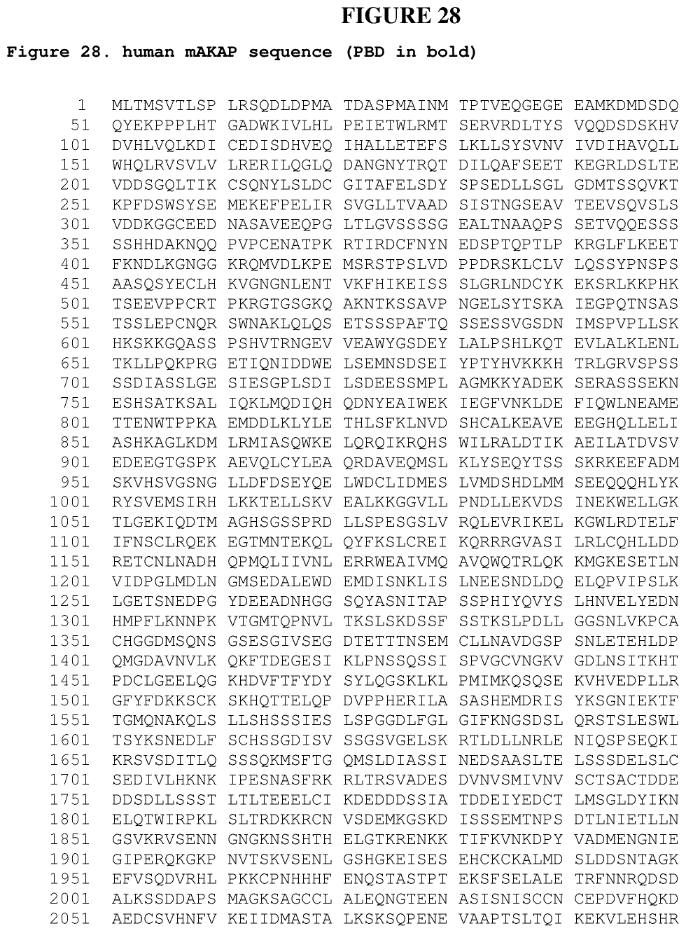

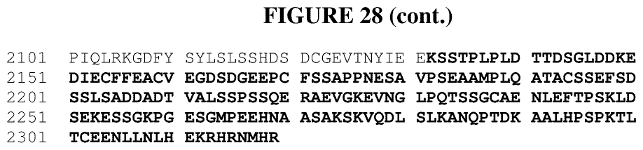

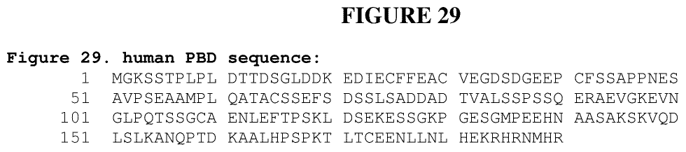

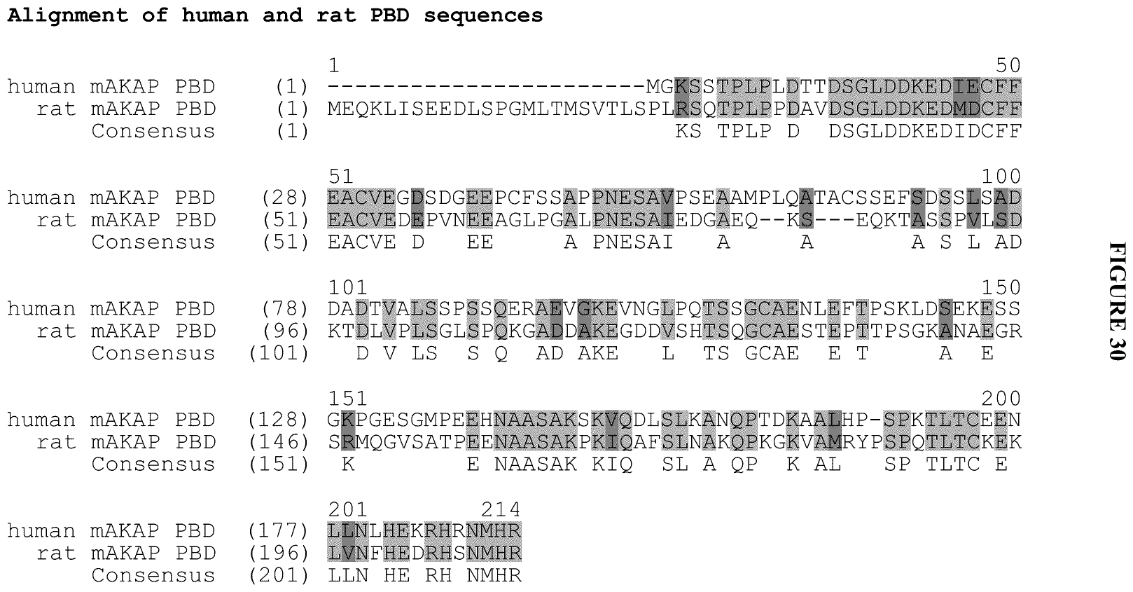

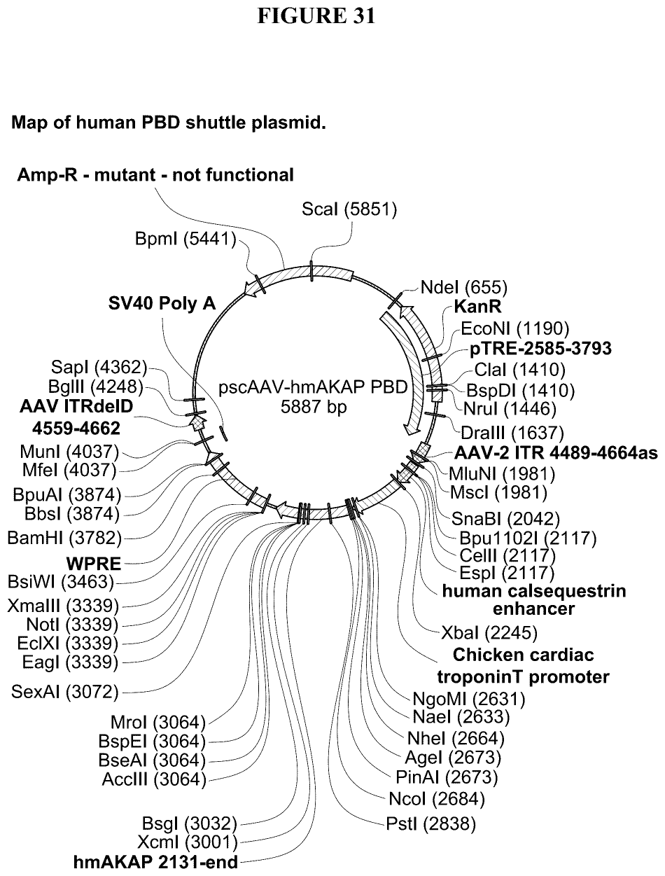

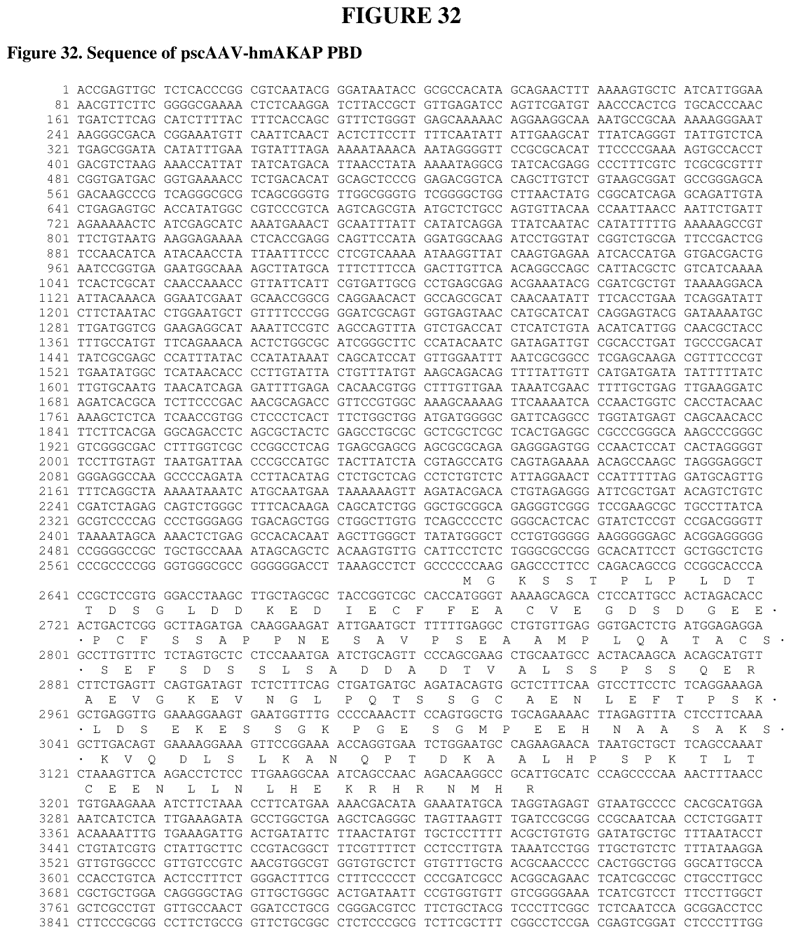

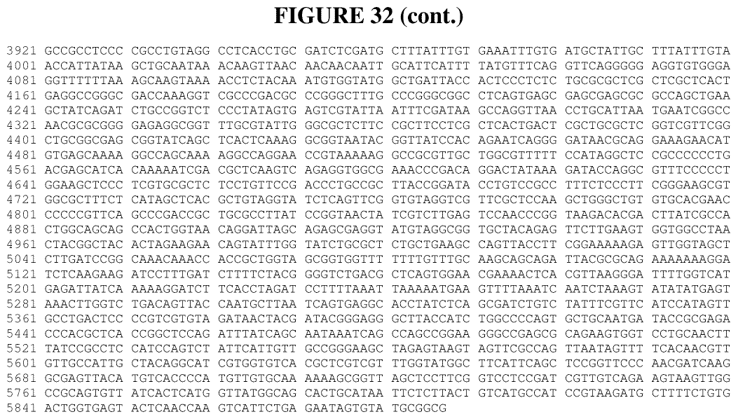

Inhibition of PP2A binding to mAKAPβ can be achieved by expression of a competing peptide comprising rat mAKAPβ 2134-2314 (FIG. 19) or 2132-2319 of human mAKAPβ, representing a new refinement in the mapping of the PP2A binding site on mAKAPβ and the first demonstration for heart disease in vivo of the inhibition of mAKAP-PP2A binding. Note that the C-terminal domain of human mAKAP homologous to that in rat mAKAP was also shown to bind PP2A (FIG. 10). Therefore the human sequence (human mAKAP amino acid residues homologous 2132-2319) to rat mAKAP 2134-2314 shown in FIGS. 28-30 is also expected to bind PP2A and constitute a PP2A-mAKAP binding competing peptide.

Effective delivery of PP2A anchoring disruptor peptides via viral-based gene therapy vectors are demonstrated by efficacy in the mouse infarction model (FIG. 23). Alternatively, delivery of such peptides that might inhibit PP2A-mAKAPβ interaction can be enhanced by the use of cell-penetrating sequences such as the transactivator of transcription peptide and polyarginine tails, or conjugation with lipid-derived groups such as stearate. Stability may also be enhanced by the use of peptidomimetics [i.e., peptides with structural modifications in the original sequence giving protection against exo- and endoproteases without affecting the structural and functional properties of the peptide.]

The inventors have also found that small molecule disruptors can be used to target specific interaction within AKAP-based complexes. Small molecule disruptors can be identified by combining rational design and screening approaches. Such compounds can be designed to target-specific binding surfaces on AKAPs, to disrupt the interaction between AKAPs and PP2A in cardiomyocytes and to enhance the contractility of intact hearts for the treatment of chronic heart failure.

The present invention relates to methods of treating any cardiac condition which is initiated through the interaction of PP2A and mAKAPβ. Such cardiac dysfunction can result in signs and symptoms such as shortness of breath and fatigue, and can have various causes, including, but not limited to hypertension, coronary artery disease, myocardial infarction, valvular disease, primary cardiomyopathy, congenital heart disease, arrhythmia, pulmonary disease, diabetes, anemia, hyperthyroidism and other systemic diseases.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (4th Ed., 2012); “Current Protocols in Molecular Biology” Volumes I-III [Ausubel, R. M., ed. (1994)]; “Cell Biology: A Laboratory Handbook” Volumes I-III [J. E. Celis, 3rd ed. (2005))]; “Current Protocols in Immunology” Volumes I-III [Coligan, J. E., ed. (2005)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984); C. Machida, “Viral Vectors for Gene Therapy: Methods and Protocols” (2010); J. Reidhaar-Olson and C. Rondinone, “Therapeutic Applications of RNAi: Methods and Protocols” (2009).

The following definitions and acronyms are used herein:
AC5—adenylyl cyclase type 5
ACE—angiotensin-converting enzyme
ANF atrial natriuretic factor
ARVM—adult rat ventricular myocyte
CaN—calcineurin
CArG box—CC(A/T)₆GG
CPT-cAMP—8-(4-chlorophenylthio)adenosine 3′,5′-cyclic monophosphate
CsA—cyclosporin A
CTKD—C-terminal kinase domain
ERK—extracellular signal-regulated kinase
FBS—fetal bovine serum
Fsk—forskolin
GFP—green fluorescent protein
GPCR—G-protein coupled receptor; HDAC—histone deacetylase
Gs—stimulatory G protein
GST—glutathione-S-transferase; HIF1α—hypoxia-inducible factor 1α
HFrEF—heart failure with reduced ejection fraction
IBMX—3-isolbutyl-1-methylxanthine
Iso—isoproterenol
LIF—leukemia inhibitory factor
MADS—(MCM1, agamous, deficiens, SRF) domain—mediates DNA binding to CArG box CC(A/T)₆GG serum response elements (SRE); the MADS-box gene family got its name later as an acronym referring to the four founding members, ignoring ARG80:
   MCM1 from the budding yeast, Saccharomyces cerevisiae,
   AGAMOUS from the thale cress Arabidopsis thaliana,
   DEFICIENS from the snapdragon Antirrhinum majus,[10]
   SRF from the human Homo sapiens.
mAKAP—muscle A—kinase anchoring protein
mAKAPα—alternatively spliced isoform expressed in neurons; 255 kDa
mAKAPβ—alternatively spliced isoform expressed in striated myocytes; 230 kDa
MAPK—mitogen-activated protein kinase
MEF2—myocyte enhancer factor-2
MgAc—magnesium acetate
MI—myocardial infarction
NCX1—sodium/calcium exchanger
NFATc—nuclear factor of activate T-cell
NRVM—neonatal rat ventricular myocyte
NTKD—N-terminal kinase domain
OA—Okadaic acid
PBD—“PP2A binding domain” of mAKAP that binds PP2A and that when expressed attenuates eccentric hypertrophy
PDE4D3—cAMP-specific phosphodiesterase type 4D3
PDK1—3′phosphoinositide-dependent kinase 1
PE—phenylephrine
PHD—prolyl hydroxylase
PI4P—phosphatidylinositol-4-phosphate
PKA—protein kinase A
PKD—protein kinase D
PKI—protein kinase inhibitor
PLCε—phospholipase Cε
PKA—cAMP-dependent protein kinase
PP2A—protein (serine-threonine) phosphatase—dephosphorylates SRF Ser¹⁰³
PP2B—calcium/calmodulin-dependent protein phosphatase 2B
RBD—isoform-specific N-terminal RSK3 domain binds a discrete “RSK3-binding domain” within mAKAPβ at residues 1694-1833 (RBD)
RSK—p90 ribosomal S6 kinase
RyR2—type 2 ryanodine receptor
siRNA—small interfering RNA oligonucleotide
shRNA—short hairpin RNA
SRE—serum response elements
SRF—serum response factor—transcription factor (SRF Ser¹⁰³ phosphorylation induces concentric myocyte and cardiac hypertrophy)
siRNA—small interfering RNA
TAC transverse aortic constriction
TCA—trichloroacetic acid
VSV—vesicular stomatitis virus

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well known and commonly used in the art. Certain experimental techniques, not specifically defined, are generally performed according to conventional methods well known in the art and as described in various general and more specific references that are cited and discussed throughout the present specification. For purposes of the clarity, following terms are defined below.

The present invention recognizes that the interaction of PP2A and mAKAPβ mediates various intracellular signals and pathways which lead to cardiac myocyte hypertrophy and/or dysfunction. As such, the present inventors have discovered various methods of inhibiting that interaction in order to prevent and/or treat cardiac myocyte hypertrophy and/or dysfunction.

Thus, the present invention includes a method for protecting the heart from damage, by administering to a patient at risk of such damage, a pharmaceutically effective amount of a composition, which inhibits the interaction of PP2A and mAKAPβ. It should be appreciated that “a pharmaceutically effective amount” can be empirically determined based upon the method of delivery, and will vary according to the method of delivery.

The invention also relates to a method of treating heart disease, by administering to a patient a pharmaceutically effective amount of a composition, which inhibits the interaction of PP2A and mAKAPβ.

The invention also relates to compositions which inhibit the interaction of PP2A and mAKAPβ. In particular embodiments, these inhibiting compositions or “inhibitors” include peptide inhibitors, which can be administered by any known method, including by gene therapy delivery. In other embodiments, the inhibitors can be small molecule inhibitors.

Specifically, the present invention is directed to methods and compositions for treating or protecting the heart from damage, by administering to a patient at risk of such damage, a pharmaceutically effective amount of a composition which (1) inhibits the interaction of PP2A and mAKAPβ; (2) inhibits the activity of PP2A and mAKAPβ; or (3) inhibits the expression of PP2A and mAKAPβ.

The invention also relates to methods of treating or protecting the heart from damage, by administering to a patient at risk of such damage, a pharmaceutically effective amount of a composition which inhibits a cellular process mediated by the anchoring of PP2A.

In one embodiment, the composition includes an mAKAPβ peptide. In a preferred embodiment, the mAKAPβ peptide is obtained from the carboxy terminus of the mAKAPβ amino acid sequence. In a particularly preferred embodiment, the mAKAPβ peptide is at least a fragment of amino acids 2083-2319 of the mAKAPβ amino acid sequence.

In one preferred embodiment, the mAKAPβ peptide is at least a fragment of amino acids 2132-2319 of the mAKAPβ amino acid sequence.

In another embodiment, the composition includes a small interfering RNA siRNA that inhibits the expression of either or both of PP2A and mAKAPβ. In a preferred embodiment, the siRNA that inhibits the expression of mAKAPβ is generated in vivo following administration of a short hairpin RNA expression vector or biologic agent (shRNA).

The composition of the invention can be administered directly or can be administered using a viral vector. In a preferred embodiment, the vector is adeno-associated virus (AAV).

In another embodiment, the composition includes a small molecule inhibitor. In preferred embodiments, the small molecule is a PP2A inhibitor.

In another embodiment, the composition includes a molecule that inhibits the binding, expression or activity of mAKAPβ. In a preferred embodiment, the molecule is a mAKAPβ peptide. The molecule may be expressed using a viral vector, including adeno-associated virus (AAV).

In yet another embodiment, the composition includes a molecule that interferes with mAKAPβ-mediated cellular processes. In preferred embodiments, the molecule interferes with the anchoring of PP2A.

The invention also relates to diagnostic assays for determining a propensity for heart disease, wherein the binding interaction of PP2A and mAKAPβ is measured, either directly, or by measuring a downstream effect of the binding of PP2A and mAKAPβ. The invention also provides a test kit for such an assay.

In still other embodiments, the inhibitors include any molecule that inhibits the expression of PP2A and mAKAPβ, including antisense RNA, ribozymes and small interfering RNA (siRNA), including shRNA.

The invention also includes an assay system for screening of potential drugs effective to inhibit the expression and/or binding of PP2A and mAKAPβ. In one instance, the test drug could be administered to a cellular sample with the PP2A and mAKAPβ, or an extract containing the PP2A and mAKAPβ, to determine its effect upon the binding activity of the PP2A and mAKAPβ, by comparison with a control. The invention also provides a test kit for such an assay.

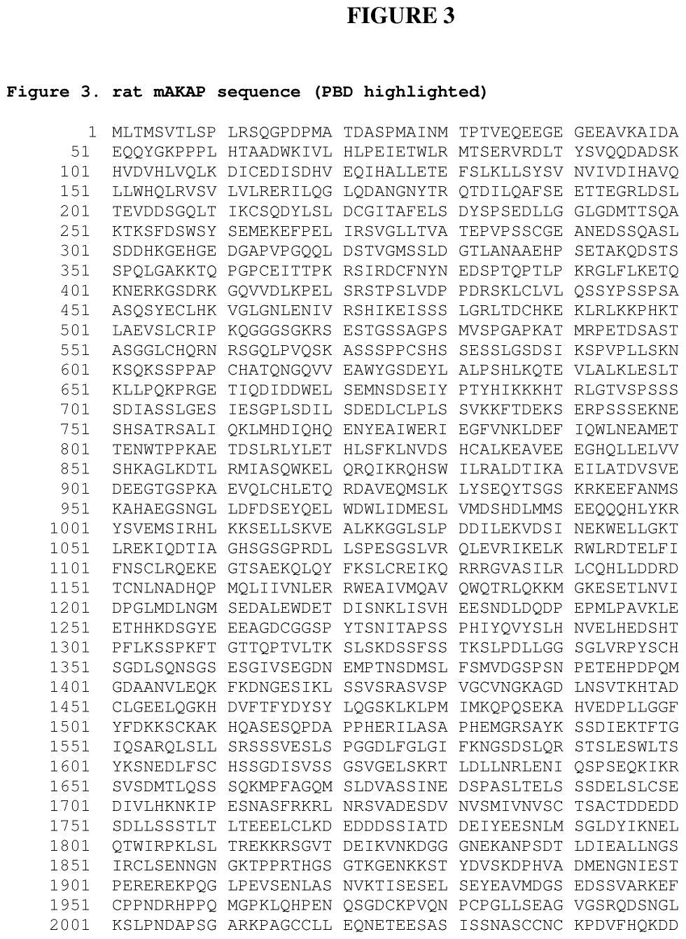

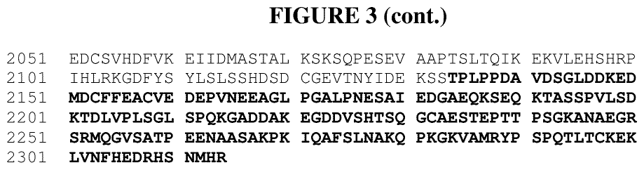

In preparing the peptide compositions of the invention, all or part of the PP2A or mAKAP (FIG. 3 or FIG. 28) amino acid sequence may be used. In one embodiment, the carboxy-terminal region of the mAKAPβ protein is used as an inhibitor. Preferably, at least 10 amino acids of the mAKAP sequence are used. More preferably, at least 25 amino acids of the mAKAP sequence are used. Most preferably, peptide segments from amino acids 2132-2319 of mAKAP are used.

It should be appreciated that various amino acid substitutions, deletions or insertions may also enhance the ability of the inhibiting peptide to inhibit the interaction of PP2A and mAKAPβ. A substitution mutation of this sort can be made to change an amino acid in the resulting protein in a non-conservative manner (i.e., by changing an amino acid belonging to a grouping of amino acids having a particular size or characteristic to an amino acid belonging to another grouping) or in a conservative manner (i.e., by changing an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes, which do not significantly alter the activity, or binding characteristics of the resulting protein.

The following is one example of various groupings of amino acids:

Amino acids with nonpolar R groups: Alanine, Valine, Leucine, Isoleucine, Proline, Phenylalanine, Tryptophan, Methionine.

Amino acids with uncharged polar R groups: Glycine, Serine, Threonine, Cysteine, Tyrosine, Asparagine, Glutamine.

Amino acids with charged polar R groups (negatively charged at pH 6.0): Aspartic acid, Glutamic acid.

Basic amino acids (positively charged at pH 6.0): Lysine, Arginine, Histidine (at pH 6.0).

Another grouping may be those amino acids with phenyl groups: Phenylalanine, Tryptophan, Tyrosine.

Another grouping may be according to molecular weight (i.e., size of R groups): Glycine (75), Alanine (89), Serine (105), Proline (115), Valine (117), Threonine (119), Cysteine (121), Leucine (131), Isoleucine (131), Asparagine (132), Aspartic acid (133), Glutamine (146), Lysine (146), Glutamic acid (147), Methionine (149), Histidine (at pH 6.0) (155), Phenylalanine (165), Arginine (174), Tyrosine (181), Tryptophan (204).

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces ß-turns in the protein's structure. Two amino acid sequences are “substantially homologous” when at least about 70% of the amino acid residues (preferably at least about 80%, and most preferably at least about 90 or 95%) are identical, or represent conservative substitutions.

Likewise, nucleotide sequences utilized in accordance with the invention can also be subjected to substitution, deletion or insertion. Where codons encoding a particular amino acid are degenerate, any codon which codes for a particular amino acid may be used. In addition, where it is desired to substitute one amino acid for another, one can modify the nucleotide sequence according to the known genetic code.

Nucleotides and oligonucleotides may also be modified. U.S. Pat. No. 7,807,816, which is incorporated by reference in its entirety, and particularly for its description of modified nucleotides and oligonucleotides, describes exemplary modifications.

Two nucleotide sequences are “substantially homologous” or “substantially identical” when at least about 70% of the nucleotides (preferably at least about 80%, and most preferably at least about 90 or 95%) are identical.

Two nucleotide sequences are “substantially complementary” when at least about 70% of the nucleotides (preferably at least about 80%, and most preferably at least about 90 or 95%) are able to hydrogen bond to a target sequence.

The term “standard hybridization conditions” refers to salt and temperature conditions substantially equivalent to 5×SSC and 65 C for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20 C below the predicted or determined Tₘ with washes of higher stringency, if desired.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to prevent, and preferably reduce by at least about 30 percent, more preferably by at least 50 percent, most preferably by at least 90 percent, a clinically significant change in a cardiac myocyte feature.

The preparation of therapeutic compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment, as well as a small molecule inhibitor, can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic compositions of the invention are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition of PP2A-mAKAPβ binding desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

Because of the necessity for the inhibitor to reach the cytosol, a peptide in accordance with the invention may need to be modified in order to allow its transfer across cell membranes, or may need to be expressed by a vector which encodes the peptide inhibitor. Likewise, a nucleic acid inhibitor (including siRNAs, shRNAs and antisense RNAs) can be expressed by a vector. Any vector capable of entering the cells to be targeted may be used in accordance with the invention. In particular, viral vectors are able to “infect” the cell and express the desired RNA or peptide. Any viral vector capable of “infecting” the cell may be used. A particularly preferred viral vector is adeno-associated virus (AAV).

siRNAs inhibit translation of target mRNAs via a process called RNA interference. When the siRNA is perfectly complementary to the target mRNA, siRNA act by promoting mRNA degradation. shRNAs, as a specialized type of siRNA, have certain advantages over siRNAs that are produced as oligonucleotides. siRNA oligonucleotides are typically synthesized in the laboratory and are delivered to the cell using delivery systems that deliver the siRNA to the cytoplasm. In contrast, shRNAs are expressed as minigenes delivered via vectors to the cell nucleus, where following transcription, the shRNA are processed by cellular enzymes such as Drosha and Dicer into mature siRNA species. siRNAs are usually 99% degraded after 48 hours, while shRNAs can be expressed up to 3 years. Morover, shRNAs can be delivered in much lower copy number than siRNA (5 copies vs. low nM), and are much less likely to produce off-target effects, immune activation, inflammation and toxicity. While siRNAs are suitable for acute disease conditions where high doses are tolerable, shRNAs are suitable for chronic, life threatening diseases or disorders where low doses are desired. (http://www.benitec.com/technology/sirna-vs-shrna)

Guidelines for the design of siRNAs and shRNAs can be found in Elbashir (2001) and at various websites including https://www.thermofisher.com/us/en/home/references/ambion-tech-support/rnai-sirna/general-articles/-sirna-design-guidelines.html and http://www.invivogen.com/review-sirna-shrna-design, all of which are hereby incorporated by reference in their entireties. Preferably, the first nucleotide is an A or a G. siRNAs of 25-29 nucleotides may be more effective than shorter ones, but shRNAs with duplex length 19-21 seem to be as effective as longer ones. siRNAs and shRNAs are preferably 19-29 nucleotides. Loop sequences in shRNAs may be 3-9 nucleotides in length, with 5, 7 or 9 nucleotides preferred.

With respect to small molecule inhibitors, any small molecule that inhibits the interaction of PP2A and mAKAPβ may be used. In addition, any small molecules that inhibit the activity of PP2A and/or mAKAPβ may be used.

Small molecules with similar structures and functionalities can likewise be determined by rational and screening approaches.

Likewise, any small molecules that inhibit the expression of PP2A and/or mAKAPβ may be used.

In yet more detail, the present invention is described by the following items which represent preferred embodiments thereof:
1. A method of treating or preventing heart failure with reduced ejection fraction, comprising administering to cardiac cells of a patient a composition that maintains a level of phosphorylation on serum response factor (SRF).
2. The method of Item 1, wherein SRF is phosphorylated on Ser¹⁰³.
3. The method of Item 1, wherein dephosphorylation activity of protein (serine-threonine) phosphatase 2A (PP2A) is inhibited.
4. The method of Item 3, wherein anchoring of PP2A to muscle A-kinase anchoring protein (mAKAPβ) is inhibited.
5. The method of Item 4, wherein the composition comprises a fragment of mAKAPβ.
6. The method of Item 5, wherein the composition comprises an amino acid sequence having at least 90% sequence identity to a fragment of mAKAPβ.
7. The method of Item 5, wherein the composition comprises a fragment of amino acids 2132-2319 of mAKAP.
8. The method of Item 5, wherein the composition comprises amino acids 2132-2319 of mAKAP.
9. The method of Item 4, wherein the composition comprises a fragment of PP2A.
10. The method of Item 4, wherein said composition comprises a vector that encodes a fragment of mAKAP.
11. The method of Item 4, wherein said composition comprises a vector that encodes an amino acid sequence having at least 90% sequence identity to a fragment of mAKAP.
12. The method of Item 10, wherein the vector encodes a fragment of amino acids 2132-2319 of mAKAP.
13. The method of Item 10, wherein the vector encodes amino acids 2132-2319 of mAKAP.
14. The method of Item 10, wherein the vector is adeno-associated virus (AAV).
15. A composition that encodes a molecule that inhibits the anchoring of PP2A to mAKAP.
16. The composition of Item 15, wherein the molecule comprises a fragment of mAKAP.
17. The composition of Item 15, comprising an amino acid sequence having at least 90% sequence identity to a fragment of mAKAP.
18. The composition of Item 16, comprising a fragment of amino acids 2132-2319 of mAKAP.
19. The composition of Item 16, comprising amino acids 2132-2319 of mAKAPβ.
20. The composition of Item 15, comprising a fragment of PP2A.
21. A composition comprising a vector that encodes a molecule that inhibits the anchoring of PP2A to mAKAP.
22. The composition of Item 21, wherein the vector encodes a fragment of mAKAP.
23. The composition of Item 21, wherein the vector encodes an amino acid sequence having at least 90% sequence identity to a fragment of mAKAP.
24. The composition of Item 21, wherein the vector encodes a fragment of amino acids 2132-2319 of mAKAP.
25. The composition of Item 21, wherein the vector encodes amino acids 2132-2319 of mAKAP.
26. The composition of Item 21, wherein the vector encodes a fragment of PP2A.
27. The composition of Item 21, wherein the vector is adeno-associated virus (AAV).

The following examples are provided to aid the understanding of the present invention, the true scope of which is set forth in the appended claims. It is understood that modifications can be made in the procedures set forth without departing from the spirit of the invention.

### EXAMPLES

The compositions and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the processes, formulations and/or methods of the invention may be made without departing from the spirit of the invention and the scope of the appended claims.

### Example 1

SRF Regulation by mAKAPβ Signalosomes

### Materials and Methods

Neonatal Rat Ventricular Myocyte Culture: 1-3 day old Sprague-Dawley rats were decapitated, and the excised hearts placed in 1×ADS Buffer (116 mM NaCl, 20 mM HEPES, 1 mM NaH₂PO₄, 5.5 mM glucose, 5.4 mM KCl, 0.8 mM MgSO₄, pH 7.35). The atria were carefully removed and the blood washed away. The ventricles were minced and incubated with 15 mL 1×ADS Buffer containing 3.3 mg type II collagenase (Worthington, 230 U/mg) and 9 mg Pancreatin (Sigma) at 37° C. with gentle shaking. After 15 minutes, the dissociated cardiac myocytes were separated by centrifugation at 50 g for 1 minute, resuspended in 4 mL horse serum and incubated at 37° C. with occasional agitation. The steps for enzymatic digestion and isolation of myocytes were repeated 10-12 times to maximize yield. The myocytes were pooled and spun down again at 50 g for 2 minutes and resuspended in Maintenance Medium (DMEM:M199, 4:1) supplemented with 10% horse serum and 5% fetal bovine serum. To remove any contaminating fibroblasts, the cells were pre-plated for 1 hour before plating on gelatin-coated tissue culture plastic ware. This procedure yields >90% pure cardiac myocytes. After 1 day culture, the media was changed to maintenance medium containing 0.1 mM bromodeoxyuridine to suppress fibroblast growth.

Adult rat ventricular myocyte isolation and culture: 2-3 month old rats were anesthetized using Ketamine (80-100 mg/kg) and Xylazine (5-10 mg/kg) IP following 1000 U heparinization for cardiac excision. The heart was transferred immediately into chilled perfusion buffer (NaCl 120 mM, KCl 5.4 mM, Na₂HPO4·7H₂O 1.2 mM, NaHCO₃ 20.0 mM, MgCl₂·6H₂O 1.6 mM, Taurine 5 mM, Glucose 5.6 mM, 2,3-Butanedione monoxime 10 mM) pre-equilibrated with 95% O₂ and 5% CO₂. After removal of extraneous tissue, the heart was attached via the aorta to a Harvard Langendorff apparatus cannula. Ca²⁺-free perfusion was used to flush out remaining blood with a constant rate of 8-10 mL/min at 37° C. The heart was then digested through circulatory perfusion with 50 mL perfusion buffer containing 125 mg type II collagenase (Worthington, 245 U/mg), 0.1 mg protease (Sigma type XIV) and 0.1% BSA. After perfusion, the atria were removed and the ventricular myocytes dissociated by slicing and repetitive pipetting. The debris was filtered by a 200 μm nylon mesh, and the myocytes collected by one minute centrifugation at 50 g. Ca²⁺ concentration in the buffer was gradually recovered to 1.8 mM and the myocytes were resuspended in ACCT medium (M199 Medium (Invitrogen 11150-059), Creatine 5 mM, L-carnitine 2 mM, Taurine 5 mM, HEPES 25 mM, 2,3-Butanedione monoxime 10 mM, BSA 0.2% and 1× Insulin-Transferrin-Selenium Supplement) and plated on 10 μg/ml laminin pre-coated dishes. Cells were washed with ACCT medium 1.5 hours after plating and subjected to adenoviral infection or siRNA transfection, in which 100-200 Multiplicity of Infection (MOI) of adenovirus and 100 nmol/L siRNA mixed with Dharmafect1 (Dharmacon) were used, respectively. Adrenergic agonists were added the next day, with biochemical assay and morphological measurement performed after 24 hours of stimulation.

Other Cell Culture: HEK293 and COS-7 cells were maintained in DMEM with 10% FBS and 1% P/S. These cells were transiently transfected with Lipofectamine 2000 (Invitrogen) or infected with adenovirus and Adeno-X Tet-Off virus (Clontech) as suggested by the manufacturers.

Luciferase Assays: 225,000 neonatal rat ventricular myocytes in 24 well dishes were transfected with control or RSK3 specific siRNA oligonucleotides (10 nM) and Dharmafect1 reagent (Thermofisher). The following day, following washing the cells with media, the myocytes were re-transfected with 100 ng SRE-luc (firefly luciferase) and 100 ng −36Prl-rluc (renilla luciferase) reporter plasmids and Transfast reagent for one hour and then cultured in media with 4% horse serum overnight, before washing with media and incubating for one day in the absence or presence of 10 μM PE. Samples were collected in 100 μl PLB and assayed using the Promega Dual Luciferase Kit and a Berthold Centro X luminometer.

Co-Immunoprecipitation: Tissues were homogenized using a Polytron or cells were lysed in IP buffer (50 mM HEPES pH 7.4, 150 mM NaCl, 5 mM EDTA, 10% glycerol, 1% Triton-X 100, 1 mM DTT) with an inhibitor cocktail (1 μg/ml leupeptin, 1 μg/ml pepstatin, 1 mM benzamidine, 1 mM AEBSF, 50 mM NaF, 1 mM sodium orthovanadate). Soluble proteins were separated by centrifugation at 3-10,000 g for 10 minutes. Antibodies and protein-G agarose beads (50% slurry, Upstate) were added to extracts and incubated overnight with rocking at 4° C. Beads were washed four times at 4° C. with IP buffer. Bound proteins were size-fractionated on SDS-PAGE gels and developed by immunoblotting as previously described using a Fujifilm LAS-3000 or GE-A1600 imaging system (46). Protein markers were Precision Plus Protein Standards (Bio-Rad, 1610373).

Immunocytochemistry: Myocytes on coverslips were fixed in 3.7% formaldehyde in PBS for 1 hour, permeabilized with 0.3% Triton X-100, and blocked in PBS containing 0.2% BSA and 1% horse serum. The slides were then sequentially incubated for 1 hour with primary and Alexa fluorescent dye-conjugated specific-secondary antibodies (Invitrogen, 1:1000) diluted in blocking buffer. The slips were washed three times with blocking buffer. 1 μg/mL Hoechst 33258 was included in the last wash stop to label nuclei. Slides were sealed in SlowFade Gold antifade buffer (Invitrogen, S36938) for fluorescent microscopy. Wide-field images were acquired using a Leica DM4000 Microscope.

GST-SRF phosphorylation assays: GST-SRF protein was purified using BL21 E. coli and glutathione-sepharose as previously described (Vargas et al. 2012). GST-SRF on beads was incubated with 0.5 μg active recombinant full-length His₆-tagged human RSK3 (Millipore 14-462)+/−50 nM BI-D1870 in ATP-containing kinase buffer for 30 minutes. The GST-SRF beads were then either eluted with Laemmlli buffer or washed with PP2A phosphatase buffer and then incubated for an additional 30 minutes in the presence of 50 ng PP2A +/−10 nM okadaic acid before elution with Laemmlli buffer. Equal loading of GST-SRF protein was determined by Ponceau stain and phosphorylation of SRF was detected using a phospho-SRF S¹⁰³-specific antibody.

### Plasmid Constructs

SRE-luciferase reporter—SRE-luc was constructed by subcloning two copies of a c-fos SRF response element (TCGAC AGG ATG TCC ATA TTA GGA CAT CTG) (SEQ ID NO:3) (Treisman 1985) in an Xho I site upstream of the −36 bp rat prolactin promoter in a firefly luciferase reporter plasmid as previously described (Kapiloff et al. 1991).

−36 Prl-renilla luciferase—An oligonucleotide containing −36-+36 of the rat prolactin promoter with Bgl II and Hind III compatible ends (GATCT CGA AGG TTT ATA AAG TCA ATG TCT GCA GAT GAG AAA GCA GTG GTT CTC TTA GGA CTT CTT GGG GAA GTG TGG TC) (SEQ ID NO:4) was subcloned into pRL-null (Promega) to provide the control renilla luciferase vector.

mAKAP fragment expression vectors: pS-EGFPC1-mAKAP-1694-1833-mh adenovirus shuttle vector was constructed by subcloning a cDNA encoding a myc, His₆, and GFP-tagged mAKAP aa 1694-1833 fragment (RBD) in pEGFPC1 (Clontech) (Li, Kritzer, et al. 2013) into a pTRE shuttle vector previously modified to contain a CMV immediate early promoter. pS-EGFPC1-mh is similarly designed except lacking the mAKAP sequence. pTRE-myc-mAKAP PBD encoding a myc-tagged mAKAP aa 2134-2314 (PBD) fragment was constructed by digesting pTRE-myc-mAKAP containing a full-length, N-terminally myc-tagged mAKAP cDNA with Apa I-Sca I and ligation. pTRE-βgal encoding β-galactosidase control protein was obtained from Clontech. pAcTnTS-EGFP-mAKAP 1694-1833 mh plasmid that was used to generate AAV-RBD was constructed by subcloning a NheI-BamHI fragment of pEGFPC1-rmAKAP-1694-1833-mh (Li, Kritzer, et al. 2013) into pAcTnTs provided generously by Dr. Brent French of the University of Virginia (Prasad et al. 2011). pAcTnTs-EGFP-mh plasmid to generate AAV-GFP control virus was generated by digesting pAcTnTS-EGFP-mAKAP 1694-1833mh with Acc65I and BsRGI, blunting, and ligation. Other mAKAP plasmids were as previously described (Pare, Bauman, et al. 2005; Kapiloff, Jackson, and Airhart 2001).

SRF constructs—pFlag-SRF that expressed a Flag-tagged SRF protein was constructed by subcloning a human SRF cDNA from pCGN-SRF (Addgene Plasmid #11977) into the XbaI/EcoRI sites of the pSH160c NFATc1 expression plasmid (Ho et al. 1995). pTRE-Flag-hSRF was constructed by subcloning the Flag-tagged SRF cDNA into pTRE shuttle vector (Clontech). pTRE-3xHA-hSRF was constructed by inserting a custom sequence within the SfiI and SanDI sites of pTRE-Flag-hSRF that replaces the Flag tag with 3 tandem HA tags. S103A and S103D mutations were introduced into the pTRE plasmids by site-directed mutagenesis to introduce the sequences ATCGCTGGCAGAG (SEQ ID NO:5) and GAGCCTGGATGAA (SEQ ID NO:6) in place of GAGCCTGAGCGAG (SEQ ID NO:7). pGEX-4T1-FLAG-hSRF for expression of GST-SRF in bacteria was constructed by subcloning a NcoI (blunted)-EcoRI fragment of pTRE-Flag-hSRF into the BamHI (blunted)-EcoRI sites of pGEX-4T1.

RSK3 expression vectors: Plasmids for HA-tagged RSK3 wildtype and S218A mutant and RSK3 fragments are as previously described (Li, Kritzer, et al. 2013). pS-HA-hRSK3 1-42 adenvirus shuttle vector was constructed by subcloning a HA-tagged 1-42 cDNA into the BsaBI and NheI sites of pS-EGFPC1-mh replacing the tagged GFP cDNA.

Adenovirus were prepared using the pTRE shuttle vectors and the Adeno-X Tet-off System (Clontech) via PI-SceI and I-CeuI subcloning and purified after amplification using Vivapure AdenoPACK kits (Sartorius Stedim). These adenovirus conditionally express recombinant protein when co-infected with tetracycline transactivator-expressing virus (adeno-tTA for “tet-off” or reverse tTA for “tet-on”). Some adenovirus were constructed using a modified pTRE shuttle vector (pS) containing a constitutive CMV promoter.

### Results

Given the role of RSK3 and mAKAPβ in the determination of concentric myocyte growth, research has focused on the identification of RSK3 cardiac myocyte substrates. The transcription factor serum response factor (SRF) serves important roles in both cardiac development and adult function through the regulation of genes involved in growth and the actin cytoskeleton (Miano 2010). SRF is subject to multiple post-translational modifications (FIG. 19A), including phosphorylation at Ser¹⁰³ (Mack 2011). Because of SRF's prominent role in myocyte regulation and the previously demonstrated phosphorylation of SRF by other RSK family members (Miano 2010; Rivera et al. 1993; Janknecht et al. 1992; Hanlon, Sturgill, and Sealy 2001), SRF was considered to be an effector for RSK3 in cardiac myocytes. Phosphorylation of SRF Ser¹⁰³ by RSK3 was readily confirmed using purified glutathione-S-transferase (GST)-SRF fusion protein (data not shown). SRF contains a conserved MADS (MCM1, agamous, deficiens, SRF) domain that mediates both DNA binding to CArG box [CC(A/T)₆GG] serum response elements (SREs) and homo- and hetero-dimerization with other transcription factors (FIG. 19A). Using RSK3 small interfering nucleotides (siRNA) to deplete primary neonatal rat ventricular myocytes cultures (NRVM) of SRF by RNA interference (RNAi), it was determined that loss of RSK3 inhibited SRE-dependent transient reporter activity, including that induced by the α-adrenergic agonist phenylephrine (PE, FIG. 19B). As RSK3 binds the scaffold protein mAKAPβ (Li, Kritzer, et al. 2013), whether SRF might also be associated with mAKAPβ signalosomes, facilitating its phosphorylation was tested. Endogenous mAKAPβ was consistently co-immunoprecipitated with SRF from adult mouse heart extracts using SRF antibodies (FIG. 19C). In addition, SRF and RSK3 can associate in the presence of mAKAPβ when expressed in heterologous cells, forming ternary complexes (FIG. 19D). Accordingly, inhibition of RSK3 and mAKAPβ expression in NRVM inhibited PE-induced SRF Ser¹⁰³ phosphorylation (FIG. 19E). The isoform-specific N-terminal RSK3 domain binds a discrete “RSK3-binding domain” within mAKAPβ at residues 1694-1833 (RBD) (Li, Kritzer, et al. 2013). Expression of a myc-tagged, green fluorescent protein (GFP) RBD-fusion protein that can compete mAKAPβ-RSK3 binding (Li, Kritzer, et al. 2013) inhibited PE-induced SRF Ser¹⁰³ phosphorylation in both NRVM and primary adult rat ventricular myocyte cultures (ARVM, FIG. 19F and data not shown). Similar results were obtained by anchoring disruption using the N-terminal RSK3 peptide (data not shown). These results were corroborated in vivo. SRF Ser¹⁰³ phosphorylation was decreased in hearts obtained from both RSK3 global and mAKAPβ myocyte-specific conditional knock-out mice that were previously described (Kritzer et al. 2014; Li, Kritzer, et al. 2013), as well as in mice expressing RBD in vivo (data not shown). Together these results reveal that SRF is a RSK3 substrate in myocytes whose phosphorylation in response to catecholaminergic stimulation depends upon association with mAKAPβ signalosomes.

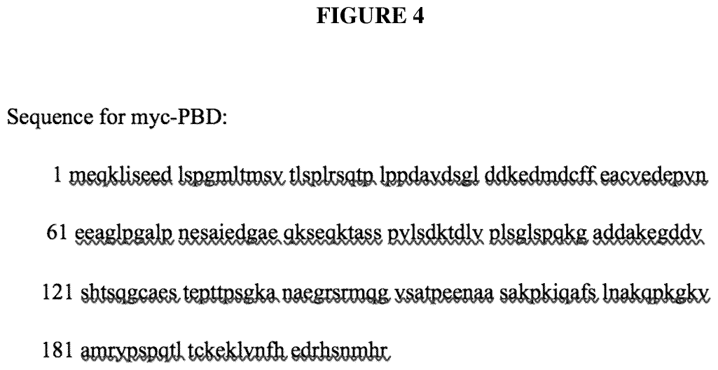

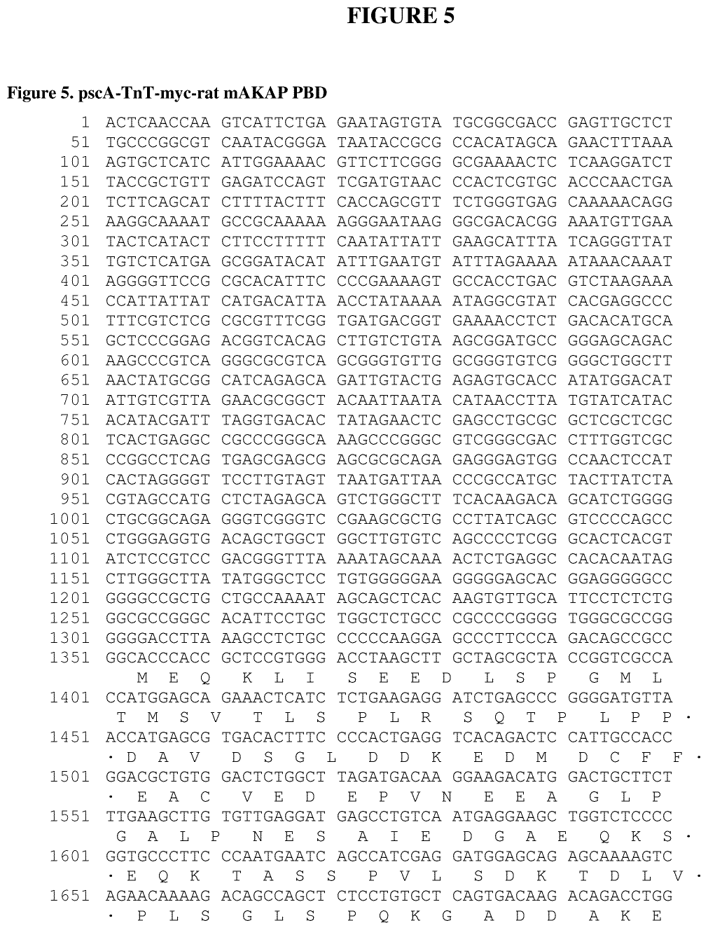

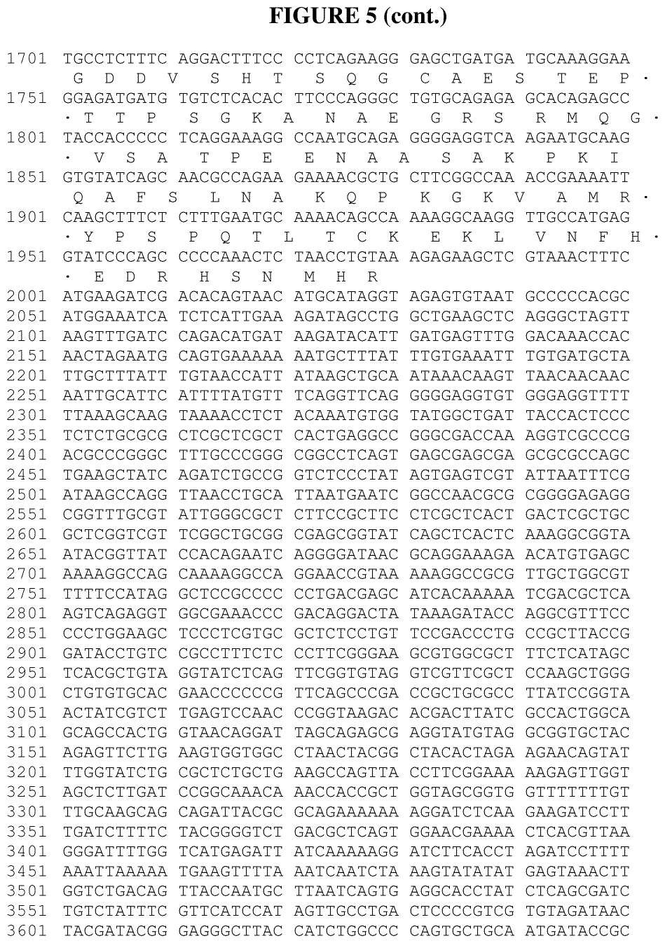

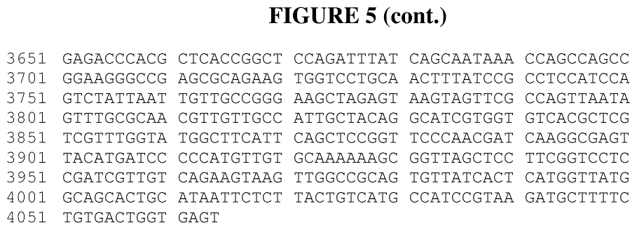

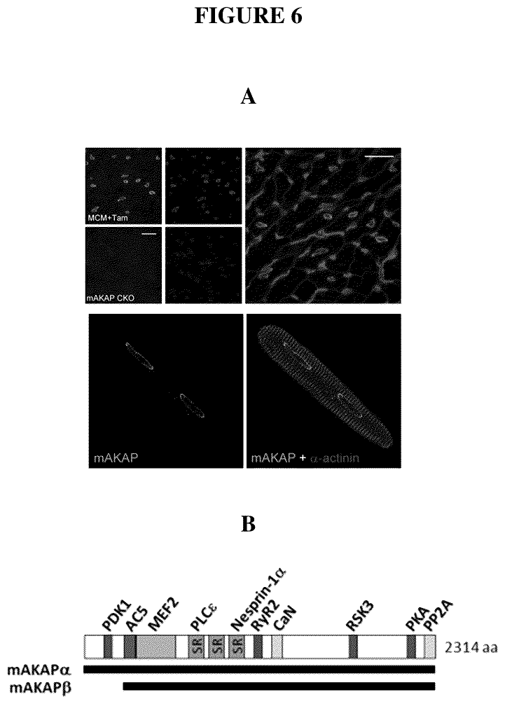

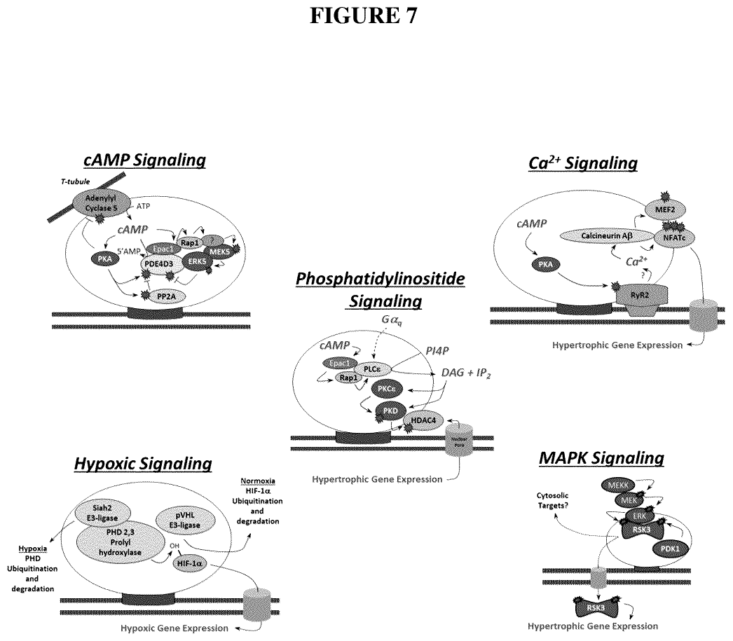

mAKAPβ binds two phosphatases, the Ca²⁺/calmodulin-dependent phosphatase calcineurin (PP2B, PPP3) and a protein kinase A (PKA)-activated isoenzyme of PP2A that contains B56δ-subunit (Dodge-Kafka et al. 2010; Li et al. 2010). Treatment of NRVM with the PP1/PP2A inhibitor okadaic acid (OA), but not the calcineurin inhibitor cyclosporin A (CsA) promoted baseline phosphorylation of SRF Ser¹⁰³ (FIG. 19G). Accordingly, purified PP2A readily dephosphorylated SRF Ser¹⁰³ (FIG. 21). Analagous to RSK3, SRF, PP2A, and mAKAPβ form ternary complexes in NRVM, as SRF and PP2A could be co-immunoprecipitated only in the presence of mAKAPβ (FIG. 19H). PP2A binds a C-terminal domain of mAKAPβ (Dodge-Kafka et al. 2010), and expression of the PP2A Binding Domain (myc-PBD, FIG. 4) competed endogenous mAKAPβ-PP2A association in myocytes (FIG. 19I). Consistent with a previously published finding that cAMP activates mAKAPβ-bound PP2A (Dodge-Kafka et al. 2010), PBD expression potentiated the induction of SRF Ser¹⁰³ phosphorylation in ARVM stimulated with the β-adrenergic isoproterenol (Iso, FIG. 19J). In aggregate, these results show that mAKAPβ signalosomes can regulate SRF Ser¹⁰³ phosphorylation in a bidirectional manner in response to different upstream stimuli.

### Example 2

### SRF Ser¹⁰³ Phosphorylation Promotes Concentric Hypertrophy

While both neonatal rat ventricular myocytes (NRVM) and adult rat ventricular myocytes (ARVM) are useful for studying molecular signaling pathways, including α-adrenergic and β-adrenergic induced hypertrophy, the two cellular preparations are significantly different in shape, ultrastructure, and in some circumstances cellular regulation (Peter, Bjerke, and Leinwand 2016). Taking advantage of their roughly cylindrical shape, ARVM was developed as an in vitro model for morphologic hypertrophy more relevant to in vivo cardiac remodeling. Characterization of the RSK3 knock-out mouse suggested that RSK3 was important for concentric hypertrophy (Passariello et al. 2016; Li, Kritzer, et al. 2013). RSK3 overexpression selectively increased the width of cultured ARVM, resulting in a significantly decreased length/width ratio (FIG. 20A,B). This result was similar to that obtained following one day of myocyte culture in the presence of the phenylephrine (PE, FIG. 20C,D). PE induced an increase of 8-10% in width and a decrease of 8-14% in length/width ratio in 24 hours, which compares favorably to the increase of 17-21% in width and the decrease of 14-21% in length/width ratio of mouse myocytes in vivo following two weeks of transverse aortic constriction (8, 16). Remarkably, expression of a SRF S103D phosphomimetic mutant also increased ARVM width, inducing concentric hypertrophy to the same degree as PE treatment. Conversely, expression of the SRF S103A mutant did not affect basal myocyte size, but inhibited the PE-induced concentric hypertrophy (FIG. 20E,F). This result was phenocopied by expression of the RBD RSK3-anchoring disruptor peptide (FIG. 20G,H) that inhibited SRF Ser¹⁰³ phosphorylation (FIG. 19F). In contrast to PE and RSK3 overexpression, chronic stimulation with the β-adrenergic agonist Iso increased both ARVM length and width, resulting in a more symmetric hypertrophy (FIG. 20I,J), similar to the effect of chronic Iso infusion in vivo (Li, Kritzer, et al. 2013). Like RBD and SRF S103A expression, displacement of PP2A phosphatase from mAKAPβ signalosomes had no effect on basal ARVM morphology. In addition, like SRF S103D expression, PBD anchoring disruptor expression did not enhance nor diminish PE-induced hypertrophy. In contrast, in the presence of Iso, PDB expression promoted ARVM concentric hypertrophy, with the Iso-induced increase in ARVM width and length tending to be greater and lesser, respectively, in the presence of PP2A displacement. This latter result was consistent with the PDB-dependent potentiation of Iso-induced SRF Ser¹⁰³ phosphorylation (FIG. 19J). Taken together, these results support a model in which mAKAPβ-anchored RSK3 and PP2A regulate SRF Ser¹⁰³ phosphorylation that promotes concentric cardiac myocyte hypertrophy.

### Example 3

Regulation of PDE4D3 by mAKAPβ-Bound PP2A

Antibodies—The following primary antibodies were used for immunoblotting: mouse monoclonal anti-GFP (Santa Cruz; 1:500), mouse monoclonal anti-VSV tag (Sigma: 1:1000), mouse monoclonal anti-mAKAP (Covance, 1:1000), 9E10 mouse anti-myc (Santa Cruz, Inc, 1:500 dilution), polyclonal anti-PP2A-C(Santa Cruz, 1:500), and polyclonal anti-PP1 catalytic subunit (Santa Cruz, Inc, 1:500). A phospho-specific antibody for phospho-PDE4D3 Ser-54 was generated and affinity purified using phosphorylated and non-phosphorylated human PDE4D3 peptides containing residues 70-81 (21st Century Biochemicals) and was used at a dilution of 1:500. Polyclonal B56δ antibodies, both non-phospho-specific and specific for phospho-Ser-566, are as previously described (Ahn et al. 2007).

Expression constructs—Expression vectors for Flag-tagged B56δ, Glutathione-S-transferase (GST) PP2A-A fusion protein, and myc- and green fluorescence protein (GFP)-tagged rat and human mAKAP are as previously described (Ahn et al. 2007; Pare, Bauman, et al. 2005; Kapiloff et al. 1999a; Kapiloff, Jackson, and Airhart 2001). The myc-tagged mAKAP construct deficient in PP2A binding was made by subcloning a cDNA fragment encoding rat mAKAP 1286-2083 generate by PCR into pCMV-Myc (Clontech). mAKAPα and mAKAPβ are two alternatively-spliced isoforms of mAKAP expressed in the heart and brain, respectively (Michel et al. 2005b). mAKAPβ is identical to mAKAPα residues 245-2314; all recombinant mAKAP proteins expressed in this paper are based on mAKAPα. The expression vector used for PDE4D3 throughout this paper was constructed by subcloning a cDNA encoding VSV-tagged PDE4D3 (Dodge et al. 2001) into a GFP-expression vector (Clontech), resulting in a double-tagged PDE4D3 protein.

Immunoprecipitation—HEK293 cells were used in this project as a heterologous system lacking mAKAP in which the various wildtype and mutant proteins could be easily expressed. Cells cultured on 60 mm plates were transfected at 50%-70% confluency by the calcium phosphate method, using 6 μg of each DNA construct per plate. Cells were harvested 24 hours after transfection in 0.5 ml HSE buffer (HEPES, pH 7.4, 150 mM NaCl, 5 mM EDTA, 1% Triton X-100 and protease inhibitors). Supernatants were incubated with 3 μg antibody and 15 μl prewashed protein A- or G-agarose beads. Following overnight incubation at 4° C., the immunoprecipitates were washed three times with the same buffer. Bound proteins were analyzed by immunoblotting.

For immunoprecipitation of endogenous, native mAKAP complexes, adult rat hearts (Pel-Freeze) were homogenized in 10 ml HSE buffer. After centrifugation at 15,000×g for 25 minutes, clarified extracts were immunoprecipitated as above.

PDE assay—PDE activity associated with immunoprecipitated protein complexes was assayed according to the method by Beavo et al. (Beavo, Bechtel, and Krebs 1974). Samples were assayed in 45 μl PDE buffer A (100 mM MOPS, pH 7.5, 4 mM EGTA, 1.0 mg/ml bovine serum albumin) and 50 μl PDE buffer B [100 mM MOPS, pH 7.5, 75 mM MgAc, 1 μM cAMP and 100,000 cpm [³H]cAMP (Dupont, NEN)]. Inhibitors were included as indicated.

Phosphatase Assay—Phosphatase activity was measured according to the method of Ahn et al. using ³²P-labeled histone as substrate (Ahn et al. 2007). Histone was radiolabeled in reactions containing 250 mM MOPS, pH 7.4, 2.5 mM MgAc, 100 mM P-mercaptoethanol, purified PKA catalytic subunit, 1 μM ATP, 20 μM histone, and 1 mCi [γ-³²P]ATP (6000 Ci/mmol). The reaction was terminated by the addition of 50% TCA, and [³²P]histone was purified from free radionucleotide by centrifugation. The [³²P]histone pellet was washed with 1 ml of ether/ethanol/HCL (4:1:0.1) once and 1 ml of ether/ethanol (4:1) three times. The substrate was then suspended in 200 μl PP2A assay buffer (25 mM Tris, pH 7.4, 1 mM DTT, and 10 mM MgCl2) before precipitation with 50% TCA. After repeated washing, the [³²P]histone was suspended in 200 μl PP2A buffer.

To measure phosphatase activity, immunoprecipitated protein complexes were washed twice in HSE buffer and once in PP2A reaction buffer. The immunoprecipitates were incubated for 30 minutes at 30° C. in 20 μl PP2A assay buffer containing 100,000 cpm [³²P]histone in the presence and absence of inhibitors. The PP2A inhibitor (Calbiochem) was used at a concentration of 30 nM. Purified I-1 was phosphorylated by PKA before using as a specific PP1 inhibitor. Reactions were terminated by the addition of 100 μl 20% TCA followed by 10 min centrifugation. TCA supernatants containing released ³²PO₄ were measured by scintillation counting.

GST-pulldowns—Glutathione resin adsorbed with PP2A-A subunit GST fusion protein or GST control protein were incubated with HEK293 cell extracts. After an overnight incubation, the beads were washed three times. Bound proteins were analyzed by immunoblotting.

Statistics—Each “n” refers to a completely independent experiment performed using separate cultures or heart preparations. All p-values were calculated using a Student's t-test.

### Results

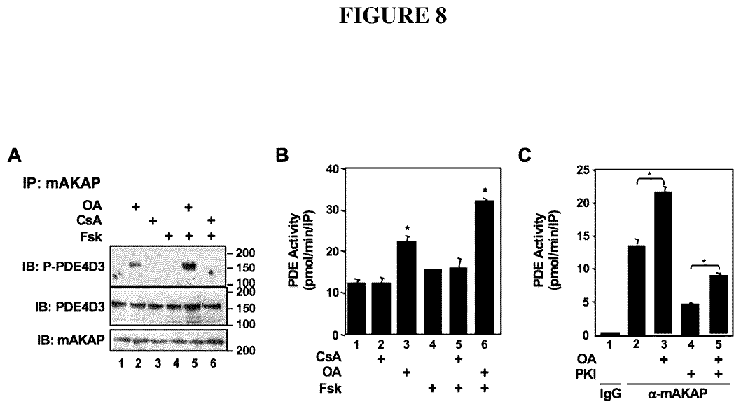

Regulation of mAKAP-bound PDE4D3 by an okadaic acid-sensitive phosphatase. A negative feedback loop intrinsic to mAKAP complexes that includes cAMP activation of PKA, PKA phosphorylation and activation of PDE4D3, and PDE4D3-catalyzed cAMP degradation has previously been described (Dodge et al. 2001). PDE4D3 phosphorylation was dependent upon PKA binding to mAKAP. Symmetrically, a mAKAP-bound phosphatase might be responsible for PDE4D3 dephosphorylation. Both PP2A and the Ca²⁺/calmodulin-dependent protein phosphatase calcineurin (PP2B) associate with the mAKAP scaffold in cardiac myocytes (Pare, Bauman, et al. 2005; Kapiloff, Jackson, and Airhart 2001; Li et al. 2009). To begin this study, a heterologous system was used to test whether PP2A or PP2B might dephosphorylate PDE4D3 at Ser-54, the residue within the PDE4D3 Upstream Conserved Region required for PKA activation (Sette and Conti 1996). HEK293 cells over-expressing mAKAP and PDE4D3 were treated with 300 μM okadaic acid (OA) to inhibit PP2A (and protein phosphatase 1 [PP1]) activity or 500 μM cyclosporin A (CsA) to inhibit PP2B activity (FIG. 8A). After immunoprecipitation of protein complexes using a mAKAP-specific antibody, PDE4D3 phosphorylation was assayed by immunoblotting with a phospho-specific antibody to residue Ser-54 had been generated. OA treatment resulted in an increase in the baseline phosphorylation of PDE4D3 Ser-54, while inhibition of PP2B had no effect (FIG. 8A, top panel, lane 2). This increased phosphorylation was further enhanced 1.8 fold when PKA was activated by the addition of the adenylyl cyclase agonist forskolin (Fsk, FIG. 8A, top panel, lane 5). Notably, forskolin alone had no significant effect in the absence of phosphatase inhibition (FIG. 8A, lane 4). Immunoblotting using a non-phospho-specific antibody for PDE4D3 and an antibody for mAKAP demonstrated that two proteins were similarly precipitated under each condition (FIG. 8A, lower panels).

As phosphorylation of PDE4D3 Ser-54 increases phosphodiesterase activity 2 fold (Sette and Conti 1996), whether OA treatment would also increase the activity of mAKAP-bound PDE4D3 was tested. mAKAP complexes were immunoprecipitated from transfected HEK293 cells and assayed for associated phosphodiesterase activity (FIG. 8B). mAKAP-associated phosphodiesterase activity in untreated cells was detected only when mAKAP was co-expressed with PDE4D3 (FIG. 8B, bar 1, and data not shown), consistent with a previous observation that PDE4D3 accounts for all of the phosphodiesterase activity associated with mAKAP in cardiac myocytes (Dodge et al. 2001). In agreement with the results obtained with the phospho-Ser-54 antibody, Fsk treatment alone was unable to significantly stimulate mAKAP-bound PDE4D3 activity in HEK293 cells, while Fsk and OA treatment together synergistically increased PDE4D3 activity (FIG. 8B, bars 3 & 6). CsA had no effect on either basal or stimulated PDE4D3 activity, suggesting that PP2B does not regulate PDE4D3 bound to mAKAP in cells under these conditions. Together, these results show that in this heterologous system, an OA-sensitive phosphatase strongly inhibits both the baseline and Fsk-stimulated phosphorylation and activity of PDE4D3 bound to mAKAP.

The enhancement of phosphodiesterase activity by OA was seen not only with expression of recombinant proteins in HEK293 cells, but also upon isolation of native mAKAP complexes from adult rat heart extracts (FIG. 8C). Both PDE4D3 and PKA are active in purified mAKAP complexes (Dodge et al. 2001). PKA activity present in endogenous mAKAP complexes is responsible for increasing phosphodiesterase activity 2-fold, as was evident upon inhibition of mAKAP-bound PKA with the specific PKA inhibitor PKI (FIG. 8C, bars 2 and 4). Importantly, OA inhibition increased mAKAP-associated phosphodiesterase activity 30% (bars 2 and 3) and 60% when PKA was also inhibited (bars 4 and 5). Taken together, these data demonstrate that an OA-sensitive phosphatase associated with the mAKAP complex is responsible for the dephosphorylation of PDE4D3 and the regulation of phosphodiesterase activity.

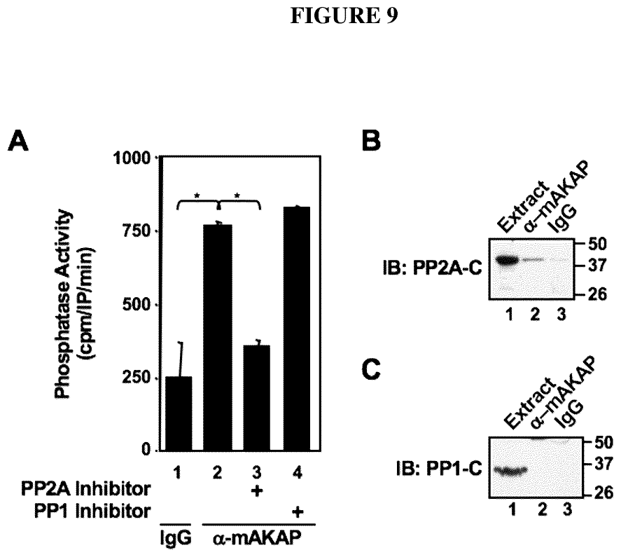

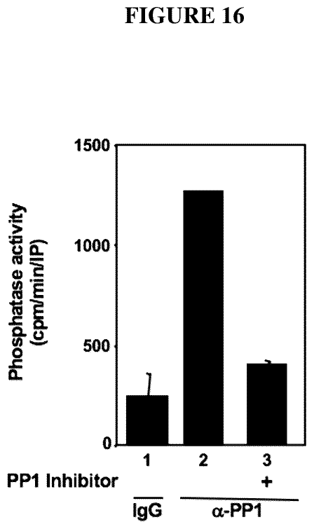

PP2A associates with the mAKAP scaffold in the heart. Having established that an OA-sensitive phosphatase was associated with the mAKAP complex, the phosphatase was identified by co-immunoprecipitation experiments. Phosphatase activity associated with mAKAP complexes isolated from heart cell extracts was measured using [³²P]histone as a substrate. There was a 3-fold enrichment of phosphatase activity over control IgG immunoprecipitates (FIG. 9A, bars 1 & 2). The mAKAP-associated phosphatase responsible for the immunoprecipitated activity was identified as PP2A, since the phosphatase activity was completely inhibited by 30 nM PP2A Inhibitor I (Li, Makkinje, and Damuni 1996), but not by addition of 100 nM PKA-phosphorylated PP1 Inhibitor-1 (Endo et al. 1996). As a positive control, the PKA-phosphorylated PP1 inhibitor-1 did inhibit PP1 isolated by immunoprecipitation with a PP1 antibody from HEK293 cell extracts (FIG. 16). The mAKAP-associated phosphatase activity was not due to mAKAP-bound PP2B, since no Ca²⁺/calmodulin was included in the phosphatase assay buffer. Confirmation of these results was obtained by immunoblot analysis of mAKAP immunoprecipitates. PP2A-C subunit, but not PP1 catalytic subunit, was detected in mAKAP-specific immunoprecipitates (FIGS. 9B & C).

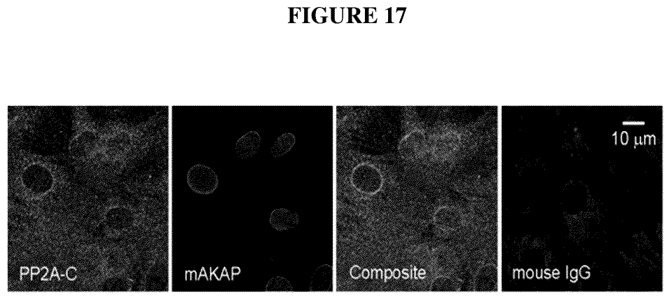

Like PKA, PP2A associates with many cellular substrates and is expected to be present in diverse intracellular compartments (Virshup 2000). Confocal fluorescent microscopy of cultured primary neonatal rat cardiomyocytes revealed that PP2A-C subunit is distributed throughout the cytoplasm in a fine punctuate pattern (FIG. 17, green). As found previously, mAKAP was localized primarily to the nuclear envelope (Pare, Easlick, et al. 2005). Consistent with the co-immunoprecipitation of mAKAP and PP2A from adult rat heart extracts, overlap of PP2A and mAKAP staining could be detected at the nuclear envelope (FIG. 17, composite image), supporting the model that a localized signaling complex consisting of discrete pools of PP2A, PKA, and PDE4D3 and the scaffold mAKAP is present in cardiac myocytes.

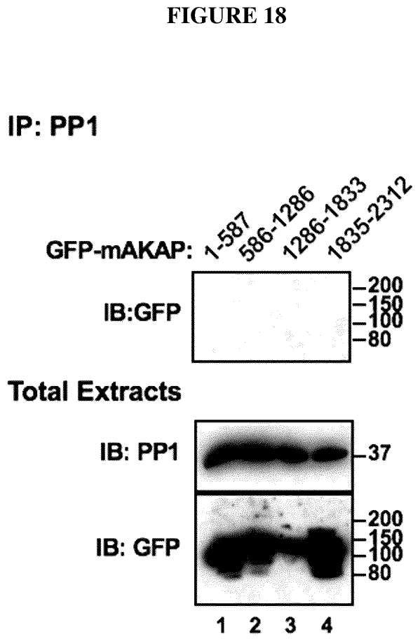

mAKAP residues 2083-2319 contain the PP2A binding domain. In order to map the PP2A binding site on mAKAP, a bacterially-expressed PP2A-A subunit GST-fusion protein was used to pull down GFP-tagged fragments of mAKAP expressed in HEK293 cells (FIGS. 10A & B). GST-PP2A-A consistently pulled down only fragments of mAKAP containing a domain C-terminal to residue 2085. Both human and rat mAKAP GFP-fusion proteins bound GST-PP2A-A, including rat mAKAP 1835-2312 and human 2085-2319. As a negative control, the GFP-mAKAP fusion proteins did not bind PP1 in HEK293 cells, consistent with the lack of co-immunoprecipitation of PP1 and mAKAP from heart extracts (FIG. 18). To confirm the mapping of the PP2A binding site on mAKAP, myc-tagged mAKAP fragments expressed in HEK293 cells were immunoprecipitated with a myc-tag antibody and assayed for associated PP2A activity (FIG. 10C). mAKAP 1286-2312, but not mAKAP 1286-2083, co-immunoprecipitated with OA-sensitive phosphatase activity. Together, these data show that PP2A binds a C-terminal site within mAKAP that is separate from the binding sites for PKA, PDE4D3, and other known mAKAP-binding proteins (FIG. 10A).

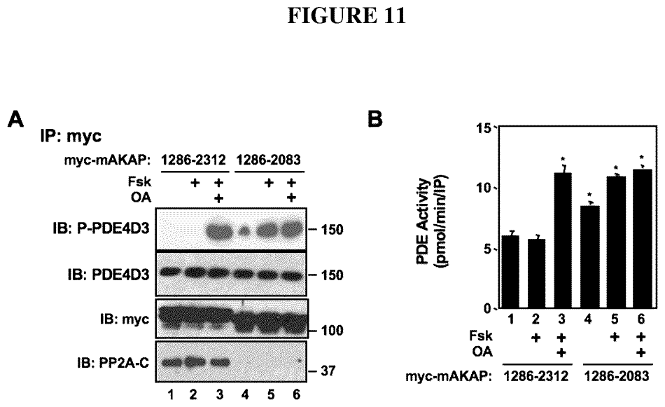

mAKAP-anchored PP2A regulates PDE4D3 phosphorylation in the complex. Data obtained using mAKAP complexes isolated from rat heart extracts implied that mAKAP-bound PP2A regulated PDE4D3 in the complex (FIG. 8C). To test whether PP2A anchoring is required for PDE4D3 dephosphorylation, PDE4D3 was expressed in HEK293 cells and a mAKAP construct containing the binding sites for PDE4D3, PKA and PP2A (myc-mAKAP 1286-2312), or a similar mAKAP construct lacking the PP2A binding site (myc-mAKAP 1286-2083). The cells were stimulated with Fsk and OA, and mAKAP complexes were subsequently isolated by immunoprecipitation. Phosphorylation of mAKAP-bound PDE4D3 was assayed by immunoblotting with the Ser-54 phospho-specific antibody. As was found upon expression of full-length mAKAP (FIG. 8A), phosphorylation of PDE4D3 bound to myc-mAKAP 1286-2312 was detected only when phosphatase activity was suppressed by OA (FIG. 11A, lane 3). Notably, upon expression of myc-mAKAP 1286-2083 which lacked significant PP2A binding (FIG. 11A, lanes 4-6), an increase in the baseline phosphorylation of mAKAP-bound PDE4D3 was detected (0.49±0.19 fold of the level obtained with OA; FIG. 11A, lanes 4 vs. 3). Moreover, upon deletion of the PP2A binding domain, Fsk alone increased phosphorylation of the phosphodiesterase to levels equivalent to that associated with PP2A-containing complexes treated with both Fsk and OA (FIG. 11A, lanes 3, 5, & 6). The changes in PDE4D3 Ser-54 phosphorylation were mirrored by changes in phosphodiesterase activity (FIG. 11B). PDE4D3 activity was 30% higher in myc-mAKAP 1286-2083 immunoprecipitates lacking PP2A than in complexes containing the phosphatase (bar 1 and 4). Importantly, no significant difference in PDE4D3 activity was seen between Fsk stimulation and Fsk stimulation in the presence of OA for the complexes lacking PP2A (bars 5 and 6). These data demonstrate the importance of PP2A anchoring for the regulation of PDE4D3 phosphorylation and activity. Furthermore, they demonstrate that PP2A serves not only to attenuate PKA-activated phosphodiesterase activity, but also to maintain a low basal level of PDE4D3 activity in unstimulated cells.

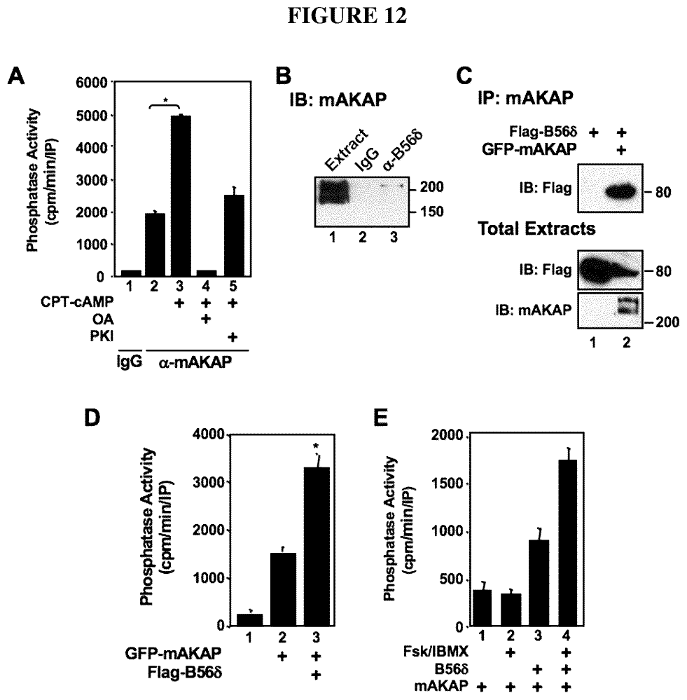

mAKAP-bound PP2A holoenzyme containing B56δsubunit is regulated by PKA. PP2A holoenzyme is composed of three subunits, including a core A and C subunit heterodimer and a B subunit that may target the holoenzyme to specific intracellular organelles (Virshup 2000). Three closely related B-subunits have been identified that are expressed in the heart and are localized to the nucleus, B56δ, B56γ1 and B56γ3 (Gigena et al. 2005; McCright et al. 1996). Recent work demonstrated PP2A holoenzyme containing B56δ is regulated by PKA phosphorylation (Ahn et al. 2007). Whether PP2A associated with mAKAP complexes might also be regulated by PKA activity was tested. Native mAKAP complexes were immunoprecipitated from adult rat heart extracts and assayed for associated phosphatase activity (FIG. 12A). mAKAP-associated phosphatase activity was increased 2.5-fold by stimulation of bound PKA with the non-hydrolysable cAMP analog CPT-cAMP (lanes 2 & 3). As controls, all immunoprecipitated phosphatase activity was inhibited by 10 nM OA (lane 4), and the CPT-cAMP-stimulated increase in phosphatase activity was blocked by the addition of the PKA inhibitor PKI (lane 5). Taken together, these data demonstrate that PP2A activity associated with mAKAP complexes in the heart is potentiated by PKA-dependent cAMP signaling.

Because mAKAP-bound PP2A was regulated by PKA activity, whether mAKAP-bound PP2A holoenzyme contained B56δ subunit was tested. Protein complexes were immunoprecipitated from adult rat heart extracts using B56δ and control (IgG) antibody (FIG. 12B). mAKAP was consistently immunoprecipitated with the B56δ antibody. In addition, Flag-tagged B56δ was expressed in HEK293 cells and showed that B56δ was immunoprecipitated with a mAKAP antibody only when co-expressed with (GFP-tagged) mAKAP (FIG. 12C). Finally, the binding of B56δ to mAKAP was shown to recruit PP2A-C subunit to the complex, because mAKAP complexes immunoprecipitated from HEK293 cell extracts were associated with greater phosphatase activity when GFP-mAKAP was co-expressed with Flag-B56δ (FIG. 12D, lanes 2 & 3). Based upon these results, B56δ recruits the PP2A-A/C core heterodimer to mAKAP complexes in the heart, conferring cAMP-dependent phosphatase activity. Accordingly, elevation of intracellular cAMP with Fsk and the phosphodiesterase inhibitor IBMX increased mAKAP-associated phosphatase activity in HEK293 cells, only when mAKAP was co-expressed with B56δ (FIG. 12E).

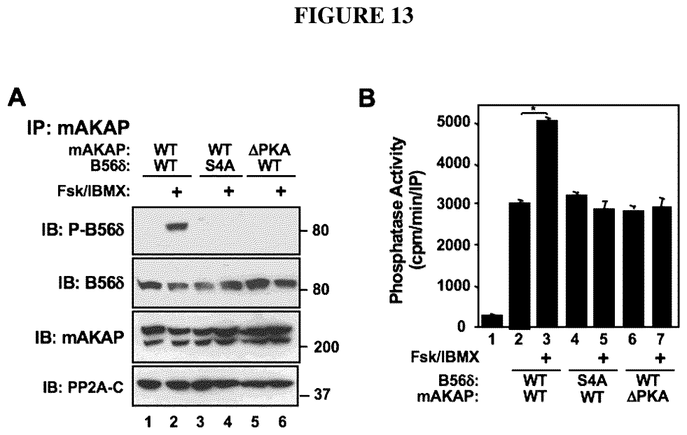

PKA Binding is required for cAMP-dependent PP2A activity in mAKAP complexes. Previous work found that PKA phosphorylates B56δ on four serine residues (53, 68, 81, 566), and Ser-566 is suggested to account for the induction of PP2A activity (Ahn et al. 2007). Since mAKAP complexes include both PKA and PP2A, association of these molecules into a complex appeared to be important for PP2A phosphorylation, just as PP2A binding to mAKAP was required for PDE4D3 de-phosphorylation (FIG. 11). To test this hypothesis, B56δ was expressed in HEK293 cells with wildtype full-length mAKAP or a full-length mAKAP mutant with an internal deletion of residues 2053-2073 comprising the PKA binding site (ΔPKA, FIG. 13A) (Pare, Bauman, et al. 2005). Following stimulation of the cells with Fsk/IBMX to elevate intracellular cAMP, mAKAP complexes were isolated by immunoprecipitation, and the phosphorylation state of B56δ was determined using a phospho-specific antibody to B56δ Ser-566 (FIG. 13A, top panel) (Ahn et al. 2007). B56δ phosphorylation was detected only after FSK/IBMX treatment and only when B56δ was co-expressed with wildtype mAKAP and not the ΔPKA mutant (FIG. 13A, lanes 2 & 6). As a control, equivalent expression of mutant and wildtype mAKAP and B56δ proteins was demonstrated by immunoblotting with non-phospho-specific antibodies (FIG. 13A, middle and bottom panels). Additionally, wildtype mAKAP was co-expressed with a mutant B56δ form containing alanine residues at each of the four PKA substrate sites (S4A). As expected, Fsk/IBMX stimulation did not induce phosphorylation of B56δ S4A (FIG. 13A lane 4). Since B56δ phosphorylation increases PP2A catalytic activity, the mAKAP-antibody immunoprecipitates were assayed for phosphatase activity (FIG. 13B). Consistent with the results obtained using the phospho-specific B56δ antibody, cAMP elevation increased phosphatase activity in mAKAP complexes 1.7 fold (FIG. 13B, lanes 2 & 3). This increase required phosphorylation of B56δ, as complexes containing the S4A mutant showed no augmentation of PP2A activity by increased cAMP (lane 5). Likewise, PKA binding to mAKAP was required to induce PP2A activity, as no increase was obtained when B56δ was co-expressed with the mAKAP ΔPKA mutant scaffold (lane 6). Interestingly, the Fsk/IBMX-induced increase in mAKAP-associated PP2A activity was not due to increased PP2A-C subunit binding to the mAKAP complexes (FIG. 13A, lanes 1 & 2). This result is in accord with an earlier suggestion that B56δ phosphorylation increases PP2A catalytic activity through conformational changes that do not affect holoenzyme formation (Ahn et al. 2007).

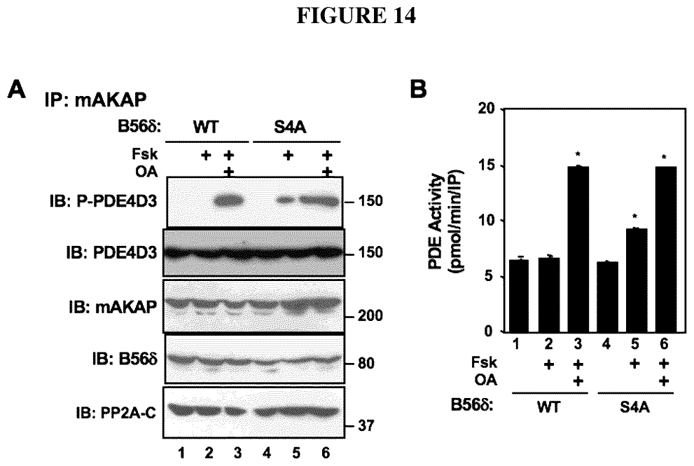

PP2A regulates PDE4D3 phosphorylation in a PKA-dependent manner. The results described above imply that PP2A dephosphorylation of PDE4D3 in B56δ-mAKAP complexes should be enhanced by PKA-catalyzed phosphorylation of the phosphatase. To address the role of B56δ phosphorylation in the regulation of PDE4D3, PDE4D3 and mAKAP were co-expressed with either wild-type B56δ or the B56δ S4A mutant that is not responsive to PKA. Cells were stimulated with Fsk before isolation of mAKAP complexes. As detected by phospho-specific antibody immunoblot and enzymatic assay, Fsk-stimulation of PDE4D3 Ser-54 phosphorylation and phosphodiesterase activity were only observed for mAKAP complexes containing wildtype B56δ when PP2A was inhibited with OA (FIGS. 14A & B, 1-3), consistent with aforementioned data (FIG. 8). In contrast, expression of B56δ S4A resulted in detectable Fsk-stimulated PDE4D3 phosphorylation (0.39±0.15 fold of Fsk/OA-stimulated cells, FIG. 14A, lane 5) and a concomitant increase in phosphodiesterase activity (FIG. 14B, lane 5), albeit not as strongly as when PP2A activity was directly inhibited by OA (FIGS. 14A & B, lanes 3 & 6). Taken together with the results shown in FIGS. 12 & 13, anchoring of a PKA-stimulated PP2A holoenzyme is responsible for the attenuation of both basal and PKA-stimulated PDE4D3 activity in the mAKAP signaling complex.

### Discussion

The results described herein define the biochemical mechanism for the dephosphorylation and inactivation of PKA-phosphorylated PDE4D3 bound by the scaffold protein mAKAP. A PP2A heterotrimer comprised of A-, C-, and B56δ-subunits binds a C-terminal site on mAKAP distinct from the binding sites for other known mAKAP partners (FIG. 10). The association of PP2A with the mAKAP scaffold is of functional significance in two important and novel ways. First, by binding both PP2A and PDE4D3, mAKAP sequesters the phosphatase in close proximity to the phosphodiesterase, allowing for efficient PDE4D3 de-phosphorylation and down-regulation (FIG. 11). Second, by binding both PKA and PP2A, mAKAP promotes cAMP-dependent phosphorylation of the PP2A B56δ subunit and induction of PP2A activity (FIG. 13). The relevance of multimolecular signaling complex formation was evident upon expression of mAKAP mutants lacking binding sites for PP2A and PKA.

The concept of phosphatase targeting to generate substrate specificity was first proposed in the mid-1980's with the identification of the glycogen-particle-associated protein as the first PP1-targeting subunit (Bauman and Scott 2002). Since this initial observation, several other phosphatase targeting motifs have been determined (Virshup 2000). AKAPs represent an important mechanism to link phosphatases with their appropriate substrates, and several AKAPs bind protein phosphatases. It has been recently published that mAKAP binds PP2B (calcineurin), and that this interaction is important for PP2B-dependent NFATc3 activation in myocytes (Li et al. 2009). However, PP2B binding to mAKAP does not appear to regulate PDE4D3, as inhibition of PP2B did not affect PDE4D3 Ser-54 phosphorylation or phosphodiesterase activity (FIG. 8). The present data support a unique role for PP2A bound to mAKAP in dephosphorylation of the phosphodiesterase and, as a result, in the control of local cAMP levels.

The overall role of phosphatases in regulating cellular cAMP concentration has yet to be fully explored. In rat adipocytes, PP2A was found to regulate both PDE3B activity and phosphorylation (Resjo et al. 1999). In addition to being phosphorylated by PKA on Ser-54, PDE4D3 is phosphorylated on Ser-579 by MAP kinases, including by ERK5 present in mAKAP complexes (Hoffmann et al. 1999; Dodge-Kafka et al. 2005). Although PP1 does not appear to bind mAKAP (FIG. 9 and FIG. 18), PP1 may dephosphorylate PDE4D3 Ser-579 in other cellular domains, since the addition of purified PP1 to isolated PDE4D3 decreased phosphorylation at this site. Phosphatase(s) are also responsible for the dephosphorylation of mAKAP-bound PDE4D3 at Ser-579, as well as the second PKA site on PDE4D3, Ser-16 (Carlisle Michel et al. 2004).

The anchoring hypothesis suggests that AKAPs function to target the actions of PKA towards specific substrates by localizing both proteins to the same signaling complex. Herein is demonstrated a new target for PKA in the mAKAP complex, the PP2A B56δ-subunit. Previous work found phosphorylation of B56δ stimulated PP2A activity and enhanced de-phosphorylation of DARPP-32 (Ahn et al. 2007). In accordance with these results, stimulation of cardiac myocytes with β-adrenergic receptor agonists increases PP2A activity (De Arcangelis, Soto, and Xiang 2008). The mAKAP scaffold may facilitate this event, as the association of the anchoring protein with both PKA and PP2A is important for the cAMP-enhanced increase in phosphatase activity (FIGS. 11 & 13). Hence, mAKAP has a role in the regulation of phosphatase activity in the heart.

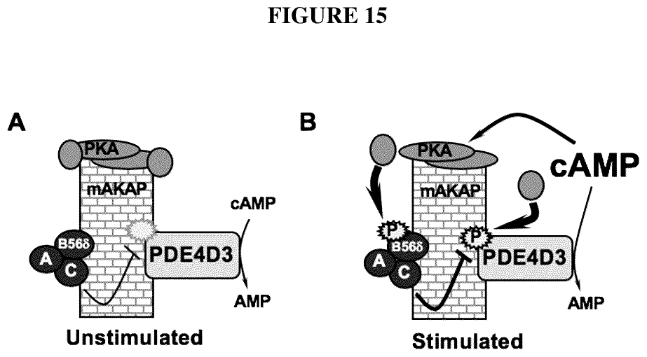

Based upon these results, a model is proposed in which PP2A serves a dual role in regulating cAMP levels near mAKAP signaling complexes (FIG. 15). First, PP2A in mAKAP complexes should maintain PDE4D3 in a dephosphorylated, minimally active state in the absence of GPCR stimulation (FIG. 15A), presumably allowing for a more rapid rise in cAMP levels in response to agonist. Second, following induction of activating cAMP levels by GPCR stimulation, PKA will phosphorylate both PDE4D3 and PP2A (FIG. 15B). In contrast to the negative feedback on cAMP levels mediated by enhanced PDE4D3 phosphorylation, PKA phosphorylation of PP2A opposes PDE4D3 activation. By inhibiting PDE4D3 phosphorylation, PP2A presumably potentiates and prolongs the actions of local cAMP as part of a positive feedback loop. Thus, in conjunction with the potential inhibition of PDE4D3 by mAKAP-bound ERK5 that has been previously described (not illustrated) (Dodge-Kafka et al. 2005), the mAKAP signaling complex is poised to finely regulate local cAMP levels both by multiple feedback loops intrinsic to the complex, as well as by crosstalk with upstream MAPK signaling pathways. It has been observed that PP2A expression and intracellular localization are altered in heart failure (Reiken et al. 2001; Ai and Pogwizd 2005). Whether PP2A-mediated positive feedback or PDE4D3-mediated negative feedback predominately controls cAMP levels local to mAKAP complexes may ultimately depend both on the stoichiometry of PP2A binding to mAKAP and the relative rates of PDE4D3 phosphorylation and dephosphorylation by PKA and PP2A in disease states.

The present examples demonstrate a novel mechanism by which the scaffold protein mAKAP maintains dynamic regulation of anchored PDE4D3 activity through the association with PDE4D3, PKA and PP2A. Each of the three enzymes plays an important role in the temporal control of cAMP concentration in the vicinity of perinuclear mAKAP complex. This intricate regulation of local cAMP by the mAKAP “signalosome” represents a broader role for AKAPs and phosphatase in the control of cAMP compartmentation.

### Example 4

Use of PBD as a treatment for HFrEF

Heart failure, the common end-stage for cardiac disease, is a syndrome of major public health significance, affecting 6.5 million Americans, including 960,000 new cases each year (Benjamin et al. 2017). Symptomatic heart failure patients can be divided almost evenly into those with reduced (HFrEF) and those with preserved ejection fraction. First-line therapy for heart failure includes angiotensin-converting enzyme (ACE) inhibitors and β-adrenergic receptor blockers (β-blockers) that at least for HFrEF can improve survival and quality of life, as well as reduce mortality (Ponikowski et al. 2016). Despite these and other adjunct therapies, however, 5-year mortality remains about 50% for heart failure (39% in a 2016 post-myocardial infarction study) (Benjamin et al. 2017; Gerber et al. 2016), necessitating the discovery of new therapeutic approaches. Phosphorylation of SRF represents a novel mechanism regulating the transition from compensated hypertrophy to the dilated, failing heart in HFrEF.

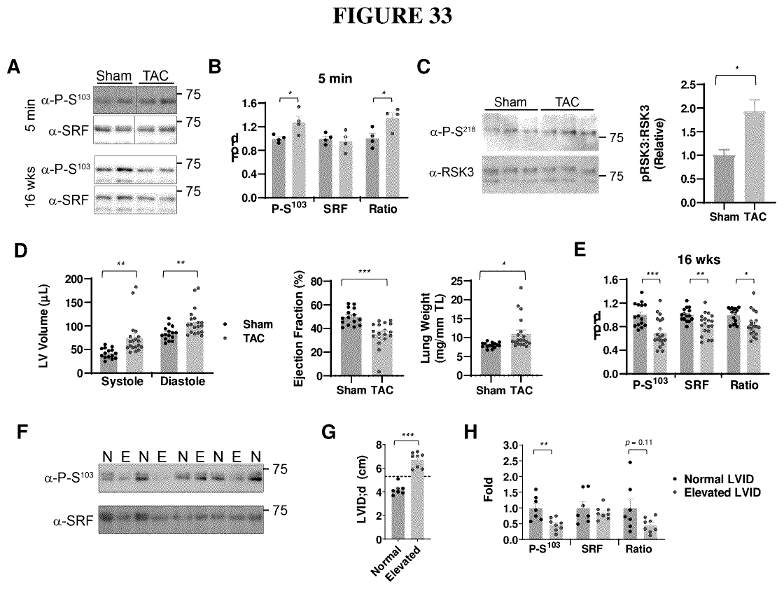

As discussed above, expression of SRF S103D both in vitro and in vivo will promote concentric myocyte hypertrophy. In addition, expression of the PP2A anchoring disruptor PBD attenuated the eccentric hypertrophy induced by Iso-treatment of cultured adult myocytes (FIG. 20). These results suggest that SRF S¹⁰³ phosphorylation drives growth in width, while attenuating any elongation of the cardiac myocyte. Given these results and the association of SRF dephosphorylation with systolic dysfunction induced by long term pressure overload (FIG. 33A, E), restoration of normal or increased SRF phosphorylation will prevent the ventricular dilatation resulting in HFrEF in diseases of chronic pressure overload and ischemic heart disease.

Mechanisms that induce “compensatory” concentric hypertrophy early in heart disease predispose the heart to later systolic dysfunction and eventual failure (Schiattarella and Hill 2015). In this regard, targeting of RSK3-mAKAPβ complexes will attenuate cardiac remodeling due to pressure overload and prevent heart failure (Kritzer et al. 2014; Li, Kritzer, et al. 2013). While inhibition of signaling pathways that induce remodeling, including concentric hypertrophy, may be desirable early in disease, the question remains whether efforts to maintain signals promoting concentric and attenuating eccentric myocyte hypertrophy would preserve cardiac volumes and contractility when initiated when the heart is at a stage in the disease process characterized by the eccentric growth and ventricular dilatation leading to HFrEF. Accordingly, maintaining SRF phosphorylation is a strategy to block the eccentric changes in ventricular morphology that typify end-stage disease and HFrEF. The fact that maintaining SRF phosphorylation is a strategy to block the eccentric changes in ventricular morphology that typify end-stage disease and HFrEF is further supported by new observations by the present inventors that SRF phosphorylation is increased in mice subjected to acute pressure overload and reduced in mice and humans undergoing ventricular dilation. Phosphorylated SRF was increased 28% in total left ventricular extracts (which includes about one-third myocytes by cell number) within 5 minutes after induction of pressure overload (FIG. 33 A, B), when RSK3 activation, as detected by S²¹⁸ phosphorylation, was increased 1.9-fold (FIG. 33 C). Remarkably, 16 weeks after transverse aortic constriction surgery, when the hearts were dilated and the mice were in heart failure (FIG. 33 D), phosphorylated SRF was suppressed 30% below that present in sham-operated controls (FIG. 33E). These results are consistent with a phosphatase being responsible for dephosphorylating SRF during the induction of eccentric hypertrophy, opposing RSK3-catalyzed phosphorylation. The relevance of these findings to human disease was assessed using patient tissue samples. When compared to SRF Ser¹⁰³ phosphorylation in left ventricular tissue from patients with normal left ventricular interior diameter, SRF Ser¹⁰³ phosphorylation in patients with dilated hearts was reduced 53% (p=0.005, FIG. 33F-H).

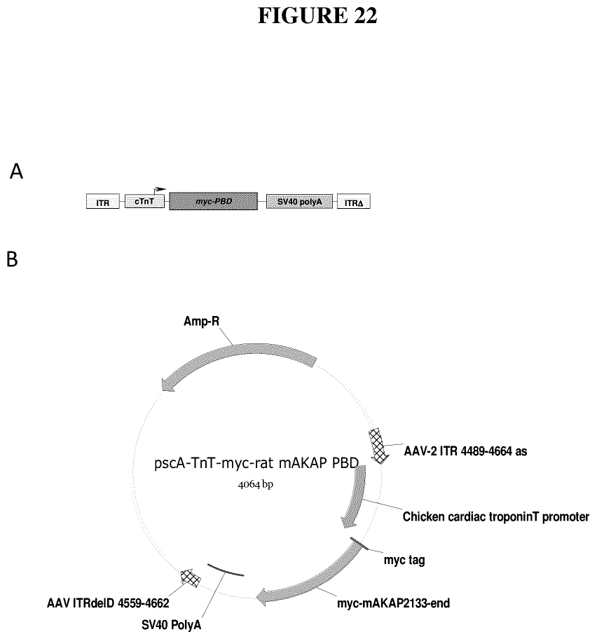

Improved ventricular geometry, i.e., decreased LV internal diameters due to less elongated myocytes and/or increased LV wall thickness due to wider myocytes, will decrease wall stress (Law of LaPlace) and improve systolic function in the heart prone to HFrEF. The prevention of systolic dysfunction has been obtained for a new AAV gene therapy vector based upon expression of the mAKAPβ-derived PBD (FIG. 22).

Treatment of Myocardial Infarction. Coronary heart disease is a leading cause of HFrEF (Writing Group et al. 2016). 8-week old C57BL/6 WT mice were subjected to permanent LAD ligation or sham thoracotomy. Two days post-operatively, heart function was evaluated by echocardiography and the mice were randomized by EF and body weight (FIG. 23B). Two cohorts of mice to be treated with either AAVsc.myc-PBD (n=8) or AAVsc.GFP (n=5) were defined that had average ejection fraction=34% 2-days after LAD ligation (FIG. 23D). Mice were injected via the tail vein 3 days post-operatively with 5×10¹¹ vg. While control GFP mice exhibited progressively decreased ejection fraction (EF to 21%), PBD mice exhibited long term restoration of systolic function (EF at 8 weeks post-operatively=43%; p<0.0001). In addition, AAVsc.myc-PBD treated mice had reduced left ventricular volumes consistent with improved cardiac function (systole—69 μl for PBD vs 156 μl for GFP, p<0.001; diastole—118 μl vs.192 μl; p<0.001). At end-point, gravimetrically, ventricular and atrial hypertrophy were reduced (p=0.053 and 0.024, respectively, indexed to tibial length, FIG. 23C), and pulmonary edema, a sign of heart failure, tended to be improved (p=0.078). These results demonstrate that PP2A anchoring disruptor therapy, that displaces PP2A from mAKAPβ where it can dephosphorylate SRF, constitutes a novel therapeutic approach for the prevention of heart failure with reduced ejection fraction in ischemic heart disease.

### Methods:

General Method for Ligation of the Left Coronary Artery: The mice were anesthetized with 5% isoflurane for induction and then 2.5-3% for maintenance. Orotracheal intubation was performed using a 16G catheter, and the mouse then ventilated mechanically using a minivent ventilator. The skin over the site of left lateral thoracotomy was prepped and draped in sterile fashion using providone-iodine 10% solution. A heating pad was used to keep mice warm during procedures to prevent heat loss. Surgically sterile non-medicated ophthalmic ointment was applied to the eyes preoperatively to prevent corneal drying. Surgery was performed under microscope view. Once adequate sedation was achieved, the chest was opened via left lateral thoracotomy at the fourth intercostal space. If muscle bleeding was present, hemostasis was achieved by the using a thermal cauterizer (e.g. fine tip Bovie). A 3 mm retractor was used to separate the ribs. Following pericardiotomy, the left coronary artery was ligated with a 7-0 prolene suture to produce an anterior MI. The chest was closed in 3 layers with 5-0 absorbable suture (muscle) and silk 6-0 (for 2 ligatures in the ribs and for the skin). Buprenorphine slow release (Bup-SR-LAB) 0.5-1 mg/kg s.c. was administered in a single dose immediately after surgery to control pain for 72 hr. Fluid replacement was administered immediately after surgery (e.g. Sterile saline solution 0.9%, IP). The mice were allowed to recover until alert and active. Sham-operated mice that experience all but the placement of the coronary artery ligature served as controls.

Echocardiography: Mice minimally anesthetized with 1-2% isoflurane were studied using a Vevo 2100®, High-Resolution Imaging System (VisualSonics). M-mode images were obtained for mice under anesthesia at various time-points. Posterior wall and anterior wall diastolic and systolic thicknesses and left ventricular cavity end-diastolic (LVEDD) and end-systolic diameters (LVESD) were measured, permitting estimation of LV volumes, fractional shortening and ejection fraction.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

### REFERENCES

Using siRNA for gene silencing is a rapidly evolving tool in molecular biology, ThermoFisher Scientific, retrieved Jun. 16, 2017 <https://http://www.thermofisher.com/us/en/home/references/ambion-tech-support/rnai-sirna/general-articles/-sirna-design-guidelines.html>.
Abrenica B, AlShaaban M, Czubryt M P. The A-kinase anchor protein AKAP121 is a negative regulator of cardiomyocyte hypertrophy. J Mol Cell Cardiol 46: 674-681, 2009.
Ahn J H, McAvoy T, Rakhilin S V, Nishi A, Greengard P, Nairn A C (2007) Protein kinase A activates protein phosphatase 2A by phosphorylation of the B56delta subunit. Proc Natl Acad Sci USA 104:2979-2984.
Ai X, Pogwizd S M (2005) Connexin 43 downregulation and dephosphorylation in nonischemic heart failure is associated with enhanced colocalized protein phosphatase type 2A. Circ Res 96:54-63.
Andino L M, Conlon T J, Porvasnik S L, Boye S L, Hauswirth W W, Lewin A S (2007) Rapid, widespread transduction of the murine myocardium using self-complementary Adeno-associated virus. Genetic vaccines and therapy 5:13.
Anjum R, Blenis J. The RSK family of kinases: emerging roles in cellular signalling. Nat Rev Mol Cell Biol. 2008; 9(10):747-758.
Appert-Collin A, Cotecchia S, Nenniger-Tosato M, Pedrazzini T, Diviani D. The A-kinase anchoring protein (AKAP)-Lbc-signaling complex mediates alpha1 adrenergic receptor-induced cardiomyocyte hypertrophy. Proc Natl Acad Sci USA 104: 10140-10145, 2007.
Avkiran M, Cook A R, Cuello F. Targeting Na+/H+ exchanger regulation for cardiac protection: a RSKy approach?Curr Opin Pharmacol. 2008; 8:133-140.
Bain J, Plater L, Elliott M, Shpiro N, Hastie C J, McLauchlan H, Klevernic I, Arthur J S, Alessi D R, Cohen P. The selectivity of protein kinase inhibitors: a further update. Biochem J. 2007; 408:297-315.
Backs J, Worst B C, Lehmann L H, Patrick D M, Jebessa Z, Kreusser M M, Sun Q, Chen L, Heft C, Katus H A, Olson E N (2011) Selective repression of MEF2 activity by PKA-dependent proteolysis of HDAC4. J Cell Biol 195:403-415.
Bauman A L, Scott J D (2002) Kinase- and phosphatase-anchoring proteins: harnessing the dynamic duo. Nat Cell Biol 4:E203-206.
Bauman A L, Michel J J, Henson E, Dodge-Kafka K L, Kapiloff M S, “The mAKAP signalosome and cardiac myocyte hypertrophy,” IUBMB Life. 2007 March; 59(3):163-9. Review.
Beavo J A, Bechtel P J, Krebs E G (1974) Preparation of homogeneous cyclic AMP-dependent protein kinase(s) and its subunits from rabbit skeletal muscle. Methods Enzymol 38:299-308.
Beene D L, Scott J D. A-kinase anchoring proteins take shape. Curr Opin Cell Biol 19: 192-198, 2007.
Benjamin E J et al. (2017) Heart Disease and Stroke Statistics-2017 Update: A Report From the American Heart Association. Circulation 135:e146-e603.
Benjamin E J et al. (2019) Heart Disease and Stroke Statistics-2019 Update: A Report From the American Heart Association. Circulation 139: e56-e528.
Bers D M (2006) Cardiac ryanodine-receptor phosphorylation: target sites and functional consequences. Biochem J 396:el-3.
Bers D M. Calcium cycling and signaling in cardiac myocytes. Annu Rev Physiol 70: 23-49, 2008.
Bione S, Maestrini E, Rivella S, Mancini M, Regis S, Romeo G, Toniolo D (1994) Identification of a novel X-linked gene responsible for Emery-Dreifuss muscular dystrophy. Nat Genet 8:323-327.
Black B L, Olson E N (1998) Transcriptional control of muscle development by myocyte enhancer factor-2 (MEF2) proteins. Annu Rev Cell Dev Biol 14:167-196.
Bonne G, Di Barletta M R, Vamous S, Becane H M, Hammouda E H, Merlini L, Muntoni F, Greenberg C R, Gary F, Urtizberea J A, Duboc D, Fardeau M, Toniolo D, Schwartz K (1999) Mutations in the gene encoding lamin N C cause autosomal dominant Emery-Dreifuss muscular dystrophy. Nat Genet 21:285-288.
Bourajjaj M, Armand A S, da Costa Martins P A, Weijts B, van der Nagel R, Heeneman S, Wehrens X H, De Windt L J (2008) NFATc2 is a necessary mediator of calcineurin-dependent cardiac hypertrophy and heart failure. J Biol Chem 283:22295-22303.
Brown J H, Del Re D P, Sussman M A. The Rac and Rho hall of fame: a decade of hypertrophic signaling hits. Circ Res 98: 730-742, 2006.
Burns-Hamuro L L, Ma Y, Kammerer S, Reineke U, Self C, Cook C, Designing isoform-specific peptide disruptors of protein kinase A localization. Proc Natl Acad Sci USA. 2003 Apr. 1; 100(7):4072-7.
Brunton L L, Hayes J S, Mayer S E (1979) Hormonally specific phosphorylation of cardiac troponin I and activation of glycogen phosphorylase. Nature 280:78-80.
Buck M, Chojkier M. C/EBPbeta-Thr217 phosphorylation signaling contributes to the development of lung injury and fibrosis in mice. PLoS One. 2011; 6(10):e25497.
Bueno O F, Wilkins B J, Tymitz K M, Glascock B J, Kimball T F, Lorenz J N, Molkentin J D (2002) Impaired cardiac hypertrophic response in Calcineurin Abeta-deficient mice. Proc Natl Acad Sci USA 99:4586-4591.
Bueno O F, Lips D J, Kaiser R A Wilkins B J, Dai Y S, Glascock B J, Klevitsky R, Hewett T E, Kimball T R, Aronow B J, Doevendans P A, Molkentin J D (2004) Calcineurin Abeta gene targeting predisposes the myocardium to acute ischemia-induced apoptosis and dysfunction. Circ Res 94:91-99.
Burchfield J S, Xie M, Hill J A (2013) Pathological ventricular remodeling: mechanisms: part 1 of 2. Circulation 128:388-400.
Cappola T P. Molecular remodeling in human heart failure. J Am Coll Cardiol 51: 137-138, 2008.
Cariolato L, Cavin S, Diviani D. A-kinase anchoring protein (AKAP)-Lbc anchors a PKN-based signaling complex involved in alpha1-adrenergic receptor-induced p38 activation. J Biol Chem 286: 7925-7937, 2011.
Carlisle Michel J J, Dodge K L, Wong W, Mayer N C, Langeberg L K, Scott J D (2004) PKA-phosphorylation of PDE4D3 facilitates recruitment of the mAKAP signalling complex. Biochem J 381:587-592.
Carlucci A, Lignitto L, Feliciello A. Control of mitochondria dynamics and oxidative metabolism by cAMP, AKAPs and the proteasome. Trends Cell Biol 18: 604-613, 2008.
Carnegie G K, Smith F D, McConnachie G, Langeberg L K, Scott J D. AKAP-Lbc nucleates a protein kinase D activation scaffold. Mol Cell 15: 889-899, 2004.
Carnegie G K, Soughayer J, Smith F D, Pedroja B S, Zhang F, Diviani D, Bristow M R, Kunkel M T, Newton A C, Langeberg L K, Scott J D. AKAP-Lbc mobilizes a cardiac hypertrophy signaling pathway. Mol Cell 32: 169-179, 2008.
Chaturvedi D, Poppleton H M, Stringfield T, Barbier A, Patel T B. Subcellular localization and biological actions of activated RSK1 are determined by its interactions with subunits of cyclic AMP-dependent protein kinase. Mol Cell Biol. 2006; 26:4586-4600.
Chen L, Kurokawa J, Kass R S. Phosphorylation of the A-kinase anchoring protein Yotiao contributes to protein kinase A regulation of a heart potassium channel. J Biol Chem 280: 31347-31352, 2005.
Chen L, Kurokawa J, Kass R S. Phosphorylation of the A-kinase-anchoring protein Yotiao contributes to protein kinase A regulation of a heart potassium channel. J Biol Chem 280: 31347-31352, 2005.
Chen L, Marquardt M L, Tester D J, Sampson K J, Ackerman M J, Kass R S. Mutation of an A-kinase-anchoring protein causes long-Q T syndrome. Proc Natl Acad Sci USA 104: 20990-20995, 2007.
Chen P P, Patel J R, Rybakova I N, Walker J W, Moss R L. Protein kinase A-induced myofilament desensitization to Ca²⁺ as a result of phosphorylation of cardiac myosin-binding protein C. J Gen Physiol 136: 615-627, 2010.
Christian F, Szaszak M, Friedl S, Drewianka S, Lorenz D, Goncalves A, Furkert J, Vargas C, Schmieder P, Gotz F, Zuhlke K, Moutty M, Gottert H, Joshi M, Reif B, Haase H, Morano I, Grossmann S, Klukovits A, Verli J, Gaspar R, Noack C, Bergmann M, Kass R, Hampel K, Kashin D, Genieser H G, Herberg F W, Willoughby D, Cooper D M, Baillie G S, Houslay M D, von Kries J P, Zimmermann B, Rosenthal W, Klussmann E. Small molecule AKAP-protein kinase A (PKA) interaction disruptors that activate PKA interfere with compartmentalized cAMP signaling in cardiac myocytes. J Biol Chem 286: 9079-9096, 2011.
Clerk A, Cullingford T E, Fuller S J, Giraldo A, Markou T, Pikkarainen S, Sugden P H (2007) Signaling pathways mediating cardiac myocyte gene expression in physiological and stress responses. J Cell Physiol 212:311-322.
Consensus (1987). “Effects of enalapril on mortality in severe congestive heart failure. Results of the Cooperative North Scandinavian Enalapril Survival Study (CONSENSUS).” N Engl J Med 316(23): 1429-1435.
Cuello F, Snabaitis A K, Cohen M S, Taunton J, Avkiran M. Evidence for direct regulation of myocardial Na+/H+ exchanger isoform 1 phosphorylation and activity by 90-kDa ribosomal S6 kinase (RSK): effects of the novel and specific RSK inhibitor fink on responses to alpha1-adrenergic stimulation. Mol Pharmacol. 2007; 71:799-806.
De Arcangelis V, Soto D, Xiang Y (2008) Phosphodiesterase 4 and phosphatase 2A differentially regulate cAMP/protein kinase a signaling for cardiac myocyte contraction under stimulation of betal adrenergicreceptor. Mol Pharmacol 74:1453-1462.
Diviani D, Abuin L, Cotecchia S, Pansier L. Anchoring of both PKA and 14-3-3 inhibits the Rho-GEF activity of the AKAP-Lbc signaling complex. EMBO J 23: 2811-2820, 2004.
Diviani D, Dodge-Kafka K L, Li J, Kapiloff M S. A-kinase anchoring proteins: scaffolding proteins in the heart,” Am J Physiol Heart Circ Physiol. 2011 November; 301(5):H1742-53.
Dobrev D, Wehrens X H (2014) Role of RyR2 phosphorylation in heart failure and arrhythmias: Controversies around ryanodine receptor phosphorylation in cardiac disease. Circ Res 114:1311-1319; discussion 1319.
Dodge-Kafka, K. L., M. Gildart, J. Li, H. Thakur, and M. S. Kapiloff. 2018. ‘Bidirectional regulation of HDAC5 by mAKAPbeta signalosomes in cardiac myocytes’, Journal of Molecular and Cellular Cardiology, 118: 13-25.
Dodge-Kafka, K. L., A. Bauman, N. Mayer, E. Henson, L. Heredia, J. Ahn, T. McAvoy, A. C. Nairn and M. S. Kapiloff (2010). “cAMP-stimulated protein phosphatase 2A activity associated with muscle A kinase-anchoring protein (mAKAP) signaling complexes inhibits the phosphorylation and activity of the cAMP-specific phosphodiesterase PDE4D3.” J Biol Chem 285(15): 11078-11086.
Dodge-Kafka, K. L. and M. S. Kapiloff (2006). “The mAKAP signaling complex: integration of cAMP, calcium, and MAP kinase signaling pathways.” Eur J Cell Biol 85(7): 593-602.
Dodge-Kafka, K. L., J. Soughayer, G. C. Pare, J. J. Carlisle Michel, L. K. Langeberg, M. S. Kapiloff and J. D. Scott (2005). “The protein kinase A anchoring protein mAKAP coordinates two integrated cAMP effector pathways.” Nature 437(7058): 574-578.
Dodge, K. L., S. Khouangsathiene, M. S. Kapiloff, R. Mouton, E. V. Hill, M. D. Houslay, L. K. Langeberg and J. D. Scott (2001). “mAKAP assembles a protein kinase A/PDE4 phosphodiesterase cAMP signaling module.” EMBO J 20(8): 1921-1930.
Diviani D, Soderling J, Scott J D. AKAP-Lbc anchors protein kinase A and nucleates Galpha 12-selective Rho-mediated stress fiber formation. J Biol Chem 276: 44247-44257, 2001.
Dodge K L, Khouangsathiene S, Kapiloff M S, Mouton R, Hill E V, Houslay M D, Langeberg L K, Scott J D. mAKAP assembles a protein kinase A/PDE4 phosphodiesterase cAMP signaling module. EMBO J 20: 1921-1930, 2001.
Dodge-Kafka K L, Bauman A, Kapiloff M S, A-kinase anchoring proteins as the basis for cAMP signaling,” Handb Exp Pharmacol. 2008; (186):3-14.
Dodge-Kafka K L, Bauman A, Mayer N, Henson E, Heredia L, Ahn J, McAvoy T, Nairn A C, Kapiloff M S. cAMP-stimulated protein phosphatase 2A activity associated with muscle A kinase-anchoring protein (mAKAP) signaling complexes inhibits the phosphorylation and activity of the cAMP-specific phosphodiesterase PDE4D3. J Biol Chem. 2010; 285:11078-11086.
Dodge-Kafka K L, Kapiloff M S, “The mAKAP signaling complex: integration of cAMP, calcium, and MAP kinase signaling pathways,” Eur J Cell Biol. 2006 July; 85(7):593-602. Epub 2006 Feb. 7. Review.
Dodge-Kafka K L, Langeberg L, Scott J D (2006) Compartmentation of cyclic nucleotide signaling in the heart: the role of A-kinase anchoring proteins. Circ Res 98:993-1001.
duBell W H, Lederer W J, Rogers T B (1996) Dynamic modulation of excitation-contraction coupling by protein phosphatases in rat ventricular myocytes. J Physiol 493 (Pt 3):793-800.
duBell W H, Gigena M S, Guatimosim S, Long X, Lederer W J, Rogers T B (2002) Effects of PP1/PP2A inhibitor calyculin A on the E-C coupling cascade in murine ventricular myocytes. Am J Physiol Heart Circ Physiol 282:H38-48.
Dulhunty A F, Beard N A, Pouliquin P, Casarotto Bond M. AKAP-mediated targeting of protein kinase a regulates contractility in cardiac myocytes. Circ Res 88: 291-297, 2001. Fischmeister R, Castro L R, Abi-Gerges A, Rochais F, Jurevicius J, Leroy J,
Dummler B A, Hauge C, Silber J, Yntema H G, Kruse L S, Kofoed B, Hemmings B A, Alessi D R, Frodin M. Functional characterization of human RSK4, a new 90-kDa ribosomal S6 kinase, reveals constitutive activation in most cell types. J Biol Chem. 2005; 280:13304-13314
Edgley A J, Krum H, Kelly D J. Targeting fibrosis for the treatment of heart failure: a role for transforming growth factor-beta. Cardiovasc Ther. 2012; 30(1):e30-40.
Eide T, Coghlan V, Orstavik S, Holsve C, Solberg R, Skalhegg B S, Lamb N J, Langeberg L, Fernandez A, Scott J D, Jahnsen T, Tasken K. Molecular cloning, chromosomal localization, and cell cycle-dependent subcellular distribution of the A-kinase anchoring protein, AKAP95. Exp Cell Res 238: 305-316, 1998.
Elbashir S M, Martinez J, Patkaniowska A, Lendeckel W, Tuschl T, Functional anatomy of SiRNAs for mediating efficient RNAi in Drosophila melanogaster embryo lysate, The EMBO Journal, Vol. 20, No. 23, pp. 6877-6888, 2001.
Endo S, Zhou X, Connor J, Wang B, Shenolikar S (1996) Multiple structural elements define the specificity of recombinant human inhibitor-1 as a protein phosphatase-1 inhibitor. Biochemistry 35:5220-5228.
Escobar M, Cardenas C, Colavita K, Petrenko N B, Franzini-Armstrong C. Structural evidence for perinuclear calcium microdomains in cardiac myocytes. J Mol Cell Cardiol 50: 451-459, 2011.
Fabiato A. Calcium-induced release of calcium from the cardiac sarco-plasmic reticulum. Am J Physiol Cell Physiol 245: C1-C14, 1983.
Farah C S, Reinach F C. The troponin complex and regulation of muscle contraction. FASEB J 9: 755-767, 1995.
Fatkin D, MacRae C, Sasaki T, Wolff M R, Porcu M, Frenneaux M, Atherton J, Vidaillet H J, Jr., Spudich S, De Girolami U, Seidman J G, Seidman C, Muntoni F, Muehle G, Johnson W, McDonough B (1999) Missense mutations in the rod domain of the lamin N C gene as causes of dilated cardiomyopathy and conduction-system disease. N Engl J Med 341:1715-1724.
Faul C, Dhume A, Schecter A D, Mundel P. Protein kinase A, Ca2+/calmodulin-dependent kinase II, and calcineurin regulate the intracellular trafficking of myopodin between the Z-disc and the nucleus of cardiac myocytes. Mol Cell Biol 27: 8215-8227, 2007.
Fink M A, Zakhary D R, Mackey J A, Desnoyer R W, Apperson-Hansen C, Damron D S, Bond M. AKAP-mediated targeting of protein kinase a regulates contractility in cardiac myocytes. Circ Res 88: 291-297, 2001. Fischmeister R, Castro L R, Abi-Gerges A, Rochais F, Jurevicius J, Leroy J, Vandecasteele G (2006) Compartmentation of cyclic nucleotide signaling in the heart: the role of cyclic nucleotide phosphodiesterases. Circ Res 99:816-828.
Fodstad H, Swan H, Laitinen P, Piippo K, Paavonen K, Viitasalo M, Toivonen L, Kontula K. Four potassium channel mutations account for 73% of the genetic spectrum underlying long-Q T syndrome (LQTS) and provide evidence for a strong founder effect in Finland. Ann Med 36, Suppl 1: 53-63, 2004.
Francis S H, Corbin J D. Structure and function of cyclic nuleotide-dependent protein kinases. Annu Rev Physiol 56: 237-272, 1994.
Fraser I D, Tavalin S J, Lester L B, Langeberg L K, Westphal A M, Dean R A, Marrion N V, Scott J D. A novel lipid-anchored A-kinase anchoring protein facilitates cAMP-responsive membrane events. EMBO J 17: 2261-2272, 1998.
Frey N, Katus H A, Olson E N, Hill J A. Hypertrophy of the heart: a new therapeutic target?Circulation 109: 1580-1589, 2004.
Friday B B, Mitchell P O, Kegley K M, Pavlath G K (2003) Calcineurin initiates skeletal muscle differentiation by activating MEF2 and MyoD. Differentiation 71:217-227.
Fuller M D, Emrick M A, Sadilek M, Scheuer T, Catterall W A. Molecular mechanism of calcium channel regulation in the fight-or-flight response. Sci Signal 3: ra70, 2010.
Gaffin R D, Pena J R, Alves M S, Dias F A, Chowdhury S A, Heinrich L S, Goldspink P H, Kranias E G, Wieczorek D F, Wolska B M. Long-term rescue of a familial hypertrophic cardiomyopathy caused by a mutation in the thin filament protein, tropomyosin, via modulation of a calcium cycling protein. J. Mol. Cell. Cardiol. 2011.
Gao T, Yatani A, Dell'Acqua M L, Sako H, Green S A, Dascal N, Scott J D, Hosey M M. cAMP-dependent regulation of cardiac L-type Ca2+ channels requires membrane targeting of PKA and phosphorylation of channel subunits. Neuron 19: 185-196, 1997.
Gao Y, Dickerson J B, Guo F, Zheng J, Zheng Y. Rational design and characterization of a Rac GTPase-specific small molecule inhibitor. Proc Natl Acad Sci USA 101: 7618-7623, 2004.
Gelb B D, Tartaglia M. RAS signaling pathway mutations and hypertrophic cardiomyopathy: getting into and out of the thick of it. J Clin Invest. 2011; 121:844-847.
Gentilucci L, Tolomelli A, Squassabia F, Peptides and peptidomimetics in medicine, surgery and biotechnology. Curr Med Chem 13: 2449-2466, 2006.
Gerber, Y., S. A. Weston, M. Enriquez-Sarano, C. Berardi, A. M. Chamberlain, S. M. Manemann, R. Jiang, S. M. Dunlay and V. L. Roger (2016). “Mortality Associated With Heart Failure After Myocardial Infarction: A Contemporary Community Perspective.” Circ Heart Fail 9(1): e002460.
Gigena M S, Ito A, Nojima H, Rogers T B (2005) A B56 regulatory subunit of protein phosphatase 2A localizes to nuclear speckles in cardiomyocytes. Am J Physiol Heart Circ Physiol 289:H285-294.
Go A S et al. (2014) Heart disease and stroke statistics—2014 update: a report from the American Heart Association. Circulation 129:e28-e292.
Gold M G, Lygren B, Dokurno P, Hoshi N, McConnachie G, Tasken K, Carlson C R, Scott J D, Barford D. Molecular basis of AKAP specificity for PKA regulatory subunits. Mol Cell 24: 383-395, 2006.
Goldschmidt-Clermont P J, Seo D M, Wang L, Beecham G W, Liu Z J, Vazquez-Padron R I, Dong C, Hare J M, Kapiloff M S, Bishopric N H, Pericak-Vance M, Vance J M, Velazquez O C, “Inflammation, stem cells and atherosclerosis genetics,” Curr Opin Mol Ther. 2010 December; 12(6):712-23. Review.
Good M C, Zalatan J G, Lim W A. Scaffold proteins: hubs for controlling the flow of cellular information. Science. 2011; 332:680-686.
Gould K L, Bretscher A, Esch F S, Hunter T. cDNA cloning and sequencing of the protein-tyrosine kinase substrate, ezrin, reveals homology to band 4.1. EMBO J 8: 4133-4142, 1989. Gray P C, Scott J D, Catterall W A. Regulation of ion channels by cAMP-dependent protein kinase and A-kinase anchoring proteins. Curr Opin Neurobiol 8: 330-334, 1998.
Grossman W, Jones D, McLaurin L P (1975) Wall stress and patterns of hypertrophy in the human left ventricle. J Clin Invest 56:56-64.
Group, Consensus Trial Study. 1987. ‘Effects of enalapril on mortality in severe congestive heart failure. Results of the Cooperative North Scandinavian Enalapril Survival Study (CONSENSUS)’, New England Journal of Medicine, 316: 1429-35.
Guo T, Cornea R L, Huke S, Camors E, Yang Y, Picht E, Fruen B R, Bers D M. Kinetics of FKBP12.6 binding to ryanodine receptors in permeabilized cardiac myocytes and effects on Ca sparks. Circ Res 106: 1743-1752, 2010.
Guo, H., B. Liu, L. Hou, E. The, G. Li, D. Wang, Q. Jie, W. Che and Y. Wei (2015). “The role of mAKAPbeta in the process of cardiomyocyte hypertrophy induced by angiotensin II.” Int J Mol Med 35(5): 1159-1168.
Hagemann D, Xiao R P. Dual site phospholamban phosphorylation and its physiological relevance in the heart. Trends Cardiovasc Med 12: 51-56, 2002.
Hanks S K, Quinn A M, Hunter T. The protein kinase family: conserved features and deduced phylogeny of the catalytic domains. Science. 1988; 241:42-52.
Hanlon M, Sturgill T W, Sealy L (2001) ERK2- and p90(Rsk2)-dependent pathways regulate the CCAAT/enhancer-binding protein-beta interaction with serum response factor. J Biol Chem 276:38449-38456.
Harada H, Becknell B, Wilm M, Mann M, Huang L J, Taylor S S, Scott J D, Korsmeyer S J. Phosphorylation and inactivation of BAD by mitochondria-anchored protein kinase A. Mol Cell 3: 413-422, 1999.
Hayes J S, Brunton L L, Mayer S E (1980) Selective activation of particulate cAMP-dependent protein kinase by isoproterenol and prostaglandin El. J Biol Chem 255:5113-5119.
Heidenreich, P. A., N. M. Albert, L. A. Allen, D. A. Bluemke, J. Butler, G. C. Fonarow, J. S. Ikonomidis, O. Khavjou, M. A. Konstam, T. M. Maddox, G. Nichol, M. Pham, I. L. Pina, J. G. Trogdon, C. American Heart Association Advocacy Coordinating, T. Council on Arteriosclerosis, B. Vascular, R. Council on Cardiovascular, Intervention, C. Council on Clinical, E. Council on, Prevention and C. Stroke (2013). “Forecasting the impact of heart failure in the United States: a policy statement from the American Heart Association.” Circ Heart Fail 6(3): 606-619.
Heineke J, Molkentin J D (2006) Regulation of cardiac hypertrophy by intracellular signaling pathways. Nat Rev Mol Cell Biol 7:589-600.
Hell J W. Beta-adrenergic regulation of the L-type Ca²⁺ channel CaV1.2 by PKA rekindles excitement. Sci Signal 3: pe33, 2010.
Henn V, Edemir B, Stefan E, Wiesner B, Lorenz D, Theilig F, Schmitt R, Vossebein L, Tamma G, Beyermann M, Krause E, Herberg F W, Valenti G, Bachmann S, Rosenthal W, Klussmann E. Identification of a novel A-kinase anchoring protein 18 isoform and evidence for its role in the vasopressin-induced aquaporin-2 shuttle in renal principal cells. J Biol Chem 279: 26654-26665, 2004.
Hill J A, Olson E N. Cardiac plasticity. N Engl J Med 358: 1370-1380, 2008.
Ho S N, Thomas D J, Timmerman L A, Li X, Francke U, Crabtree G R (1995) NFATc3, a lymphoid-specific NFATc family member that is calcium-regulated and exhibits distinct DNA binding specificity. J Biol Chem 270:19898-19907.
Hoffmann R, Baillie G S, MacKenzie S J, Yarwood S J, Houslay M D (1999) The MAP kinase ERK2 inhibits the cyclic AMP-specific phosphodiesterase HSPDE4D3 by phosphorylating it at Ser579. EMBO J 18:893-903.
Houser S R (2014) Role of RyR2 phosphorylation in heart failure and arrhythmias: protein kinase A-mediated hyperphosphorylation of the ryanodine receptor at serine 2808 does not alter cardiac contractility or cause heart failure and arrhythmias. Circ Res 114:1320-1327; discussion 1327.
Huang L J, Durick K, Weiner J A, Chun J, Taylor S S. D-AKAP2, a novel protein kinase A anchoring protein with a putative RGS domain. Proc Natl Acad Sci USA 94: 11184-11189, 1997.
Huang L J, Durick K, Weiner J A, Chun J, Taylor S S. Identification of a novel dual specificity protein kinase A anchoring protein, D-AKAP1. J Biol Chem 272: 8057-8064, 1997.
Huang L J, Durick K, Weiner J A, Chun J, Taylor S S. Identification of a novel protein kinase A anchoring protein that binds both type I and type II regulatory subunits. J Biol Chem. 1997; 272:8057-8064.
Hulme J T, Ahn M, Hauschka S D, Scheuer T, Catterall W A. A novel leucine zipper targets AKAP15 and cyclic AMP-dependent protein kinase to the C terminus of the skeletal muscle Ca2 channel and modulates its function. J Biol Chem 277: 4079-4087, 2002.
Hulme J T, Lin T W, Westenbroek R E, Scheuer T, Catterall W A. Beta-adrenergic regulation requires direct anchoring of PKA to cardiac CaV1.2 channels via a leucine zipper interaction with A kinase-anchoring protein 15. Proc Natl Acad Sci USA 100: 13093-13098, 2003.
Hulme J T, Westenbroek R E, Scheuer T, Catterall W A. Phosphorylation of serine 1928 in the distal C-terminal domain of cardiac CaV1.2 channels during betal-adrenergic regulation. Proc Natl Acad Sci USA 103: 16574-16579, 2006.
Hundsrucker C, Klussmann E. Direct AKAP-mediated protein-protein interactions as potential drug targets. Hand Exp Pharmacol 186: 483-503, 2008.
Hundsrucker C, Krause G, Beyermann M, Prinz A, Zimmermann B, Diekmann 0, Lorenz D, Stefan E, Nedvetsky P, Dathe M, Christian F, McSorley T, Krause E, McConnachie G, Herberg F W, Scott J D, Rosenthal W, Klussmann E. High-affinity AKAP7delta-protein kinase A interaction yields novel protein kinase A-anchoring disruptor peptides. Biochem J 396: 297-306, 2006.
Jaakkola P, Mole D R, Tian Y M, Wilson M I, Gielbert J, Gaskell S J, Kriegsheim A, Hebestreit H F, Mukherji M, Schofield C J, Maxwell P H, Pugh C W, Ratcliffe P J. Targeting of HIF-alpha to the von Hippel-Lindau ubiquitylation complex by O₂-regulated prolyl hydroxylation. Science 292: 468-472, 2001.
Janknecht R, Hipskind R A, Houthaeve T, Nordheim A, Stunnenberg H G (1992) Identification of multiple SRF N-terminal phosphorylation sites affecting DNA binding properties. EMBO J 11:1045-1054.
Jugdutt B I (2003) Remodeling of the myocardium and potential targets in the collagen degradation and synthesis pathways. Curr Drug Targets Cardiovasc Haematol Disord 3:1-30.
Kamisago M, Sharma S D, DePalma S R, Solomon S, Sharma P, McDonough B, Smoot L, Mullen M P, Woolf P K, Wigle E D, Seidman J G, Seidman C E. Mutations in sarcomere protein genes as a cause of dilated cardiomyopathy. N Engl J Med 343: 1688-1696, 2000.
Kammerer S, Burns-Hamuro L L, Ma Y, Hamon S C, Canaves J M, Shi M M, Nelson M R, Sing C F, Cantor C R, Taylor S S, Braun A. Amino acid variant in the kinase binding domain of dual-specific A kinase-anchoring protein 2: a disease susceptibility polymorphism. Proc Natl Acad Sci USA 100: 4066-4071, 2003.
Kapiloff M S, Chandrasekhar K D, “A-kinase anchoring proteins: temporal and spatial regulation of intracellular signal transduction in the cardiovascular system,” Journal Cardiovasc Pharmacol. 2011 October; 58(4):337-8.
Kapiloff M S, Jackson N, Airhart N. mAKAP and the ryanodine receptor are part of a multi-component signaling complex on the cardiomyocyte nuclear envelope. J Cell Sci 114: 3167-3176, 2001.
Kapiloff M S, Piggott L A, Sadana R, Li J, Heredia L A, Henson E, Efendiev R, Dessauer C W, “An adenylyl cyclase-mAKAPbeta signaling complex regulates cAMP levels in cardiac myocytes,” J Biol Chem. 2009 Aug. 28; 284(35):23540-6.
Kapiloff M S, Mathis J M, Nelson C A, Lin C R, Rosenfeld M G (1991) Calcium/calmodulin-dependent protein kinase mediates a pathway for transcriptional regulation. Proc Natl Acad Sci USA 88:3710-3714.
Kapiloff M S, Schillace R V, Westphal A M, Scott J D. mAKAP: an A-kinase anchoring protein targeted to the nuclear membrane of differentiated myocytes. J Cell Sci 112: 2725-2736, 1999.
Kato Y, Zhao M, Morikawa A, Sugiyama T, Chakravortty D, Koide N, Yoshida T, Tapping R I, Yang Y, Yokochi T, Lee J D (2000) Big mitogen-activated kinase regulates multiple members of the MEF2 protein family. J Biol Chem 275:18534-18540.
Keely S L (1977) Activation of cAMP-dependent protein kinase without a corresponding increase in phosphorylase activity. Res Commun Chem Pathol Pharmacol 18:283-290.
Keely S L (1979) Prostaglandin El activation of heart cAMP-dependent protein kinase: apparent dissociation of protein kinase activation from increases in phosphorylase activity and contractile force. Mol Pharmacol 15:235-245.
Kehat I, Davis J, Tiburcy M, Accornero F, Saba-El-Leil M K, Maillet M, York A J, Lorenz J N, Zimmermann W H, Meloche S, Molkentin J D. Extracellular signal-regulated kinases 1 and 2 regulate the balance between eccentric and concentric cardiac growth. Circ Res. 2011; 108:176-183.
Kehat I, Molkentin J D. Molecular pathways underlying cardiac re-modeling during pathophysiological stimulation. Circulation. 2010; 122:2727-2735.
Kentish J C, McCloskey D T, Layland J, Palmer S, Leiden J M, Martin A F, Solaro R J. Phosphorylation of troponin I by protein kinase A accelerates relaxation and crossbridge cycle kinetics in mouse ventricular muscle. Circ Res 88: 1059-1065, 2001.
Kido M, Du L, Sullivan C C, Li X, Deutsch R, Jamieson S W, Thistlethwaite P A. Hypoxia-inducible factor 1-alpha reduces infarction and attenuates progression of cardiac dysfunction after myocardial infarction in the mouse. J Am Coll Cardiol 46: 2116-2124, 2005.
Kim Y, Phan D, van Rooij E, Wang D Z, McAnally J, Qi X, Richardson J A, Hill J A, Bassel-Duby R, Olson E N (2008) The MEF2D transcription factor mediates stress-dependent cardiac remodeling in mice. J Clin Invest 118:124-132.
Kimura T E, Jin J, Zi M, Prehar S, Liu W, Oceandy D, Abe J, Neyses L, Weston A H, Cartwright E J, Wang X. Targeted deletion of the extracellular signal-regulated protein kinase 5 attenuates hypertrophic response and promotes pressure overload-induced apoptosis in the heart. Circ Res. 2010; 106:961-970.
Kinderman F S, Kim C, von Daake S, Ma Y, Pham B Q, Spraggon G, Xuong N H, Jennings P A, Taylor S S. A dynamic mechanism for AKAP binding to RII isoforms of cAMP-dependent protein kinase. Mol Cell 24: 397-408, 2006.
Klussmann E, Edemir B, Pepperle B, Tamma G, Henn V, Klauschenz E, Hundsrucker C, Maric K, Rosenthal W. Ht31: the first protein kinase A anchoring protein to integrate protein kinase A and Rho signaling. FEBS Lett 507: 264-268, 2001.
Kodama H, Fukuda K, Pan J, Sano M, Takahashi T, Kato T, Makino S, Manabe T, Murata M, Ogawa S. Significance of ERK cascade compared with JAK/STAT and PI3-K pathway in gp130-mediated cardiac hypertrophy. Am J Physiol Heart Circ Physiol. 2000; 279(4):H1635-1644.
Kontaridis M I, Yang W, Bence K K, Cullen D, Wang B, Bodyak N, Ke Q, Hinek A, Kang P M, Liao R, Neel B G. Deletion of Ptpn11 (Shp2) in cardiomyocytes causes dilated cardiomyopathy via effects on the extracellular signal-regulated kinase/mitogen-activated protein kinase and RhoA signaling pathways. Circulation. 2008; 117:1423-1435.
Kritzer M D, Li J, Dodge-Kafka K, Kapiloff M S, “AKAPs: the architectural underpinnings of local cAMP signaling,” J Mol Cell Cardiol. 2012 February; 52(2):351-8.
Kritzer, M. D., J. Li, C. L. Passariello, M. Gayanilo, H. Thakur, J. Dayan, K. Dodge-Kafka and M. S. Kapiloff (2014). “The scaffold protein muscle A-kinase anchoring protein beta orchestrates cardiac myocyte hypertrophic signaling required for the development of heart failure.” Circ Heart Fail 7(4): 663-672.
Kumar, D., T. A. Hacker, J. Buck, L. F. Whitesell, E. H. Kaji, P. S. Douglas and T. J. Kamp (2005). “Distinct mouse coronary anatomy and myocardial infarction consequent to ligation.” Coron Artery Dis 16(1): 41-44.
Lacana E, Maceyka M, Milstien S, Spiegel S. Cloning and characterization of a protein kinase A anchoring protein (AKAP)-related protein that interacts with and regulates sphingosine kinase 1 activity. J Biol Chem 277: 32947-32953, 2002.
Layland J, Solaro R J, Shah A M. Regulation of cardiac contractile function by troponin I phosphorylation. Cardiovasc Res 66: 12-21, 2005.
Lechward K, Awotunde O S, Swiatek W, Muszynska G (2001) Protein phosphatase 2A: variety of forms and diversity of functions. Acta Biochim Pol 48:921-933.
Lehnart, S. E., X. H. Wehrens, S. Reiken, S. Warrier, A. E. Belevych, R. D. Harvey, W. Richter, S. L. Jin, M. Conti and A. R. Marks (2005). “Phosphodiesterase 4D deficiency in the ryanodine-receptor complex promotes heart failure and arrhythmias.” Cell 123(1): 25-35.
Lester L B, Langeberg L K, Scott J D. Anchoring of protein kinase A facilitates hormone-mediated insulin secretion. Proc Natl Acad Sci USA 94: 14942-14947, 1997.
Li C L, Sathyamurthy A, Oldenborg A, Tank D, Ramanan N (2014) SRF phosphorylation by glycogen synthase kinase-3 promotes axon growth in hippocampal neurons. J Neurosci 34:4027-4042.
Li H, Adamik R, Pacheco-Rodriguez G, Moss J, Vaughan M. Protein kinase A-anchoring (AKAP) domains in brefeldin A-inhibited guanine nucleotide-exchange protein 2 (BIG2). Proc Natl Acad Sci USA 100: 1627-1632, 2003.
Li J, Kritzer M D, Michel J J, Le A, Thakur H, Gayanilo M, Passariello C L, Negro A, Danial J B, Oskouei B, Sanders M, Hare J M, Hanauer A, Dodge-Kafka K, Kapiloff M S, “Anchored p90 ribosomal S6 kinase 3 is required for cardiac myocyte hypertrophy,” Circ Res. 2013 Jan. 4; 112(1):128-39.
Li J, Negro A, Lopez J, Bauman A L, Henson E, Dodge-Kafka K, Kapiloff M S. The mAKAPbeta scaffold regulates cardiac myocyte hypertrophy via recruitment of activated calcineurin. J Mol Cell Cardiol 48: 387-394, 2010.
Li J, Negro A, Lopez J, Bauman A L, Henson E, Dodge-Kafka K, Kapiloff M S, “The mAKAPbeta scaffold regulates cardiac myocyte hypertrophy via recruitment of activated calcineurin,” J Mol Cell Cardiol. 2010 February; 48(2):387-94.
Li J, Vargas M A, Kapiloff M S, Dodge-Kafka K L, Regulation of MEF2 transcriptional activity by calcineurin/mAKAP complexes,” Exp Cell Res. 2013 Feb. 15; 319(4):447-54.
Li, J., S. Aponte Paris, H. Thakur, M. S. Kapiloff, and K. L. Dodge-Kafka. 2019. ‘Muscle A-kinase-anchoring protein-beta-bound calcineurin toggles active and repressive transcriptional complexes of myocyte enhancer factor 2D’, Journal of Biological Chemistry, 294: 2543-54.
Li M, Makkinje A, Damuni Z (1996) Molecular identification of I1PP2A, a novel potent heat-stable inhibitor protein of protein phosphatase 2A. Biochemistry 35:6998-7002.
Liu Q, Hofmann P A (2004) Protein phosphatase 2A-mediated cross-talk between p38 MAPK and ERK in apopfosis of cardiac myocytes. Am J Physiol Heart Circ Physiol 286:H2204-2212.
Lohse M J, Engelhardt S, Eschenhagen T. What is the role of beta-adrenergic signaling in heart failure?Circ Res 93: 896-906, 2003.
Lu J T, Kass R S. Recent progress in congenital long Q T syndrome. Curr Opin Cardiol 25: 216-221, 2010.
Lygren B, Carlson C R, Santamaria K, Lissandron V, McSorley T, Lorenz D, Wiesner B, Rosenthal W, Zaccolo M, Tasken K, Klussmann E. AKAP-complex regulates the Ca²⁺ reuptake into heart sarcoplasmic reticulum. EMBO Rep 8: 1061-1067, 2007.
Lygren B, Tasken K. The potential use of AKAP18delta as a drug target in heart failure patients. Expert Opin Biol Ther 8: 1099-1108, 2008.
Mack C P (2011) Signaling mechanisms that regulate smooth muscle cell differentiation. Arterioscler Thromb Vase Biol 31:1495-1505.
Mackenzie K F, Topping E C, Bugaj-Gaweda B, Deng C, Cheung Y F, Olsen A E, Stockard C R, High Mitchell L, Baillie G S, Grizzle W E, De Vivo M, Houslay M D, Wang D, Bolger G B
(2008) Human PDE4A8, a novel brain-expressed PDE4 cAMP-specific phosphodiesterase that has undergone rapid evolutionary change. Biochem J 411:361-369.
MacKenzie S J, Baillie G S, McPhee I, Bolger G B, Houslay M D (2000) ERK2 mitogen-activated protein kinase binding, phosphorylation, and regulation of the PDE4D cAMP-specific phosphodiesterases. The involvement of COOH-terminal docking sites and NH2-terminal UCR regions. J Biol Chem 275:16609-16617.
Maloney D J, Hecht S M. Synthesis of a potent and selective inhibitor of p90 Rsk. Org Lett. 2005; 7:1097-1099.
Maron B J, Maron M S. Hypertrophic cardiomyopathy. Lancet. 2013; 381(9862):242-255.
Maruyama Y, Nishida M, Sugimoto Y, Tanabe S, Turner J H, Kozasa T, Wada T, Nagao T, Kurose H. Galpha(12/13) mediates alpha(1)-adrenergic receptor-induced cardiac hypertrophy. Circ Res 91: 961-969, 2002.
Martinez, E. C., C. L. Passariello, J. Li, C. J. Matheson, K. Dodge-Kafka, P. Reigan and M. S. Kapiloff (2015). “RSK3: A regulator of pathological cardiac remodeling.” IUBMB Life 67(5): 331-337.
Marx S O, Kurokawa J, Reiken S, Motoike H, D′Armiento J, Marks A R, Kass R S. Requirement of a macromolecular signaling complex for beta adrenergic receptor modulation of the KCNQ1-KCNE1 potassium channel. Science 295: 496-499, 2002.
Marx S O, Reiken S, Hisamatsu Y, Gaburjakova M, Gaburjakova J, Yang Y M, Rosemblit N, Marks A R. Phosphorylation-dependent regulation of ryanodine receptors: a novel role for leucine/isoleucine zippers. J Cell Biol. 2001; 153:699-708.
Marx S O, Reiken S, Hisamatsu Y, Jayaraman T, Burkhoff D, Rosemblit N, Marks A R. PKA phosphorylation dissociates FKBP12.6 from the calcium release channel (ryanodine receptor): defective regulation in failing hearts. Cell 101: 365-376, 2000.
Maxwell P H, Wiesener M S, Chang G W, Clifford S C, Vaux E C, Cockman M E, Wykoff C C, Pugh C W, Maher E R, Ratcliffe P J. The tumour suppressor protein VHL targets hypoxia-inducible factors for oxygen-dependent proteolysis. Nature 399: 271-275, 1999.
Mayers C M, Wadell J, McLean K, Venere M, Malik M, Shibata T, Driggers P H, Kino T, Guo X C, Koide H, Gorivodsky M, Grinberg A, Mukhopadhyay M, Abu-Asab M, Westphal H, Segars J H. The Rho guanine nucleotide exchange factor AKAP13 (BRX) is essential for cardiac development in mice. J Biol Chem 285: 12344-12354, 2010.
Mccartney S, Little B M, Langeberg L K, Scott J D (1995) Cloning and Characterization of a-Kinase Anchor Protein-100 (AkaplOO)—a Protein That Targets a—Kinase to the Sarcoplasmic-Reticulum. J Biol Chem 270:9327-9333.
McConnell B K, Popovic Z, Mal N, Lee K, Bautista J, Forudi F, Schwartzman R, Jin J P, Penn M, Bond M. Disruption of protein kinase A interaction with A-kinase-anchoring proteins in the heart in vivo: effects on cardiac contractility, protein kinase A phosphorylation, and troponin I proteolysis. J Biol Chem 284: 1583-1592, 2009.
McCright B, Rivers A M, Audlin S, Virshup D M (1996) The B56 family of protein phosphatase 2A (PP2A) regulatory subunits encodes differentiation-induced phosphoproteins that target PP2A to both nucleus and cytoplasm. J Biol Chem 271:22081-22089.
McKinsey T A, Kass D A. Small-molecule therapies for cardiac hypertrophy: moving beneath the cell surface. Nat Rev Drug Discov. 2007; 6:617-635.
Miano J M (2010) Role of serum response factor in the pathogenesis of disease. Lab Invest 90:1274-1284.
Michel J J, Townley I K, Dodge-Kafka K L, Zhang F, Kapiloff M S, Scott J D, “Spatial restriction of PDK1 activation cascades by anchoring to mAKAPalpha,” Mol Cell. 2005 Dec. 9; 20(5):661-72.
Michele D E, Gomez C A, Hong K E, Westfall M V, Metzger J M. Cardiac dysfunction in hypertrophic cardiomyopathy mutant tropomyosin mice is transgene-dependent, hypertrophy-independent, and improved by beta-blockade. Circ. Res. 2002; 91(3):255-262.
Monovich L, Vega R B, Meredith E, Miranda K, Rao C, Capparelli M, Lemon D D, Phan D, Koch K A, Chapo J A, Hood D B, McKinsey T A (2010) A novel kinase inhibitor establishes a predominant role for protein kinase D as a cardiac class Ila histone deacetylase kinase. FEBS Lett 584:631-637.
Morissette M R, Sah V P, Glembotski C C, Brown J H. The Rho effector, PKN, regulates ANF gene transcription in cardiomyocytes through a serum response element. Am J Physiol Heart Circ Physiol 278: H1769-H1774, 2000.
Muchir A, Bonne G, van der Kooi A J, van Meegen M, Baas F, Bolhuis P A, de Visser M, Schwartz K (2000) Identification of mutations in the gene encoding lamins A/C in autosomal dominant limb girdle muscular dystrophy with atrioventricular conduction disturbances (LGMD1B). Hum Mol Genet 9:1453-1459.
Naga Prasad S V, Barak L S, Rapacciuolo A, Caron M G, Rockman H A. Agonist-dependent recruitment of phosphoinositide 3-kinase to the membrane by beta-adrenergic receptor kinase 1. A role in receptor sequestration. J Biol Chem 276: 18953-18959, 2001.
Naga Prasad S V, Laporte S A, Chamberlain D, Caron M G, Barak L, Rockman H A. Phosphoinositide 3-kinase regulates beta₂-adrenergic receptor endocytosis by AP-2 recruitment to the receptor/beta-arrestin complex. J Cell Biol 158: 563-575, 2002.
Nakagami H, Kikuchi Y, Katsuya T, Morishita R, Akasaka H, Saitoh S, Rakugi H, Kaneda Y, Shimamoto K, Ogihara T. Gene polymorphism of myospryn (cardiomyopathy-associated 5) is associated with left ventricular wall thickness in patients with hypertension. Hypertens Res 30: 1239-1246, 2007.
Nakamura A, Rokosh D G, Paccanaro M, Yee R R, Simpson P C, Grossman W, Foster E. L V systolic performance improves with development of hypertrophy after transverse aortic constriction in mice. Am J Physiol Heart Circ Physiol. 2001; 281:H1104-1112
Nakayama K, Frew I J, Hagensen M, Skals M, Habelhah H, Bhoumik A, Kadoya T, Erdjument-Bromage H, Tempst P, Frappell P B, Bowtell D D, Ronai Z. Siah2 regulates stability of prolyl-hydroxylases, controls HIF1alpha abundance, and modulates physiological responses to hypoxia. Cell 117: 941-952, 2004.
Nauert J B, Klauck T M, Langeberg L K, Scott J D. Gravin, an autoantigen recognized by serum from myasthenia gravis patients, is a kinase scaffold protein. Curr Biol 7: 52-62., 1997.
Naya F J, Olson E (1999) MEF2: a transcriptional target for signaling pathways controlling skeletal muscle growth and differentiation. Curr Opin Cell Biol 11:683-688.
Naya F J, Wu C, Richardson J A, Overbeek P, Olson E N (1999) Transcriptional activity of MEF2 during mouse embryogenesis monitored with a MEF2-dependent transgene. Development 126:2045-2052.
Nerbonne J M, Kass R S. Molecular physiology of cardiac repolarization. Physiol Rev 85: 1205-1253, 2005.
Negro A, Dodge-Kafka K, Kapiloff M S, “Signalosomes as Therapeutic Targets,” Prog Pediatr Cardiol. 2008 April; 25(1):51-56.
Nichols C B, Rossow C F, Navedo M F, Westenbroek R E, Catterall W A, Santana L F, McKnight G S. Sympathetic stimulation of adult cardiomyocytes requires association of AKAPS with a subpopulation of L-type calcium channels. Circ Res 107: 747-756, 2010.
Newlon M G, Roy M, Morikis D, Hausken Z E, Coghlan V, Scott J D, Jennings P A (1999) The molecular basis for protein kinase A anchoring revealed by solution NMR. Nat Struct Biol 6:222-227.
Nicol R L, Frey N, Pearson G, Cobb M, Richardson J, Olson E N. Activated MEK5 induces serial assembly of sarcomeres and eccentric cardiac hypertrophy. EMBO J. 2001; 20:2757-2767.
Niggli E, Lederer W J. Voltage-independent calcium release in heart muscle. Science 250: 565-568, 1990.Papa S, Sardanelli A M, Scacco S, Petruzzella V, Technikova-Dobrova Z, Vergari R, Signorile A. The NADH: ubiquinone oxidoreductase (complex I) of the mammalian respiratory chain and the cAMP cascade. J Bioenerg Biomembr 34: 1-10, 2002.
Ohh M, Park C W, Ivan M, Hoffman M A, Kim T Y, Huang L E, Pavletich N, Chau V, Kaelin W G (2000) Ubiquitination of hypoxia-inducible factor requires direct binding to the beta-domain of the von Hippel-Lindau protein. Nat Cell Biol 2:423-427.
Oka T, Xu J, Kaiser R A, Melendez J, Hambleton M, Sargent M A, Lorts A, Brunskill E W, Dom G W, 2nd, Conway S J, Aronow B J, Robbins J, Molkentin J D. Genetic manipulation of periostin expression reveals a role in cardiac hypertrophy and ventricular remodeling. Circ. Res. 2007; 101(3):313-321.
Okumura, S., G. Takagi, J. Kawabe, G. Yang, M. C. Lee, C. Hong, J. Liu, D. E. Vatner, J. Sadoshima, S. F. Vatner and Y. Ishikawa (2003). “Disruption of type 5 adenylyl cyclase gene preserves cardiac function against pressure overload.” Proc Natl Acad Sci USA 100(17): 9986-9990.
Olson G L, Cantor C R, Braun A, Taylor S S. Designing isoform-specific peptide disruptors of protein kinase A localization. Proc Natl Acad Sci USA 100: 4072-4077, 2003.
Pare G C, Bauman A L, McHenry M, Michel J J, Dodge-Kafka K L, Kapiloff M S. The mAKAP complex participates in the induction of cardiac myocyte hypertrophy by adrenergic receptor signaling. J Cell Sci 118: 5637-5646, 2005.
Pare G C, Easlick J L, Mislow J M, McNally E M, Kapiloff M S. Nesprin-1alpha contributes to the targeting of mAKAP to the cardiac myocyte nuclear envelope. Exp Cell Res 303: 388-399, 2005.
Passariello, C. L., J. Li, K. Dodge-Kafka and M. S. Kapiloff (2015). “mAKAP-a master scaffold for cardiac remodeling.” J Cardiovasc Pharmacol 65(3): 218-225.
Passariello C L, Martinez E C, Thakur H, Cesareo M, Li J, Kapiloff M S (2016) RSK3 is required for concentric myocyte hypertrophy in an activated Rafl model for Noonan syndrome. J Mol Cell Cardiol 93:98-105.
Passariello C L, Gayanilo M, Kritzer M D, Thakur H, Cozacov Z, Rusconi F, Wieczorek D, Sanders M, Li J, Kapiloff M S (2013) p⁹⁰ ribosomal S6 kinase 3 contributes to cardiac insufficiency in alpha-tropomyosin Glul 80Gly transgenic mice. Am J Physiol Heart Circ Physiol 305:H1010-1019.
Patel H H, Hamuro L L, Chun B J, Kawaraguchi Y, Quick A, Rebolledo B, Pennypacker J, Thurston J, Rodriguez-Pinto N, Self C, Olson G, Insel P A, Giles W R, Taylor S S, Roth D M. Disruption of protein kinase A localization using a trans-activator of transcription (TAT)-conjugated A-kinase-anchoring peptide reduces cardiac function. J Biol Chem 285: 27632-27640, 2010.
Pawson C T, Scott J D. Signal integration through blending, bolstering and bifurcating of intracellular information. Nat Struct Mol Biol 17: 653-658, 2010.
Pawson T, Nash P (2003) Assembly of cell regulatory systems through protein interaction domains. Science 300:445-452.
Perino A, Ghigo A, Ferrero E, Morello F, Santulli G, Baillie G S, Damilano F, Dunlop A J, Pawson C, Walser R, Levi R, Altruda F, Silengo L, Langeberg L K, Neubauer G, S H, Lembo G, Wymann M P, Wetzker R, Houslay M D, Iaccarino G, Scott J D, Hirsch E. Integrating cardiac PIP3 and cAMP signaling through a PKA anchoring function of p110gamma. Mol Cell 42: 84-95, 2011.
Perrino C, Feliciello A, Schiattarella G G, Esposito G, Guerriero R, Zaccaro L, Del Gatto A, Saviano M, Garbi C, Carangi R, Di Lorenzo E, Donato G, Indolfi C, Avvedimento V E, Chiariello M. AKAP121 downregulation impairs protective cAMP signals, promotes mitochondrial dysfunction, and increases oxidative stress. Cardiovasc Res 88: 101-110, 2010.
Perrino C, Naga Prasad S V, Mao L, Noma T, Yan Z, Kim H S, Smithies 0, Rockman H A. Intermittent pressure overload triggers hypertrophy-independent cardiac dysfunction and vascular rarefaction. J Clin Invest. 2006; 116:1547-1560.
Peter A K, Bjerke M A, Leinwand L A (2016) Biology of the cardiac myocyte in heart disease. Mol Biol Cell 27:2149-2160.
Ponikowski, P., A. A. Voors, S. D. Anker, H. Bueno, J. G. Cleland, A. J. Coats, V. Falk, J. R. Gonzalez-Juanatey, V. P. Harjola, E. A. Jankowska, M. Jessup, C. Linde, P. Nihoyannopoulos, J. T. Parissis, B. Pieske, J. P. Riley, G. M. Rosano, L. M. Ruilope, F. Ruschitzka, F. H. Rutten, P. van der Meer and M. Authors/Task Force (2016). “2016 ESC Guidelines for the diagnosis and treatment of acute and chronic heart failure: The Task Force for the diagnosis and treatment of acute and chronic heart failure of the European Society of Cardiology (ESC) Developed with the special contribution of the Heart Failure Association (HFA) of the ESC.” Eur Heart J 37(27): 2129-2200.
Potthoff M J, Olson E N (2007) MEF2: a central regulator of diverse developmental programs. Development 134:4131-4140.
Prabhakar R, Boivin G P, Grupp I L, Hoit B, Arteaga G, Solaro J R, Wieczorek D F. A familial hypertrophic cardiomyopathy alpha-tropomyosin mutation causes severe cardiac hypertrophy and death in mice. J. Mol. Cell. Cardiol. 2001; 33(10):1815-1828.
Prasad, K. M., Y. Xu, Z. Yang, S. T. Acton and B. A. French (2011). “Robust cardiomyocyte-specific gene expression following systemic injection of AAV: in vivo gene delivery follows a Poisson distribution.” Gene Ther 18(1): 43-52.
Rababa'h A, Craft J W, Jr., Wijaya C S, Atrooz F, Fan Q, Singh S, Guillory A N, Katsonis P, Lichtarge 0, McConnell B K (2013) Protein kinase A and phosphodiesterase-4D3 binding to coding polymorphisms of cardiac muscle anchoring protein (mAKAP). J Mol Biol 425:3277-3288,
Ranganathan A, Pearson G W, Chrestensen C A, Sturgill T W, Cobb M H (2006) The MAP kinase ERK5 binds to and phosphorylates p90 RSK. Arch Biochem Biophys 449:8-16.
Reiken S, Gaburjakova M, Gaburjakova J, He Ki K L, Prieto A, Becker E, Yi Gh G H, Wang J, Burkhoff D, Marks A R (2001) beta-adrenergic receptor blockers restore cardiac calcium release channel (ryanodine receptor) structure and function in heart failure. Circulation 104:2843-2848.
Resjo S, Oknianska A, Zolnierowicz S, Manganiello V, Degerman E (1999) Phosphorylation and activation of phosphodiesterase type 38 (PDE3B) in adipocytes in response to serine/threonine phosphatase inhibitors: deactivation of PDE3B in vitro by protein phosphatase type 2A. Biochem J 341 (Pt 3):839-845.
Reynolds J G, McCalmon S A, Tomczyk T, Naya F J. Identification and mapping of protein kinase A binding sites in the costameric protein myospryn. Biochim Biophys Acta 1773: 891-902, 2007.
Richards S A, Dreisbach V C, Murphy L O, Blenis J. Characterization of regulatory events associated with membrane targeting of p90 ribosomal S6 kinase 1. Mol Cell Biol. 2001; 21:7470-7480.
Rivera V M, Miranti C K, Misr; i R P, Ginty D D, Chen R H, Blenis J, Greenberg M E (1993) A growth factor-induced kinase phosphorylates the serum response factor at a site that regulates its DNA-binding activity. Mol Cell Biol 13:6260-6273.
Rockman H A, Koch W J, Lefkowitz R J. Seven-transmembrane-spanning receptors and heart function. Nature 415: 206-212, 2002.
Rockman H A, Ross R S, Harris A N, Knowlton K U, Steinhelper M E, Field L J, Ross J Jr, Chien K R. Segregation of atrial-specific and inducible expression of an atrial natriuretic factor transgene in an in vivo murine model of cardiac hypertrophy. Proc Natl Acad Sci USA. 1991; 88:8277-8281.
Roger V L, Go A S, Lloyd-Jones D M, Adams R J, Berry J D, Brown T M, Carnethon M R, Dai S, de Simone G, Ford E S, Fox C S, Fullerton H J, Gillespie C, Greenlund K J, Hailpern S M, Heit J A, Ho P M, Howard V J, Kissela B M, Kittner S J, Lackland D T, Lichtman J H, Lisabeth L D, Makuc D M, Marcus G M, Marelli A, Matchar D B, McDermott M M, Meigs J B, Moy C S, Mozaffarian D, Mussolino M E, Nichol G, Paynter N P, Rosamond W D, Sorlie P D, Stafford R S, Turan T N, Turner M B, Wong N D, Wylie-Rosett J; American Heart Association Statistics Committee and Stroke Statistics Subcommittee. Heart disease and stroke statistics—2011 update: a report from the American Heart Association. Circulation 123: e18-e209, 2011.
Rose B A, Force T, Wang Y. Mitogen-activated protein kinase signaling in the heart: angels versus demons in a heart-breaking tale. Physiol Rev. 2010; 90:1507-1546.
Russell M A, Lund L M, Haber R, McKeegan K, Cianciola N, Bond M. The intermediate filament protein, synemin, is an AKAP in the heart. Arch Biochem Biophys 456: 204-215, 2006.
Sadoshima J, Qiu Z, Morgan J P, Izumo S. Angiotensin I I and other hypertrophic stimuli mediated by G protein-coupled receptors activate tyrosine kinase, mitogen-activated protein kinase, and 90-kD S6 kinase in cardiac myocytes. The critical role of Ca(2+)-dependent signaling. Circ. Res. 1995; 76(1):1-15.
Sapkota G P, Cummings L, Newell F S, Armstrong C, Bain J, Frodin M, Grauert M, Hoffmann M, Schnapp G, Steegmaier M, Cohen P, Alessi D R. B I-D1870 is a specific inhibitor of the p90 RSK (ribosomal S6 kinase) isoforms in vitro and in vivo. Biochem J. 2007; 401:29-38.
Schiattarella G G, Hill J A (2015) Inhibition of hypertrophy is a good therapeutic strategy in ventricular pressure overload. Circulation 131:1435-1447.
Scholten A, Poh M K, van Veen T A, van Breukelen B, Vos M A, Heck A J. Analysis of the cGMP/cAMP interactome using a chemical proteomics approach in mammalian heart tissue validates sphingosine kinase type 1-interacting protein as a genuine and highly abundant AKAP. J Proteome Res 5: 1435-1447, 2006.
Scholten A, van Veen T A, Vos M A, Heck A J. Diversity of cAMP-dependent protein kinase isoforms and their anchoring proteins in mouse ventricular tissue. J Proteome Res 6: 1705-1717, 2007.
Schulze D H, Mughal M, Lederer W J, Ruknudin A M. Sodium/calcium exchanger (NCX1) macromolecular complex. J Biol Chem 278: 28849-28855, 2003.
Scott J D, Dessauer C W, Tasken K (2013) Creating order from chaos: cellular regulation by kinase anchoring. Annu Rev Pharmacol Toxicol 53:187-210.
Scott, J. D. and T. Pawson (2009). “Cell signaling in space and time: where proteins come together and when they're apart.” Science 326(5957): 1220-1224.
Semenza G L. Hypoxia-inducible factor 1 (HIF-1) pathway. Sci STKE 2007: cm8, 2007.
Semenza G L. Regulation of oxygen homeostasis by hypoxia-inducible factor 1. Physiology 24: 97-106, 2009.
Sette C, Conti M (1996) Phosphorylation and activation of a cAMP-specific phosphodiesterase by the cAMP-dependent protein kinase. Involvement of serine 54 in the enzyme activation. J Biol Chem 271:16526-16534.
Sfichi-Duke L, Garcia-Cazarin M L, Sumandea C A, Sievert G A, Balke C W, Zhan D Y,
Morimoto S, Sumandea M P. Cardiomyopathy-causing deletion K210 in cardiac troponin T alters phosphorylation propensity of sarcomeric proteins. J Mol Cell Cardiol 48: 934-942, 2010.
Shan J, Betzenhauser M J, Kushnir A, Reiken S, Meli A C, Wronska A, Dura M, Chen B X, Marks A R. Role of chronic ryanodine receptor phosphorylation in heart failure and beta-adrenergic receptor blockade in mice. J Clin Invest 120: 4375-4387, 2010.
Shan J, Kushnir A, Betzenhauser M J, Reiken S, Li J, Lehnart S E, Lindegger N, Mongillo M, Mohler P J, Marks A R. Phosphorylation of the ryanodine receptor mediates the cardiac fight or flight response in mice. J Clin Invest 120: 4388-4398, 2010.
Sharma K, Kass D A (2014) Heart failure with preserved ejection fraction: mechanisms, clinical features, and therapies. Circ Res 115:79-96.
Shyu K G, Wang M T, Wang B W, Chang C C, Leu J G, Kuan P, Chang H. Intramyocardial injection of naked DNA encoding HIF-1alpha/VP16 hybrid to enhance angiogenesis in an acute myocardial infarction model in the rat. Cardiovasc Res 54: 576-583, 2002.
Silva, J. M., M. Z. Li, K. Chang, W. Ge, M. C. Golding, R. J. Rickles, D. Siolas, G. Hu, P. J. Paddison, M. R. Schlabach, N. Sheth, J. Bradshaw, J. Burchard, A. Kulkarni, G. Cavet, R. Sachidanandam, W. R. McCombie, M. A. Cleary, S. J. Elledge and G. J. Hannon (2005). “Second-generation shRNA libraries covering the mouse and human genomes.” Nat Genet 37(11): 1281-1288.
Singh A, Redden J M, Kapiloff M S, Dodge-Kafka K L, “The large isoforms of A-kinase anchoring protein 18 mediate the phosphorylation of inhibitor-1 by protein kinase A and the inhibition of protein phosphatase 1 activity,” Mol Pharmacol. 2011 March; 79(3):533-40.
Skroblin P, Grossmann S, Schafer G, Rosenthal W, Klussmann E. Mechanisms of protein kinase A anchoring. Int Rev Cell Mol Biol 283: 235-330, 2010.
Smith F D, Langeberg L K, Cellurale C, Pawson T, Morrison D K, Davis R J, Scott J D. AKAP-Lbc enhances cyclic AMP control of the ERK1/2 cascade. Nat Cell Biol 12: 1242-1249, 2010.
Smith J A, Poteet-Smith C E, Xu Y, Errington T M, Hecht S M, Lannigan D A. Identification of the first specific inhibitor of p90 ribosomal S6 kinase (RSK) reveals an unexpected role for RSK in cancer cell proliferation. Cancer Res. 2005; 65:1027-1034.
Spinale F G, Janicki J S, Zile M R. Membrane-associated matrix proteolysis and heart failure. Circ. Res. 2013; 112(1):195-208.
Steinberg S F, Brunton L L (2001) Compartmentation of G protein-coupled signaling pathways in cardiac myocytes. Annu Rev Pharmacol Toxicol 41:751-773.
Stelzer J E, Patel J R, Walker J W, Moss R L. Differential roles of cardiac myosin-binding protein C and cardiac troponin I in the myofibrillar force responses to protein kinase A phosphorylation. Circ Res 101: 503-511, 2007.
Sumandea C A, Garcia-Cazarin M L, Bozio C H, Sievert G A, Balke C W, Sumandea M P. Cardiac troponin T, a sarcomeric AKAP, tethers protein kinase A at the myofilaments. J Biol Chem 286: 530-541, 2011
Takeishi Y, Huang Q, Abe J, Che W, Lee J D, Kawakatsu H, Hoit B D, Berk B C, Walsh R A. Activation of mitogen-activated protein kinases and p90 ribosomal S6 kinase in failing human hearts with dilated cardiomyopathy. Cardiovasc Res. 2002; 53:131-137.
Terrenoire C, Houslay M D, Baillie G S, Kass R S. The cardiac IKs potassium channel macromolecular complex includes the phosphodies terase PDE4D3. J Biol Chem 284: 9140-9146, 2009.
Thomas G M, Rumbaugh G R, Harrar D B, Huganir R L. Ribosomal S6 kinase 2 interacts with and phosphorylates PDZ domain-containing proteins and regulates AMPA receptor transmission. Proc Natl Acad Sci USA. 2005; 102:15006-15011.
Tingley W G, Pawlikowska L, Zaroff J G, Kim T, Nguyen T, Young S G, Vranizan K, Kwok P Y, Whooley M A, Conklin B R. Gene-trapped mouse embryonic stem cell-derived cardiac myocytes and human genetics implicate AKAP10 in heart rhythm regulation. Proc Natl Acad Sci USA 104: 8461-8466, 2007.
Treisrnan R (1985) Transient accumulation of c-fos RNA following serum stimulation requires a conserved 5′ element and c-fos 3′ sequences. Cell 42:889-902.
Uys G M, Ramburan A, Loos B, Kinnear C J, Korkie L J, Mouton J, Riedemann J, Moolman-Smook J. Myomegalin is a novel A-kinase anchoring protein involved in the phosphorylation of cardiac myosin binding protein C. BMC Cell Biol 12: 18, 2011.
Valdivia H H, Kaplan J H, Ellis-Davies G C, Lederer W J (1995) Rapid adaptation of cardiac ryanodine receptors: modulation by Mg2+ and phosphorylation. Science 267:1997-2000.
Vargas M A, Tirnauer J S, Glidden N, Kapiloff M S, Dodge-Kafka K L, “Myocyte enhancer factor 2 (MEF2) tethering to muscle selective A-kinase anchoring protein (mAKAP) is necessary for myogenic differentiation,” Cell Signal. 2012 August; 24(8):1496-503.
Virshup D M (2000) Protein phosphatase 2A: a panoply of enzymes. Curr Opin Cell Biol 12:180-185.
Wang X, Tang X, Li M, Marshall J, Mao Z (2005) Regulation of neuroprotective activity of myocyte-enhancer factor 2 by cAMP-protein kinase A signaling pathway in neuronal survival. J Biol Chem 280:16705-16713.
Wang, Y., E. G. Cameron, J. Li, T. L. Stiles, M. D. Kritzer, R. Lodhavia, J. Hertz, T. Nguyen, M. S. Kapiloff and J. L. Goldberg (2015). “Muscle A-Kinase Anchoring Protein-alpha is an Injury-Specific Signaling Scaffold Required for Neurotrophic- and Cyclic Adenosine Monophosphate-Mediated Survival.” EBioMedicine 2(12): 1880-1887.
Wang, Z., H. I. Ma, J. Li, L. Sun, J. Zhang and X. Xiao (2003). “Rapid and highly efficient transduction by double-stranded adeno-associated virus vectors in vitro and in vivo.” Gene Ther 10(26): 2105-2111.
Wera S, Hemmings B A (1995) Serine/threonine protein phosphatases. Biochem J 311 (Pt 1):17-29.
Wilkins B J, De Windt L J, Bueno O F, Braz J C, Glascock B J, Kimball T F, Molkentin J D (2002) Targeted disruption of NFATc3, but not NFATc4, reveals an intrinsic defect m calcineurin-mediated cardiac hypertrophic growth. Mol Cell Biol 22:7603-7613.
Wilkins B J, Dai Y S, Bueno O F, Parsons S A, Xu J, Plank D M, Jones F, Kimball T R, Molkentin J D (2004) Calcineurin/NFAT coupling participates in pathological, but not physiological, cardiac hypertrophy. Circ Res 94:110-118.
Wong W, Goehring A S, Kapiloff M S, Langeberg L K, Scott J D, “mAKAP compartmentalizes oxygen-dependent control of HIF-1alpha,” Sci Signal. 2008 Dec. 23; 1(51).
Welch E J, Jones B W, Scott J D. Networking with AKAPs: context-dependent regulation of anchored enzymes. Mol Interv 10: 86-97, 2010. 114. Wu X, Simpson J, Hong J H, Kim K H, Thavarajah N K, Backx P H, Neel B G, Araki T. MEK-ERK pathway modulation ameliorates disease phenotypes in a mouse model of Noonan syndrome associated with the Raf1(L613V) mutation. J Clin Invest. 2011; 121:1009-1025.
Wollert K C, Taga T, Saito M, Narazaki M, Kishimoto T, Glembotski C C, Vernallis A B, Heath J K, Pennica D, Wood W I, Chien K R. Cardiotrophin-1 activates a distinct form of cardiac muscle cell hypertrophy. Assembly of sarcomeric units in series VIA gp130/leukemia inhibitory factor receptor-dependent pathways. J Biol Chem. 1996; 271:9535-9545.
Writing Group, M., D. Mozaffarian, E. J. Benjamin, A. S. Go, D. K. Arnett, M. J. Blaha, M. Cushman, S. R. Das, S. de Ferranti, J. P. Despres, H. J. Fullerton, V. J. Howard, M. D. Huffman, C. R. Isasi, M. C. Jimenez, S. E. Judd, B. M. Kissela, J. H. Lichtman, L. D. Lisabeth, S. Liu, R. H. Mackey, D. J. Magid, D. K. McGuire, E. R. Mohler, 3rd, C. S. Moy, P. Muntner, M. E. Mussolino, K. Nasir, R. W. Neumar, G. Nichol, L. Palaniappan, D. K. Pandey, M. J. Reeves, C. J. Rodriguez, W. Rosamond, P. D. Sorlie, J. Stein, A. Towfighi, T. N. Turan, S. S. Virani, D. Woo, R. W. Yeh, M. B. Turner, C. American Heart Association Statistics and S. Stroke Statistics (2016). “Heart Disease and Stroke Statistics-2016 Update: A Report From the American Heart Association.” Circulation 133(4): e38-360.
Wu H, Rothermel B, Kanatous S, Rosenberg P, Naya F J, Shelton J M, Hutcheson K A, DiMaio J M, Olson E N, Bassel-Duby R, Williams R S (2001) Activation of MEF2 by muscle activity is mediated through a calcineurin-dependent pathway. EMBO J 20:6414-6423.
Xie M, Hill J A (2013) HDAC-dependent ventricular remodeling. Trends Cardiovasc Med 23:229-235.
Xu J, Ismat F A, Wang T, Lu M M, Antonucci N, Epstein J A. Cardiomyocyte-specific loss of neurofibromin promotes cardiac hypertrophy and dysfunction. Circ Res. 2009; 105:304-311.
Yang J, Drazba J A, Ferguson D G, Bond M (1998) A-kinase anchoring protein 100 (AKAPlOO) is localized in multiple subcellular compartments in the adult rat heart. J Cell Biol 142:511-522.
Yang K C, Jay P Y, McMullen J R, Nerbonne J M. Enhanced cardiac PI3Ka signalling mitigates arrhythmogenic electrical remodelling in pathological hypertrophy and heart failure. Cardiovasc Res. 2012; 93:252-262.
Zakhary D R, Fink M A, Ruehr M L, Bond M (2000) Selectivity and regulation of A-kinase anchoring proteins in the heart. The role of autophosphorylation of the type II regulatory subunit of cAMP-dependent protein kinase. J Biol Chem 275:41389-41395.
Zhang L, Malik S, Kelley G G, Kapiloff M S, Smrcka A V, “Phospholipase Cepsilon scaffolds to muscle-specific A kinase anchoring protein (mAKAPbeta) and integrates multiple hypertrophic stimuli in cardiac myocytes,” J Biol Chem. 2011 Jul. 1; 286(26):23012-21.
Zhang, L., S. Malik, J. Pang, H. Wang, K. M. Park, D. I. Yule, B. C. Blaxall and A. V. Smrcka (2013). “Phospholipase Cepsilon hydrolyzes perinuclear phosphatidylinositol 4-phosphate to regulate cardiac hypertrophy.” Cell 153(1): 216-227.
Zhang Q, Bethmann C, Worth N F, Davies J D, Wasner C, Feuer A, Ragnauth C D, Yi Q, Mellad J A, Warren D T, Wheeler M A, Ellis J A, Skepper J N, Vorgerd M, Schlotter-Weigel B, Weissberg P L, Roberts R G, Wehnert M, Shanahan C M (2007) Nesprin-1 and -2 are involved in the pathogenesis of Emery Dreifuss muscular dystrophy and are critical for nuclear envelope integrity. Hum Mol Genet 16:2816-2833.
Zhao Y, Bjorbaek C, Moller D E. Regulation and interaction of pp90(rsk) isoforms with mitogen-activated protein kinases. J Biol Chem. 1996; 271:29773-29779.
Zhao Y, Bjorbaek C, Weremowicz S, Morton C C, Moller D E. RSK3 encodes a novel pp90rsk isoform with a unique N-terminal sequence: growth factor-stimulated kinase function and nuclear translocation. Mol Cell Biol. 1995 August; 15(8): 4353-436.

1. A method of treating or preventing heart failure with reduced ejection fraction, comprising administering to cardiac cells of a patient a composition that maintains a level of phosphorylation on serum response factor (SRF).

2. The method of claim 1, wherein SRF is phosphorylated on Serl03.

3. The method of claim 1, wherein dephosphorylation activity of protein (serine-threonine)phosphatase 2A (PP2A) is inhibited.

4. The method of claim 3, wherein anchoring of PP2A to muscle A-kinase anchoring protein (mAKAPβ) is inhibited.

5. The method of claim 4, wherein the composition comprises a fragment of mAKAPβ.

6. The method of claim 5, wherein the composition comprises an amino acid sequence having at least 90% sequence identity to a fragment of mAKAP.

7. The method of claim 5, wherein the composition comprises a fragment of amino acids 2083-2314 of mAKAP.

8. The method of claim 5, wherein the composition comprises amino acids 2132-2319 of mAKAP.

9. The method of claim 4, wherein the composition comprises a fragment of PP2A.

10. The method of claim 4, wherein said composition comprises a vector that encodes a fragment of PP2A.

11. The method of claim 4, wherein said composition comprises a vector that encodes an amino acid sequence having at least 90% sequence identity to a fragment of mAKAP.

12. The method of claim 4, wherein said composition inhibits the expression of PP2A B56δ (PPP2R5D).

13. The method of claim 10, wherein the vector encodes a fragment of amino acids 2132-2319 of mAKAP.

14. The method of claim 10, wherein the vector encodes amino acids 2132-2319 of mAKAP.

15. The method of claim 10, wherein the vector is adeno-associated virus (AAV).

16. A composition comprising a vector that encodes a molecule that inhibits the anchoring of PP2A to mAKAPβ and maintains a level of phosphorylation on serum response factor (SRF).

17. The composition of claim 16, wherein the molecule comprises a fragment of mAKAP.

18. The composition of claim 16, wherein the molecule has at least 90% sequence identity to a fragment of mAKAP.

19. The composition of claim 17, wherein the molecule is a fragment of amino acids 2132-2319 of mAKAP.

20. The composition of claim 17, wherein the molecule is amino acids 2132-2319 of mAKAP.

21. (canceled)

22. (canceled)

23. (canceled)

24. (canceled)

25. (canceled)

26. (canceled)

27. (canceled)

28. (canceled)


SEQUENCE LISTING (23 sequences)

SEQ ID NO: 1 - protein, 733 aa, Homo sapiens
MDLSMKKFAVRRFFSVYLRRKSRSKSSSLSRLEEEGVVKEIDISHHVKEGFEKADPSQFELLKVLGQGSYGKVFLVRKVKGSDAGQLYAMKVLKKATLKVRDRVRSKMERDILAEVNHPFIVKLHYAFQTEGKLYLILDFLRGGDLFTRLSKEVMFTEEDVKFYLAELALALDHLHSLGIIYRDLKPENILLDEEGHIKITDFGLSKEAIDHDKRAYSFCGTIEYMAPEVVNRRGHTQSADWWSFGVLMFEMLTGSLPFQGKDRKETMALILKAKLGMPQFLSGEAQSLLRALFKRNPCNRLGAGIDGVEEIKRHPFFVTIDWNTLYRKEIKPPFKPAVGRPEDTFHFDPEFTARTPTDSPGVPPSANAHHLFRGFSFVASSLIQEPSQQDLHKVPVHPIVQQLHGNNIHFTDGYEIKEDIGVGSYSVCKRCVHKATDTEYAVKIIDKSKRDPSEEIEILLRYGQHPNIITLKDVYDDGKFVYLVMELMRGGELLDRILRQRYFSEREASDVLCTITKTMDYLHSQGVVHRDLKPSNILYRDESGSPESIRVCDFGFAKQLRAGNGLLMTPCYTANFVAPEVLKRQGYDAACDIWSLGILLYTMLAGFTPFANGPDDTPEEILARIGSGKYALSGGNWDSISDAAKDVVSKMLHVDPHQRLTAMQVLKHPWVVNREYLSPNQLSRQDVHLVKGAMAATYFALNRTPQAPRLEPVLSSNLAQRRGMKRLTSTRL

SEQ ID NO: 2 - protein, 2314 aa, Rattus rattus
MLTMSVTLSPLRSQGPDPMATDASPMAINMTPTVEQEEGEGEEAVKAIDAEQQYGKPPPLHTAADWKIVLHLPEIETWLRMTSERVRDLTYSVQQDADSKHVDVHLVQLKDICEDISDHVEQIHALLETEFSLKLLSYSVNVIVDIHAVQLLWHQLRVSVLVLRERILQGLQDANGNYTRQTDILQAFSEETTEGRLDSLTEVDDSGQLTIKCSQDYLSLDCGITAFELSDYSPSEDLLGGLGDMTTSQAKTKSFDSWSYSEMEKEFPELIRSVGLLTVATEPVPSSCGEANEDSSQASLSDDHKGEHGEDGAPVPGQQLDSTVGMSSLDGTLANAAEHPSETAKQDSTSSPQLGAKKTQPGPCEITTPKRSIRDCFNYNEDSPTQPTLPKRGLFLKETQKNERKGSDRKGQVVDLKPELSRSTPSLVDPPDRSKLCLVLQSSYPSSPSAASQSYECLHKVGLGNLENIVRSHIKEISSSLGRLTDCHKEKLRLKKPHKTLAEVSLCRIPKQGGGSGKRSESTGSSAGPSMVSPGAPKATMRPETDSASTASGGLCHQRNRSGQLPVQSKASSSPPCSHSSESSLGSDSIKSPVPLLSKNKSQKSSPPAPCHATQNGQVVEAWYGSDEYLALPSHLKQTEVLALKLESLTKLLPQKPRGETIQDIDDWELSEMNSDSEIYPTYHIKKKHTRLGTVSPSSSSDIASSLGESIESGPLSDILSDEDLCLPLSSVKKFTDEKSERPSSSEKNESHSATRSALIQKLMHDIQHQENYEAIWERIEGFVNKLDEFIQWLNEAMETTENWTPPKAETDSLRLYLETHLSFKLNVDSHCALKEAVEEEGHQLLELVVSHKAGLKDTLRMIASQWKELQRQIKRQHSWILRALDTIKAEILATDVSVEDEEGTGSPKAEVQLCHLETQRDAVEQMSLKLYSEQYTSGSKRKEEFANMSKAHAEGSNGLLDFDSEYQELWDWLIDMESLVMDSHDLMMSEEQQQHLYKRYSVEMSIRHLKKSELLSKVEALKKGGLSLPDDILEKVDSINEKWELLGKTLREKIQDTIAGHSGSGPRDLLSPESGSLVRQLEVRIKELKRWLRDTELFIFNSCLRQEKEGTSAEKQLQYFKSLCREIKQRRRGVASILRLCQHLLDDRDTCNLNADHQPMQLIIVNLERRWEAIVMQAVQWQTRLQKKMGKESETLNVIDPGLMDLNGMSEDALEWDETDISNKLISVHEESNDLDQDPEPMLPAVKLEETHHKDSGYEEEAGDCGGSPYTSNITAPSSPHIYQVYSLHNVELHEDSHTPFLKSSPKFTGTTQPTVLTKSLSKDSSFSSTKSLPDLLGGSGLVRPYSCHSGDLSQNSGSESGIVSEGDNEMPTNSDMSLFSMVDGSPSNPETEHPDPQMGDAANVLEQKFKDNGESIKLSSVSRASVSPVGCVNGKAGDLNSVTKHTADCLGEELQGKHDVFTFYDYSYLQGSKLKLPMIMKQPQSEKAHVEDPLLGGFYFDKKSCKAKHQASESQPDAPPHERILASAPHEMGRSAYKSSDIEKTFTGIQSARQLSLLSRSSSVESLSPGGDLFGLGIFKNGSDSLQRSTSLESWLTSYKSNEDLFSCHSSGDISVSSGSVGELSKRTLDLLNRLENIQSPSEQKIKRSVSDMTLQSSSQKMPFAGQMSLDVASSINEDSPASLTELSSSDELSLCSEDIVLHKNKIPESNASFRKRLNRSVADESDVNVSMIVNVSCTSACTDDEDDSDLLSSSTLTLTEEELCLKDEDDDSSIATDDEIYEESNLMSGLDYIKNELQTWIRPKLSLTREKKRSGVTDEIKVNKDGGGNEKANPSDTLDIEALLNGSIRCLSENNGNGKTPPRTHGSGTKGENKKSTYDVSKDPHVADMENGNIESTPEREREKPQGLPEVSENLASNVKTISESELSEYEAVMDGSEDSSVARKEFCPPNDRHPPQMGPKLQHPENQSGDCKPVQNPCPGLLSEAGVGSRQDSNGLKSLPNDAPSGARKPAGCCLLEQNETEESASISSNASCCNCKPDVFHQKDDEDCSVHDFVKEIIDMASTALKSKSQPESEVAAPTSLTQIKEKVLEHSHRPIHLRKGDFYSYLSLSSHDSDCGEVTNYIDEKSSTPLPPDAVDSGLDDKEDMDCFFEACVEDEPVNEEAGLPGALPNESAIEDGAEQKSEQKTASSPVLSDKTDLVPLSGLSPQKGADDAKEGDDVSHTSQGCAESTEPTTPSGKANAEGRSRMQGVSATPEENAASAKPKIQAFSLNAKQPKGKVAMRYPSPQTLTCKEKLVNFHEDRHSNMHR

SEQ ID NO: 3 - DNA, 29 bp, synthetic construct
  misc_feature 1..29: note = chemically synthesized
  source 1..29: mol_type = other DNA
tcgacaggatgtccatattaggacatctg

SEQ ID NO: 4 - DNA, 79 bp, synthetic construct
  misc_feature 1..79: note = chemically synthesized
  source 1..79: mol_type = other DNA
gatctcgaaggtttataaagtcaatgtctgcagatgagaaagcagtggttctcttaggacttcttggggaagtgtggtc

SEQ ID NO: 5 - DNA, 13 bp, synthetic construct
  misc_feature 1..13: note = chemically synthesized
  source 1..13: mol_type = other DNA
atcgctggcagag

SEQ ID NO: 6 - DNA, 13 bp, synthetic construct
  misc_feature 1..13: note = chemically synthesized
  source 1..13: mol_type = other DNA
gagcctggatgaa

SEQ ID NO: 7 - DNA, 13 bp, synthetic construct
  misc_feature 1..13: note = chemically synthesized
  source 1..13: mol_type = other DNA
gagcctgagcgag

SEQ ID NO: 8 - protein, 2319 aa, Homo sapiens [sequence omitted]

SEQ ID NO: 9 - protein, 190 aa, Homo sapiens
MGKSSTPLPLDTTDSGLDDKEDIECFFEACVEGDSDGEEPCFSSAPPNESAVPSEAAMPLQATACSSEFSDSSLSADDADTVALSSPSSQERAEVGKEVNGLPQTSSGCAENLEFTPSKLDSEKESSGKPGESGMPEEHNAASAKSKVQDLSLKANQPTDKAALHPSPKTLTCEENLLNLHEKRHRNMHR

SEQ ID NO: 10 - DNA, 5887 bp, synthetic construct
  misc_feature 1..5887: note = chemically synthesized
  source 1..5887: mol_type = other DNA
accgagttgctctcacccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttcaattcaactactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctactcgagcctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgcagtagaaaaacagccaagctagggaggctgggaggccaagccccagataccttacatagctctgctcagcctctgtctcattaggaactccatttttaggatgcagttgtttcaggctaaaaataaatcatgcaatgaataaaaaagttagatacgacactgtagagggattcgctgatacagtctgtccgatctagagcagtctgggctttcacaagacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacctaagcttgctagcgctaccggtcgccaccatgggtaaaagcagcactccattgccactagacaccactgactcgggcttagatgacaaggaagatattgaatgcttttttgaggcctgtgttgagggtgactctgatggagaggagccttgtttctctagtgctcctccaaatgaatctgcagttcccagcgaagctgcaatgccactacaagcaacagcatgttcttctgagttcagtgatagttctctttcagctgatgatgcagatacagtggctctttcaagtccttcctctcaggaaagagctgaggttggaaaggaagtgaatggtttgccccaaacttccagtggctgtgcagaaaacttagagtttactccttcaaagcttgacagtgaaaaggaaagttccggaaaaccaggtgaatctggaatgccagaagaacataatgctgcttcagccaaatctaaagttcaagacctctccttgaaggcaaatcagccaacagacaaggccgcattgcatcccagccccaaaactttaacctgtgaagaaaatcttctaaaccttcatgaaaaacgacatagaaatatgcataggtagagtgtaatgcccccacgcatggaaatcatctcattgaaagatagcctggctgaagctcagggctagttaagtttgatccgcggccgcaatcaacctctggattacaaaatttgtgaaagattgactgatattcttaactatgttgctccttttacgctgtgtggatatgctgctttaatacctctgtatcgtgctattgcttcccgtacggctttcgttttctcctccttgtataaatcctggttgctgtctctttataaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgcggccttctgccggttctgcggcctctcccgcgtcttcgctttcggcctccgacgagtcggatctccctttgggccgcctccccgcctgtaggcctcacctgcgatctcgatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattaccactccctctctgcgcgctcgctcgctcactgaggccgggcgaccaaaggtcgcccgacgcccgggctttgcccgggcggcctcagtgagcgagcgagcgcgccagctgaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaatcagccagccggaagggccgagcgcagaagtggtcctgcaactttatccgcctccatccagtctattcattgttgccgggaagctagagtaagtagttcgccagttaatagttttcacaacgttgttgccattgctacaggcatcgtggtgtcacgctcgtcgtttggtatggcttcattcagctccggttcccaacgatcaaggcgagttacatgtcaccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcg

SEQ ID NO: 11 - protein, 190 aa, synthetic construct; identical to SEQ ID NO: 9
  REGION 1..190: note = chemically synthesized

SEQ ID NO: 12 - protein, 209 aa, synthetic construct
  REGION 1..209: note = myc-tagged rat mAKAP PBD
MEQKLISEEDLSPGMLTMSVTLSPLRSQTPLPPDAVDSGLDDKEDMDCFFEACVEDEPVNEEAGLPGALPNESAIEDGAEQKSEQKTASSPVLSDKTDLVPLSGLSPQKGADDAKEGDDVSHTSQGCAESTEPTTPSGKANAEGRSRMQGVSATPEENAASAKPKIQAFSLNAKQPKGKVAMRYPSPQTLTCKEKLVNFHEDRHSNMHR

SEQ ID NO: 13 - DNA, 4064 bp, synthetic construct
  misc_feature 1..4064: note = pscA-TnT-myc-rat mAKAP PBD plasmid
  source 1..4064: mol_type = other DNA
actcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaactcgagcctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagcagtctgggctttcacaagacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacctaagcttgctagcgctaccggtcgccaccatggagcagaaactcatctctgaagaggatctgagcccggggatgttaaccatgagcgtgacactttccccactgaggtcacagactccattgccaccggacgctgtggactctggcttagatgacaaggaagacatggactgcttctttgaagcttgtgttgaggatgagcctgtcaatgaggaagctggtctccccggtgcccttcccaatgaatcagccatcgaggatggagcagagcaaaagtcagaacaaaagacagccagctctcctgtgctcagtgacaagacagacctggtgcctctttcaggactttcccctcagaagggagctgatgatgcaaaggaaggagatgatgtgtctcacacttcccagggctgtgcagagagcacagagcctaccaccccctcaggaaaggccaatgcagaggggaggtcaagaatgcaaggtgtatcagcaacgccagaagaaaacgctgcttcggccaaaccgaaaattcaagctttctctttgaatgcaaaacagccaaaaggcaaggttgccatgaggtatcccagcccccaaactctaacctgtaaagagaagctcgtaaactttcatgaagatcgacacagtaacatgcataggtagagtgtaatgcccccacgcatggaaatcatctcattgaaagatagcctggctgaagctcagggctagttaagtttgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattaccactccctctctgcgcgctcgctcgctcactgaggccgggcgaccaaaggtcgcccgacgcccgggctttgcccgggcggcctcagtgagcgagcgagcgcgccagctgaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagt

SEQ ID NO: 14 - protein, 209 aa, synthetic construct; identical to SEQ ID NO: 12
  REGION 1..209: note = pscA-TnT-myc-rat mAKAP PBD

SEQ ID NO: 15 - DNA, 5817 bp, Homo sapiens
  source 1..5817: mol_type = unassigned DNA
gcggagaaggaggcggagggagcgattgtggccccggccgcggtggccggcgcggcctgccctttgtgaccgcagctcgcgccccacgccccgcgcccatggccgccgtgccgggctccctggccacgcgtgcccgcccgcggacctgagccccgcgcctgggatgccggggatgcgcgtcccccggccctgcggctgctccgggctgggcgcggggcgatggacctgagcatgaagaagttcgccgtgcgcaggttcttctctgtgtacctgcgcaggaagtcgcgctccaagagctccagcctgagccggctcgaggaagaaggcgtcgtgaaggagatagacatcagccatcatgtgaaggagggctttgagaaggcagatccttcccagtttgagctgctgaaggttttaggacaaggatcctatggaaaggtgttcctggtgaggaaggtgaaggggtccgacgctgggcagctctacgccatgaaggtccttaagaaagccaccctaaaagttcgggaccgagtgagatcgaagatggagagagacatcttggcagaagtgaatcaccccttcattgtgaagcttcattatgcctttcagacggaaggaaagctctacctgatcctggacttcctgcggggaggggacctcttcacccggctctccaaagaggtcatgttcacggaggaggatgtcaagttctacctggctgagctggccttggctttagaccatctccacagcctggggatcatctacagagatctgaagcctgagaacatcctcctggatgaagaggggcacattaagatcacagatttcggcctgagtaaggaggccattgaccacgacaagagagcgtactccttctgcgggacgatcgagtacatggcgcccgaggtggtgaaccggcgaggacacacgcagagtgccgactggtggtccttcggcgtgctcatgtttgagatgctcacggggtccctgccgttccaggggaaggacaggaaggagaccatggctctcatcctcaaagccaagctggggatgccgcagttcctcagtggggaggcacagagtttgctgcgagctctcttcaaacggaacccctgcaaccggctgggtgctggcattgacggagtggaggaaattaagcgccatcccttctttgtgaccatagactggaacacgctgtaccggaaggagatcaagccaccgttcaaaccagcagtgggcaggcctgaggacaccttccactttgaccccgagttcacagcgcggacgcccacagactctcctggcgtccccccgagtgcaaacgctcatcacctgtttagaggattcagctttgtggcctcaagcctgatccaggagccctcacagcaagatctgcacaaagtcccagttcacccaatcgtgcagcagttacacgggaacaacatccacttcaccgatggctacgagatcaaggaggacatcggggtgggctcctactcagtgtgcaagcgatgtgtgcataaagccacagacaccgagtatgccgtgaagatcattgataagagcaagagagacccctcggaagagattgagatcctcctgcggtacggccagcacccgaacatcatcaccctcaaggatgtctatgatgatggcaagtttgtgtacctggtaatggagctgatgcgtggtggggagctcctggaccgcatcctccggcagagatacttctcggagcgcgaagccagtgacgtcctgtgcaccatcaccaagaccatggactacctccattcccagggggttgttcatcgagacctgaagccgagtaacatcctgtacagggatgagtcggggagcccagaatccatccgagtctgcgacttcggctttgccaagcagctgcgcgcggggaacgggctgctcatgacaccctgctacacggccaatttcgtggccccggaggtcctgaagcgtcaaggctatgatgcggcgtgtgacatctggagtttggggatcctgttgtacaccatgctggcaggatttaccccttttgcaaatgggccagacgatacccctgaggagattctggcgcggatcggcagtgggaagtatgccctttctgggggaaactgggactcgatatctgacgcagctaaagacgtcgtgtccaagatgctccacgtggaccctcatcagcgcctgacggcgatgcaagtgctcaaacacccgtgggtggtcaacagagagtacctgtccccaaaccagctcagccgacaggacgtgcacctggtgaagggcgcgatggccgccacctactttgctctaaacagaacacctcaggccccgcggctggagcccgtgctgtcatccaacctggctcagcgcagaggcatgaagagactcacgtccacgcggctgtagcgggtgggaccctggccccagcgtcccctgccagcatcctcgtgggctcacagaccccggcctcggagcccgtctggcacccagagtgaccacaagtccagcagggaggcggcgcccgccctcgccgtgtccgtgttttctttttcagccccggagagggtcctgacctgggggcttctccaagcctcactgcgccagcctccccgcccgctctcttttctcccaagcgaaaccaaatgcgccccttcacctcgcgtgcccgtgcgaggccgggggcttctttcagagcccgcgggtcctctcatacatggcttctgtttctgccgagagatctgttttccaattatgaagccggtcggtttggtcagactcccgacacccacgtcccaggtacccggtgggaaagtggcagtgcgagggcgcagccattggtggttgcagggccccagagggctggggtgacctggcatcccggggctccccacgggctggatgacggggttggcactgtggcgtccaggaggagatgcctggttctgcccaaaataatccaaagagccgtttcctcctcgcccttcagtttttgcctgaggtgctgggtagcccatcctttcctctgtcccagattcaaatgaggagtaagagcccagacgagaggaaggcaggctggatctttgccttgagagctccgtgtcaccaggatggaagggggtgcctctcggaggagcctgtgtccacctccagtctcggctttccccggggggccaagcgcactgggctgccgtctgtccccagctcccgtggccacacagctatctggaggctttgcagggagtcgtgggttctcgcacctgctcagccctgtgtcggcttcctgtgtgctcacctaaagctgtggttttgctgtgttcacttcgatttttctggtctgtggagaaactgtgaattggagaaatggagctctgtggcttcccacccaaaccttctcagtccagctggaggctggagggagacacaggccccacccagcagactgaggggcagaggcacaggtgggagggcagcggagatcagcgtggacaggagcgatgcactttgtagatgctgtggctttgtgttgcgttttgtgtctctgttgcacagatctgttttttcacactgatccgtattcccctgggtgtgcacacagggcgggtgtggggcatttaggccatgctgtgctctacttcattgagtaaaatcgagtgagaggttccgggcagcaggatcgacgcccagtccagccggcagagggaacacacgggtccttcattgtcctgtaagggtgttgaagatgctccctggcggcccccaagcagactagatgggaggaggcgccgctcagcccctcaccctgcatcactgaagagcggcgcctctgcagcaagcagggcttcaggaggtgcccgctggccacagccaggttttccctaagaagatgttattttgttgggttttgttccccctccatctcgattctcgtacccaactaaaaaaaaaaaaataaagaaaaaatgtgctgcgttctgaaaaataactccttagcttggtctgattgttttcagaccttaaaatataaacttgtttcacaagctttaatccatgtggattttttttttcttagagaaccacaaaacataaaaggagcaagtcggactgaatacctgtttccatagtgcccacagggtattcctcacattttctccatagaagatgctttttcccaaggctagaacgacttccaccatgatgaatttgctttttaggtcttaattatttcacttctttttagaaacttaggaagaagtggataatcctgaggtcacacaatctgtcctcccagaaatgaacaaaagtcatcaccttttctgcttgctacacaggcaacgattcccccatcagctgcccggaccctttggcctggcttggtgtgcaggcctgtctgtttgcttaaagtcagtgggttctggtgcagggagtgagaagtgggggaagtgaaagggaaagcatccgtgagaaagcggccacggttttccctccttgtgtgcccatggggcaccagctcatggtctttttcagtcatcccagtttgtacagacttagcttctgaactctaagaatgccaaagggaccgacgagactccccatcacagcgagctctgtccttacatgtatttgatgtgcatcagcggaggagaacactggcttggccctgctccgctgagtgtctgtgaaatacctctactttccctcccatatccagaacaaaatgatacttgacatccttccacaaaagtcagcctaaagaagttatggtatcatatgttaaactaagctttcaaaaaccttagtgaaatagcaagtgactgctttcaagcagcagtcgacatgtaaatgaaggtgttcttagaattcgcattttgccagctcagcgcacctccacaacgaatgaaatgctccgtatgatttgcacaaatgacatagacctccccaaaagttaactggctctccttcctcacacagttcatcataacccaaccccccacccccgggtcatgaaaatcacagaacttataaacacattgaaccctagatctcaggcttcctgacctaccgccagtggccccttgctggccaccctatagggtcctccttccctggcagccccccatgtgggagaaatacctgattctcccaatctgcagtgggagagctttgctgaattccatcccaaagtcaaacatgggcaagaggtgaggatttcacttttaccctcaagtccgatttgtctgtgattttaaactaactgtgtatgtattgatgtttggaagattgtttgaattttaaagtgataatagtacttaatgttatccagtattgttcattaaatggtgttatcctaaagctgcacttgggatttttacctaacgctttactgattctctcaagcacatggcaaagtttgatttgcactccgttcatttctgacacgttttgctgcctcctacctttctaagcgtcatgcaaattcgagaatggagaaggacgctgccggtccctgagcggtgtggagagggcggaaggtggactccagcgcagcttgaggggctgaggacggaggctgcagcatctgtgtcgttctactgagcacgcttctctgcctcgctcctgactcagcactttgttcactggctcagcagttatgtttacacatcatttttatgttcctgctttgtaattcatgtttgagatgggtggccactgtacagatatttattacgctttccagactttctgaatagatttttttgaataaacatggttttatgaagtgtaatctttttctagcctaacaat

SEQ ID NO: 16 - DNA, 8841 bp, synthetic construct
  misc_feature 1..8841: note = rat mAKAPalpha mRNA
  source 1..8841: mol_type = other DNA
gcatcatgcagcaggtcaaacaaggcatctcctagtattgcatcctccagatgtgctgtaaacatcaaaaggagacgctgggagcaggagatgctgttttggaaagaagtaaggcttagatttctccatgttaaccatgagcgtgacactttccccactgaggtcacagggcccagatcccatggcgacggatgcttcacccatggccatcaacatgacacccactgtggagcaggaggaaggagagggagaggaagccgtgaaggccatagacgctgagcagcagtatggaaagccacctccgctccacacagcagccgactggaagattgtcctgcacttacctgagattgagacctggctccggatgacctcagagagggtccgtgacctgacctactcagtccagcaggatgcagacagcaagcatgtggatgtgcatctagttcagctgaaggacatttgtgaggatatttctgaccatgtggagcagatccatgccctccttgagacggagttttccctaaagctgctgtcctactcggtcaacgtcatcgtagacatccacgcagtacagctgctctggcaccagctccgcgtatccgtgctggtcctccgggagcgcatcctacaaggtctgcaggacgccaatggcaactacaccaggcagactgacattctgcaagcgttctctgaagaaacaacggagggccggcttgattcccttacagaagtggacgactcagggcagttaactatcaaatgttcacaggattacttgtctctggattgtggcattaccgcatttgaactctccgactacagtccaagtgaggatctgcttggtggcctgggcgacatgaccaccagccaggccaaaactaaatcttttgactcttggagctacagtgagatggagaaagagttccctgagcttatccgaagcgttgggctgcttacagtggccaccgagcctgtcccttccagctgtggagaagccaatgaggattcatctcaagcgtccctttcagatgatcacaaaggtgaacacggggaagacggtgctcccgtacctggacagcagctggactcaacggtgggaatgtcttccttagacggcacgctggcaaatgctgccgaacacccttcggagacagcaaaacaagactctacttcctccccacagcttggtgcgaagaaaacccagcctggtccttgtgaaattacgactcccaagagatccatccgcgattgctttaattataacgaggactcccccacacagcccacattacccaaaagagggctttttctaaaagaaactcaaaagaatgagcgcaaaggcagtgacaggaaggggcaggtggttgatttaaagcctgaactgagcagaagcaccccttccctggtggacccccctgacagatcgaagctctgcctagtgttgcagtcctcctaccccagcagcccttctgctgccagccagtcctatgaatgtttgcacaaggtggggctcggcaatcttgaaaacatagtcagaagtcacattaaagaaatttcttccagtctgggaaggcttactgactgccataaagagaaattgcgactgaaaaagccacacaagaccttggccgaagtgtctctgtgcagaatccctaaacagggaggcggttcaggaaagcgatctgagagcaccgggagctcagcagggcccagcatggtatcacctggagctcccaaagccacgatgagaccagaaacagattctgcgtctacagcctcaggtggcctgtgccaccagagaaatcgcagtggacaattgccagtgcagtcgaaggcctccagttcacccccttgcagtcacagcagtgaatcttctcttggctcagatagcatcaaatccccggttcctcttctttcaaaaaacaaaagccaaaaaagctccccacctgctccatgtcacgccacacagaacggtcaggtggtggaggcctggtacggctctgatgagtacctagcgctgccctctcacctgaagcagacggaggtgttagctctcaagctggagagcctaaccaagctcctaccccagaaacccagaggagagaccatccaggatattgatgactgggaactgtctgaaatgaattcagattccgaaatctatccaacataccacatcaagaaaaaacacacgagactgggcacagtgtctccaagctcatccagcgacatagcctcatctctcggggagagcattgaatccgggcccctgagtgacattctttctgacgaggacttatgtctgcccctctccagcgtgaaaaagttcactgacgagaaatcagagagaccttcatcctccgagaagaacgagagccattctgcaacaagatcagctttgattcagaaactaatgcacgatattcagcaccaagagaactatgaagccatctgggaaagaattgaggggtttgtgaacaagctggatgaattcattcagtggctaaacgaagccatggagaccaccgagaactggactcctcctaaagccgagaccgacagcctccggctgtacctggagacacacttgagttttaagttgaacgtagacagccactgtgccctcaaggaagccgtggaggaagaaggacaccaacttcttgagctcgttgtatctcacaaagcaggactgaaggacacgctgaggatgattgcgagtcaatggaaggagctgcagaggcaaatcaaacggcaacacagctggattctcagagccctggacaccatcaaagccgagatactggctactgatgtgtctgtggaggacgaggaggggacgggaagccccaaggccgaggttcagctctgccacctggaaacacagagagacgccgtggaacagatgtccctgaagctgtacagcgagcagtacaccagcgggagcaagaggaaggaagagtttgccaacatgtcgaaagcgcacgcggagggaagcaatgggcttctggactttgattcagaatatcaggagctctgggattggctgattgacatggagtccctcgtgatggacagccacgacctgatgatgtcagaggagcagcagcagcatctttacaagaggtacagtgtggaaatgtccatcaggcatctgaaaaagtcagagctactcagcaaggttgaagctttgaagaaaggtggcctttcactaccagacgatatcctggaaaaagtggattcaattaatgaaaaatgggagctgcttgggaaaaccctaagagagaagatacaggacacaatagcggggcacagtgggtcgggcccacgtgacctgctatctcctgaaagcggaagcctggtaaggcagctggaggtcaggatcaaagagctgaaaaggtggctaagagatacagagcttttcatcttcaattcctgtctgagacaagagaaggaaggaacaagcgccgagaaacagctccaatactttaagtcgctctgtcgtgagatcaagcagcggcgtcgaggagtggcctccattctgaggttgtgccagcaccttctggatgaccgggacacgtgcaacctgaacgcagatcaccagcccatgcagctgatcattgtaaacctcgagaggcggtgggaggccatcgtcatgcaagctgtccagtggcaaacacggttacaaaagaagatggggaaggaatccgagactttgaatgtgattgatcctggcttgatggacctgaatggaatgagtgaggatgccctggaatgggatgaaacagacataagtaacaaactcattagtgtgcatgaagaatcaaacgaccttgatcaagacccagagcctatgctacccgcagtgaagcttgaagagacacaccacaaggactctggttatgaagaggaggcaggtgactgtggagggtctccgtatacctcaaatatcactgcaccttccagcccacacatttaccaagtgtacagtcttcacaatgtggagctccacgaggacagccacactccatttctgaaaagcagccctaagttcacaggcacaacacagcctactgttttaactaagagcctcagcaaggactcttccttttcatctacaaaatcgttaccagaccttctagggggttccggtttggtgaggccttactcgtgtcacagtggagacttgagccagaattcaggcagtgagagtggaattgtcagcgaaggagacaacgagatgccgaccaactctgacatgagcttgttcagtatggtagacgggtccccaagtaaccctgaaacggagcatccggacccacaaatgggagatgcagccaatgtgctagagcaaaagtttaaagacaacggggaaagcattaagctttcaagtgtctctcgggcatccgtctcaccagtgggttgtgtaaatggaaaagcaggggatttaaacagtgttaccaaacacactgctgattgtttgggagaagaactacaaggaaaacatgacgtgtttacattttatgattactcgtacctccaaggctcaaaactcaaattaccaatgataatgaaacagccacagagtgaaaaggcacacgtggaggatccccttcttggtggtttttattttgataaaaagtcctgcaaagctaaacatcaggcttcagagtcacaaccagatgcgcctccccacgaaaggattctggcaagcgcgccccacgagatgggacgcagcgcatacaaaagtagcgacatagagaagacattcacgggcattcagagtgccagacagctctcccttctatctcgtagctcatctgtagagtccctttctccagggggtgatttgtttggattgggaatctttaaaaatggcagtgacagcctccagcggagcacttctttagaaagttggttgacatcctataagagcaatgaggatctctttagctgtcacagctctggggacataagtgtgagcagtggctcagttggtgagctgagtaagaggacgttagaccttctgaatcgcctggagaatatacagagcccctcggagcaaaagatcaagcggagtgtttctgacatgactctacaaagcagttcccaaaagatgcccttcgctggccagatgtcactggatgtcgcatcctccatcaatgaagactctccggcatctcttacagaactgagtagtagcgatgagctctctctttgctcggaggacattgtgttacacaaaaacaagatcccagaatccaacgcatcattcaggaagcgcctgaatcgctcagtggctgatgagagcgacgtcaatgttagcatgattgtcaatgtgtcctgcacctctgcttgcactgatgatgaagatgacagcgacctcctctccagctccactctcaccttaactgaagaagagctgtgcctcaaagatgaggatgacgactccagtattgcaacagatgatgaaatttatgaagagagcaacctgatgtctgggctggactacataaagaatgaactgcagacttggataagaccaaaactttccttgacgagagaaaagaaacggtccggtgtcactgatgaaataaaggtcaataaagatgggggaggcaatgagaaggccaatccctcggacaccctggacatcgaggcccttctcaatggctccataagatgtctttccgaaaacaacgggaatggtaagactccgcccagaactcatggctcaggaaccaaaggtgaaaataagaaaagtacgtatgacgttagtaaggatccgcacgtggctgacatggaaaatggcaatattgaaagtaccccagaaagagaaagggagaagccacaagggcttccagaggtgtcagagaaccttgcttcaaatgtgaaaacgatttctgaatctgagctcagcgagtatgaagcagtaatggatggttctgaggattcaagtgttgccagaaaggaattttgtcccccaaatgacagacatcctccacagatgggtcccaaactccagcatcccgaaaatcaaagtggcgactgcaagccagtccagaacccttgcccggggctactgtcggaagctggcgttggaagcaggcaagacagcaatggactaaaatctttgcctaacgatgcaccaagtggggctagaaaacctgccggttgctgcctgctggagcagaatgagacagaggaaagtgcttctatcagcagcaacgcttcctgttgcaactgcaagccagatgttttccatcaaaaagatgatgaagattgttcagtacatgactttgttaaggaaatcattgacatggcatcaacagccctaaaaagtaagtcacagcctgaaagtgaggtggccgcacccacatcactaacccaaattaaggagaaggtgttagagcattcgcaccggcccatacacctgagaaagggggacttttactcctacttatcactttcgtcccacgacagtgactgtggggaggtcaccaattacatagatgagaagagcagtactccattgccaccggacgctgtggactctggcttagatgacaaggaagacatggactgcttctttgaagcttgtgttgaggatgagcctgtcaatgaggaagctggtctccccggtgcccttcccaatgaatcagccatcgaggatggagcagagcaaaagtcagaacaaaagacagccagctctcctgtgctcagtgacaagacagacctggtgcctctttcaggactttcccctcagaagggagctgatgatgcaaaggaaggagatgatgtgtctcacacttcccagggctgtgcagagagcacagagcctaccaccccctcaggaaaggccaatgcagaggggaggtcaagaatgcaaggtgtatcagcaacgccagaagaaaacgctgcttcggccaaaccgaaaattcaagctttctctttgaatgcaaaacagccaaaaggcaaggttgccatgaggtatcccagcccccaaactctaacctgtaaagagaagctcgtaaactttcatgaagatcgacacagtaacatgcataggtagagtgtaatgcccccacgcatggaaatcatctcattgaaagatagcctggctgaagctcagggctagcccaatccaccctgggccggtcttgggctccatcctgttatcactgccgcctgtcacattgactttctgaagacgaaccttccttccgaatgcagtctgtccacgtgggcctctcgacctggatgtgtgcattgcttctcttaggtgatcatcctagttccacaaagctgcttgttctcccgtggattcctgtcccaagctacctctggcaaccctgtctctccagcaagacttcggttttccctccccctcctccccccccttaaagttccgcggctcaccaaattgatggtccatcaaacccactgtctggaatgatacccctcccatcagtacttgaccaatgttatgttttgctctgaaaactttcgctgtattagaccaatgtttattgaaagagatttacctaaaaagcccgcccttgatttggttgcagtatagaggagacacattgatccttctaacaaaattaagtgatgtctgaaagcgccattttaattatttctttttaaataatgatctatgcagcacttcaagaaacaactataacagtgttgtatcttataaactggtacattctactattaagtttgtttttggtttctatgcttcttgaggtggtgatgagaaaaatggttttttttttaaaacggtgtgccttgctgtattacttatagcatttattaaaaagctgctttcatggtaagattacactggtttgaaaggaggaaatagcaaggttaagatgcgtgcataatttctgtatatatgtataagctagtgcaaacactgatgtatgacagtataaaatgctttcatgtttgtgatgtccagtggtgtggaatataagccttaaacccgttcgattgcatggtaattaaaattggcataataaaaatagcttattgggggaaaggaaaattaatgatctcttctacctgtgtttaccaatttctttcatgtggttctgggaaagaaaaagaaacaaaccccatatattagcttccaaaatatccatattgcacagaaggcttaagttgcttagactacagactgggcctgaagacttcatgattttccaaatttttctgtttcactataaacatccgaaatagcaaagatttctttcccctccatcaacagcattttattctgaatgtttttatttctacttgttaatggtttaaagttgtatttggagatctcttacatgccctaatttattttaaatatttgaatgggtttggtggatggtatagaaaatttaattattattttatttaaactacagatttcaggtgtatttattttgttaaatattccatttggtcttttggtctttttatgacttgaaagtttcagcttttaatttatatcataactcctactaaagtgcctgacacacagtaggtatttcatagagtttcctgaattagagtattgggtggtttatatatatatatatatatatgagattcctgcattaaaactagaaaaagatgtgcaaagtgaaccagacacagcatattatcagatttcaaaaaggaaagagaacatagccacagaaatgacaatcattcattcagtagattagcatcttttgcctgcaagtcaccattctagattcagggagagcagctatgaccgatgcactgcctttggaggcttctgtgttagagacagagtgacctcgtgccgaattc

SEQ ID NO: 17 - DNA, 10227 bp, synthetic construct
  misc_feature 1..10227: note = human mAKAPbeta
  source 1..10227: mol_type = other DNA
gagtctgggagctgagtgactgaggatttgaaaccttgctatagttacagttcataacaagcgtctaggcagttagaccttaagtgttcaggtatggaaagaaagtcatatgttatgttttagattctgtttgtaaagctggttaactagagacagctgatgaaaaaccaaatcgacttgagttacaagatctgggctttctctgctctgctttcaacctgttggttggtggtggagtagctgacagaagcgaatggcttggctgagggacatgaagtgacagcagcctgtttaggaccacaccacattttggacctcttgctgtgcagttcaggacatttgtgaagatatttctgatcatgttgagcaaatccatgccctccttgaaacagagttctccctaaagctgctgtcttactctgtcaacgtgatagtggacatccacgcagtgcagctcctctggcaccagcttcgagtctcagtgctggttctgcgggagcgcattctgcaaggtctgcaggacgccaatggcaactacactaggcagacggacattctgcaagctttctctgaagagacaaaagagggccggcttgattctctaacagaagtggatgactcaggacaattaaccatcaaatgttctcaaaattacttgtctctggattgtggcattactgcattcgaactgtctgactacagtccaagtgaggatttgctcagtgggctaggtgacatgacctctagccaagtcaaaaccaaaccctttgactcttggagctacagtgagatggaaaaggagtttcctgagcttatccgaagtgttggtttacttacggtagctgctgactctatctctaccaatggcagtgaagcagttactgaggaggtatctcaagtatctctctcagtagacgacaaaggtggatgtgaggaagacaatgcttctgcagtcgaagagcaaccaggcttaacactgggggtgtcatcatcttcaggagaagctctgacaaatgctgctcaaccctcctctgagactgtgcagcaagaatccagttcctcctcccatcatgatgcaaagaatcagcagcctgttccttgtgaaaatgcaacccccaaacgaaccatcagagattgctttaattataacgaggactctcccacgcagcctacattgccaaaaagaggactttttcttaaagaggaaacttttaagaatgatctgaaaggcaatggtggaaagaggcaaatggttgatctaaagcctgagatgagcagaagcaccccttcgctagtagatcctcctgacagatccaaactttgcctggtattgcagtcttcttaccccaacagcccttctgctgccagccagtcttatgagtgtttacacaaggtggggaatgggaaccttgaaaacacagtcaaatttcacattaaagaaatttcttccagcctgggaaggcttaacgactgctataaagagaaatctcgacttaaaaagccacacaagacctcagaagaggtgcctccatgccgaacacctaaacgggggactggttcaggcaaacaagctaaaaatacaaagagctcagcagtgccaaatggagagctttcttatacttccaaggccatagaggggccacaaacaaattctgcttccacatcctcacttgagccttgtaatcagagaagttggaatgccaaattgcaattgcagtcagaaacatccagttcaccagcttttactcagagcagtgaatcctctgttggctcagacaacatcatgtctccggtgccacttctttcaaaacacaaaagcaaaaaaggtcaagcctcctctccaagtcacgtcactaggaatggtgaggttgtggaggcctggtatggctctgatgaatacctagcactgccctctcaccttaagcagacagaagtattggctttgaagttggaaaacctaacaaagcttctgcctcagaaacccagaggagaaaccatccagaatattgatgactgggaactgtctgaaatgaattcagattctgaaatctatccaacctatcatgtcaaaaagaagcatacaaggctaggcagggtgtctccaagctcatctagtgacatagcctcttcactaggggagagcattgaatctgggcccctgagtgacattctttctgatgaggagtccagtatgcctctcgctggcatgaaaaagtatgctgatgagaagtcagaaagagcttcatcctctgagaaaaatgagagccattctgccactaaatcagctttaattcagaaactgatgcaagatattcagcaccaagacaactatgaagccatatgggaaaaaatagaggggtttgtaaacaaactggatgaattcattcaatggttaaatgaagccatggaaactacagagaattggactccccctaaagcagagatggatgaccttaaactgtatctggagacacacttgagttttaagttgaatgtagacagtcattgtgctctcaaggaagctgtggaggaggaaggacaccaacttcttgagcttattgcatctcacaaagcaggactgaaggacatgctgcggatgattgcaagtcaatggaaggagctgcagaggcaaatcaaacggcagcacagctggattctcagggctctggataccatcaaagccgagatactggctactgatgtgtctgtggaggatgaggaagggactggaagccccaaggctgaggttcaactatgctacctggaagcacaaagagatgctgttgagcagatgtccctcaagctgtacagcgagcagtataccagcagcagcaagcgaaaggaagagtttgctgatatgtcaaaagttcattcagtgggaagcaatgggcttctggactttgattcagaatatcaggagctctgggattgcttgattgacatggagtcccttgtgatggacagccacgacctgatgatgtcagaggagcagcagcagcatctttacaagcgatacagtgtggaaatgtccatcagacacctgaaaaagacggagctgcttagtaaggttgaagctttgaagaaaggtggcgttttactaccaaatgatctccttgaaaaagtggattcaattaatgaaaaatgggaactgcttgggaaaaccctaggagagaagatccaggacacaatggcagggcacagtgggtcgagtccacgtgacctgctctctcctgaaagtggaagcctggtaaggcagctggaggtcaggatcaaagaactgaaaggatggctaagagatacagagcttttcatcttcaattcctgtctgagacaagaaaaggaaggaacaatgaatactgagaaacaactgcaatactttaagtccctctgtcgtgaaatcaagcaacgacgtcgaggagttgcctccattctgcgactatgccagcatcttttggatgaccgggagacttgcaatctgaatgcagaccaccagcccatgcagctgatcattgtaaatcttgaaagaaggtgggaagccattgtcatgcaagccgtccagtggcaaacacgtctacaaaagaagatgggaaaggaatctgagactttgaatgtgattgatcctggcttgatggacctaaatgggatgagtgaggatgccctggaatgggatgaaatggacataagtaacaagttaattagtttgaatgaggaatcaaatgaccttgatcaagaactccaacctgttatcccttccttgaagcttggagagacaagtaatgaggaccctggttatgacgaggaggctgataaccatgggggatctcagtatgcctcaaatattactgccccctctagtccacacatttaccaggtgtacagcctccacaatgttgaactctatgaggacaaccacatgccatttctgaaaaacaatccaaaggtcactggcatgacacagcctaatgttttaactaagagtctcagtaaagactcttcattttcatctaccaaatctttgccagatcttctaggtggttccaatttggtaaagccctgcgcatgtcatggaggagacatgagccagaattcaggcagtgagagtggaattgtcagtgaaggagacacagaaaccactaccaactctgaaatgtgcttgctcaatgcagtggatgggtccccaagtaaccttgaaactgaacatctggacccacaaatgggagatgcagttaacgtgttaaagcaaaaatttacagatgagggggaaagcattaagcttccaaatagctctcagtcgtccatttcaccagtgggttgtgtaaatggaaaagttggagatttaaacagtattaccaaacatacccctgactgtttgggagaagaattacaaggaaaacatgatgtgtttacattttatgattactcatacctccaaggctcaaaactcaaattaccaatgataatgaaacagtcacaaagcgaaaaagtgcatgtggaggatcccctgcttcgtggtttttattttgataaaaaatcatgcaaatctaaacatcagactacagagttacaaccagatgtacctccccatgaaaggattttggcaagtgcatctcatgaaatggatcgcatttcatataaaagtggcaatatagaaaagacattcactggcatgcagaatgccaaacagctctcccttttatctcatagttcatctattgagtccctttctccagggggtgatttatttggattgggcatctttaaaaatggcagtgacagcctccagcgaagcacttctttagaaagttggttgacttcctataaaagcaatgaagatctctttagctgtcacagctctggggatataagcgtgagcagtggctcagttggtgaactaagtaaaagaacattagatctcctgaatcgtttggagaatatccagagcccctcagagcaaaagataaaacgaagtgtttctgatatcactcttcaaagcagttcccaaaagatgtcctttactggccagatgtcattggacatagcatcttctatcaatgaagactcagcggcatctctaacagaacttagcagcagtgacgagctctctctttgctcagaggatattgtgttacacaagaacaagatcccggaatcgaatgcatcgttcaggaagcgtctgactcgttcagtggctgatgaaagcgatgtcaatgtcagcatgattgttaatgtctcttgcacctctgcttgcactgatgatgaagatgacagcgacctgctctccagctctacccttaccttgactgaagaagagctgtgcatcaaagatgaggatgacgactccagtattgcaacagatgatgaaatttatgaagactgcaccttgatgtcagggctagactacataaagaatgaattacagacctggattaggccaaaattgtctttgacaagagataagaaaaggtgcaatgtcagtgatgagatgaagggcagtaaagatataagtagcagtgagatgaccaatccctctgatactctgaatattgagacccttctaaatggctctgtaaaacgtgtctctgaaaataatggaaatggtaagaattcatctcatacccatgagttagggacaaagcgtgaaaataagaaaactattttcaaagttaataaagatccatatgtggctgacatggaaaatggcaatattgaaggtattccagaaaggcaaaagggcaaaccgaatgtgacttcaaaggtatcagaaaatcttggttcacatgggaaagagatttcagagagtgagcattgtaagtgtaaagcacttatggatagtttagatgattcaaatactgctggcaaggaatttgtttcccaagatgttagacatcttccaaagaaatgtccaaatcaccaccattttgaaaatcaaagcactgcctctactcccactgagaagtctttctcagaactggctttagaaaccaggtttaacaacagacaagactctgatgcactgaaatcatctgatgatgcaccgagtatggctggaaaatctgctggttgttgcctagcacttgaacaaaacggaacagaggaaaatgcttctatcagcaacatttcctgttgcaactgtgagccagatgttttccatcaaaaagatgccgaagattgttcagtacacaactttgttaaggaaatcattgacatggcttcgacagccctaaaaagtaaatctcaacctgaaaacgaggtggctgctcctacttcattaactcaaatcaaggagaaagtgttggagcattctcaccggcccatccagctgagaaaaggggacttttattcgtacttatctctctcatctcatgacagtgattgtggggaggtcaccaattacatagaagagaaaagcagcactccattgccactagacaccactgactcgggcttagatgacaaggaagatattgaatgcttttttgaggcctgtgttgagggtgactctgatggagaggagccttgtttctctagtgctcctccaaatgaatctgcagttcccagcgaagctgcaatgccactacaagcaacagcatgttcttctgagttcagtgatagttctctttcagctgatgatgcagatacagtggctctttcaagtccttcctctcaggaaagagctgaggttggaaaggaagtgaatggtttgccccaaacttccagtggctgtgcagaaaacttagagtttactccttcaaagcttgacagtgaaaaggaaagttccggaaaaccaggtgaatctggaatgccagaagaacataatgctgcttcagccaaatctaaagttcaagacctctccttgaaggcaaatcagccaacagacaaggccgcattgcatcccagccccaaaactttaacctgtgaagaaaatcttctaaaccttcatgaaaaacgacatagaaatatgcataggtagaatgtaccccctccccaagcatgaaaatcatctcactgaaagatacgcctggctgcaactcaggggtggcctcatcctcccgccctgggctggcctctggttccatcacgtttgtcactgccgtttattacattgacttctcccaagatgaatcttccttccaaatgtgttttctccacacaagccttgtgatctgaatgtgtgcgctggttctctttaggtgatcgtctttgaagttcagcaaagctgcttgttctcccatggattcctgtcccaagctacctctaccaaccctctctctccagctagacttttctctttgcctcctcccttcccttccactctttaaagttctgcagttcaccaactggtagtccattaaattctcctgtctagaatgacccccccaccagtacttgaccaatttcatgtatcaatctggattttttttttaacggtataatgactgtgcttattgaaagagttttacctaaaaagccaacatttgaattggttgcagcatagagaagaaacactggtccttctttcaaaattaagcaactattaaaagcgccattttatttatttcatttaaaaaataatctatgcagcatttcaagaaacaaccatatggtgttgtatattataaactggtgacattctactattgaattatgtacaacattttcattttttatgcttcttgaggtggtaatgagaaaaaagttttttaaaaaagtgtgccttgctgtatttcttataccatttattaaaaagctgctttcacggtaaaattatgttggtttgaaaggaggaaatagcaaggttaagatgtgtgaataatttctgtatatatgtataaccaagtacaaacattgatgtataatgacagtataaaatgctttcatgtttgtgatgtctagtgatgtggaaaatataagccttaaatccattagattgcatggtaattaaaattggcataataaacacagattattgggggaaaaggaaaattagtgatctcttctactatgttctttaccaaattgttgcatctggttctgaaaaagtatagcatgtagcagcttccaaacatattcatattgcttaagaggcttaacattacctaaactagagactagacgtaaagccttcagttttcaaaatctttctggtcactataaagatcttggaacagcaaatgattaaatgtcagttcccctaaaccaataaacatttatactagattttttatttccacttatcattaatgatttaatgttggatttcaggtaccttgtatgtcttaatttattttaaatatttattttgaatgagtttgatagaaagctagtagaaaagtacagaaaatttgactattatttatagatttcaggtatatttatatgtgtaaaagaaattgacaaagaaatatttcatctggcctttactgactcctgttaaatgcagttttaaatttatatcgtaacacctacttaagtgcctgacacagtaggtattcaataaaaatttactgaattaaaggattaaattaggtgacatggtgacatctatccctttattttgacactaaaacatggacacaactagaaagaggtacaatgcaatataaagtcacaatagataatatatatcaaatttctaaaaggtaaagaatgttgtgggttcatgcagtcacaggaatgacaatcattcaacagatagttcagaaacactttttatctgcaaggcactattctagatccagaagatgcaatgttgaacaaacagacaaagccctgccctcagaaggctgtcctgcattaggaacaagtgaacacgcaaatgacatgaagtatttgttgcagagctgaggaacagagcaaatgtagtgatagaagcgcaatgagagaagcagcagtgggtacaaggaggaagaaaaagggcttgcagagagtggaaagttagtggaatattcatgaaacttcattgcaggggtaatagaagaaaaagtaaattgggaggacttaatggaaggtcttttaaaaagttaacttggagcttctgtatgtaaaatgctaggtaataaggacactttgtacaggctgttttgcacctgattttatttatcattagtgccacgccaagatcatttagacgatgcttatctgtaattctaccactttaataactatttgtatttttatgccccttctgatcttttccatatgtatttctaaatggataaattattctaggcttcttaataggtagtaatttgttcaaaagcggttttagccagacatctagttgcagtgttcaagaggattatgggggaaagagattagagataattgtctagttagggggcagctggagaaaataagctaagtttgcaataacagagtacacaagtatagtggcccaggatgtagtgaaagaacaaatcctagagtctttgaaatttctaagggcattctagacctctgttgggatatggtattattttacatactgacacaacctaaattttctttgggtagtaactaatgtcaagtctacatcgactggtaaaacattcaaagaacaaactgacaatgatgttctacctacttgttacatgctcatggaagaccgtgcagtattgaaagtatttgttaattatctgcttagtattaacactaaatttgtagaatgactttcaggtttgttgaacaatgccttttcaggttggaagaagaaaaatagcctcaatctcccaccccatgtaggcactacctccccaattacccttagaaaatgatcacaccaactctgcctacacacttccagtgatagtggctcattgtctgttaaggcaaactgttccactgttgggcatatctctttgttagaaagttctttcttaggttgctaaaatctgcctagtaccccgctaccctgttctgtcttatggagcagcccagattatctttactccctctttctcatggcaaccctgaagataatcaaggccagttactcatcatctcccaaccactgtttcctcaactgcccttcatatgtcatggttttcagatccattccaacctgactgaatgttaacagacagaattcttcacattaaggaactgtcttcatcatcatacatgtagaaaagaatctgaacatttaagtgcgaagttttctctagaaatatattcaagatatgtttattctattattgtaaatttcaaacaataaataaataagaatcc

SEQ ID NO: 18 - protein, 2077 aa, synthetic construct
  REGION 1..2077: note = human mAKAPbeta
MTSSQVKTKPFDSWSYSEMEKEFPELIRSVGLLTVAADSISTNGSEAVTEEVSQVSLSVDDKGGCEEDNASAVEEQPGLTLGVSSSSGEALTNAAQPSSETVQQESSSSSHHDAKNQQPVPCENATPKRTIRDCFNYNEDSPTQPTLPKRGLFLKEETFKNDLKGNGGKRQMVDLKPEMSRSTPSLVDPPDRSKLCLVLQSSYPNSPSAASQSYECLHKVGNGNLENTVKFHIKEISSSLGRLNDCYKEKSRLKKPHKTSEEVPPCRTPKRGTGSGKQAKNTKSSAVPNGELSYTSKAIEGPQTNSASTSSLEPCNQRSWNAKLQLQSETSSSPAFTQSSESSVGSDNIMSPVPLLSKHKSKKGQASSPSHVTRNGEVVEAWYGSDEYLALPSHLKQTEVLALKLENLTKLLPQKPRGETIQNIDDWELSEMNSDSEIYPTYHVKKKHTRLGRVSPSSSSDIASSLGESIESGPLSDILSDEESSMPLAGMKKYADEKSERASSSEKNESHSATKSALIQKLMQDIQHQDNYEAIWEKIEGFVNKLDEFIQWLNEAMETTENWTPPKAEMDDLKLYLETHLSFKLNVDSHCALKEAVEEEGHQLLELIASHKAGLKDMLRMIASQWKELQRQIKRQHSWILRALDTIKAEILATDVSVEDEEGTGSPKAEVQLCYLEAQRDAVEQMSLKLYSEQYTSSSKRKEEFADMSKVHSVGSNGLLDFDSEYQELWDCLIDMESLVMDSHDLMMSEEQQQHLYKRYSVEMSIRHLKKTELLSKVEALKKGGVLLPNDLLEKVDSINEKWELLGKTLGEKIQDTMAGHSGSSPRDLLSPESGSLVRQLEVRIKELKGWLRDTELFIFNSCLRQEKEGTMNTEKQLQYFKSLCREIKQRRRGVASILRLCQHLLDDRETCNLNADHQPMQLIIVNLERRWEAIVMQAVQWQTRLQKKMGKESETLNVIDPGLMDLNGMSEDALEWDEMDISNKLISLNEESNDLDQELQPVIPSLKLGETSNEDPGYDEEADNHGGSQYASNITAPSSPHIYQVYSLHNVELYEDNHMPFLKNNPKVTGMTQPNVLTKSLSKDSSFSSTKSLPDLLGGSNLVKPCACHGGDMSQNSGSESGIVSEGDTETTTNSEMCLLNAVDGSPSNLETEHLDPQMGDAVNVLKQKFTDEGESIKLPNSSQSSISPVGCVNGKVGDLNSITKHTPDCLGEELQGKHDVFTFYDYSYLQGSKLKLPMIMKQSQSEKVHVEDPLLRGFYFDKKSCKSKHQTTELQPDVPPHERILASASHEMDRISYKSGNIEKTFTGMQNAKQLSLLSHSSSIESLSPGGDLFGLGIFKNGSDSLQRSTSLESWLTSYKSNEDLFSCHSSGDISVSSGSVGELSKRTLDLLNRLENIQSPSEQKIKRSVSDITLQSSSQKMSFTGQMSLDIASSINEDSAASLTELSSSDELSLCSEDIVLHKNKIPESNASFRKRLTRSVADESDVNVSMIVNVSCTSACTDDEDDSDLLSSSTLTLTEEELCIKDEDDDSSIATDDEIYEDCTLMSGLDYIKNELQTWIRPKLSLTRDKKRCNVSDEMKGSKDISSSEMTNPSDTLNIETLLNGSVKRVSENNGNGKNSSHTHELGTKRENKKTIFKVNKDPYVADMENGNIEGIPERQKGKPNVTSKVSENLGSHGKEISESEHCKCKALMDSLDDSNTAGKEFVSQDVRHLPKKCPNHHHFENQSTASTPTEKSFSELALETRFNNRQDSDALKSSDDAPSMAGKSAGCCLALEQNGTEENASISNISCCNCEPDVFHQKDAEDCSVHNFVKEIIDMASTALKSKSQPENEVAAPTSLTQIKEKVLEHSHRPIQLRKGDFYSYLSLSSHDSDCGEVTNYIEEKSSTPLPLDTTDSGLDDKEDIECFFEACVEGDSDGEEPCFSSAPPNESAVPSEAAMPLQATACSSEFSDSSLSADDADTVALSSPSSQERAEVGKEVNGLPQTSSGCAENLEFTPSKLDSEKESSGKPGESGMPEEHNAASAKSKVQDLSLKANQPTDKAALHPSPKTLTCEENLLNLHEKRHRNMHR

SEQ ID NO: 19 - DNA, 10343 bp, synthetic construct
  misc_feature 1..10343: note = human mAKAPalpha
  source 1..10343: mol_type = other DNA
catcatgcagcaggtcaaacaaggcatctcctagtattgcatcctacagatgtgctgtaaacatcaaaagaagacggtgggatcaggagatgctgttttggaaagaagtgaggtttagacttctccatgttaaccatgagcgtgacactttcccccctgaggtcacaggacctggatcccatggctactgatgcttcacccatggccatcaacatgacacccactgtggagcagggtgagggagaagaggcaatgaaggacatggactctgaccagcagtatgaaaagccacccccactacacacaggggctgactggaagattgtcctccacttacctgaaattgagacctggctccggatgacctcagagagggtccgagacctaacctattcagtccagcaggattcggacagcaagcatgtggatgtacatctagttcaactaaaggacatttgtgaagatatttctgatcatgttgagcaaatccatgccctccttgaaacagagttctccctaaagctgctgtcttactctgtcaacgtgatagtggacatccacgcagtgcagctcctctggcaccagcttcgagtctcagtgctggttctgcgggagcgcattctgcaaggtctgcaggacgccaatggcaactacactaggcagacggacattctgcaagctttctctgaagagacaaaagagggccggcttgattctctaacagaagtggatgactcaggacaattaaccatcaaatgttctcaaaattacttgtctctggattgtggcattactgcattcgaactgtctgactacagtccaagtgaggatttgctcagtgggctaggtgacatgacctctagccaagtcaaaaccaaaccctttgactcttggagctacagtgagatggaaaaggagtttcctgagcttatccgaagtgttggtttacttacggtagctgctgactctatctctaccaatggcagtgaagcagttactgaggaggtatctcaagtatctctctcagtagacgacaaaggtggatgtgaggaagacaatgcttctgcagtcgaagagcaaccaggcttaacactgggggtgtcatcatcttcaggagaagctctgacaaatgctgctcaaccctcctctgagactgtgcagcaagaatccagttcctcctcccatcatgatgcaaagaatcagcagcctgttccttgtgaaaatgcaacccccaaacgaaccatcagagattgctttaattataacgaggactctcccacgcagcctacattgccaaaaagaggactttttcttaaagaggaaacttttaagaatgatctgaaaggcaatggtggaaagaggcaaatggttgatctaaagcctgagatgagcagaagcaccccttcgctagtagatcctcctgacagatccaaactttgcctggtattgcagtcttcttaccccaacagcccttctgctgccagccagtcttatgagtgtttacacaaggtggggaatgggaaccttgaaaacacagtcaaatttcacattaaagaaatttcttccagcctgggaaggcttaacgactgctataaagagaaatctcgacttaaaaagccacacaagacctcagaagaggtgcctccatgccgaacacctaaacgggggactggttcaggcaaacaagctaaaaatacaaagagctcagcagtgccaaatggagagctttcttatacttccaaggccatagaggggccacaaacaaattctgcttccacatcctcacttgagccttgtaatcagagaagttggaatgccaaattgcaattgcagtcagaaacatccagttcaccagcttttactcagagcagtgaatcctctgttggctcagacaacatcatgtctccggtgccacttctttcaaaacacaaaagcaaaaaaggtcaagcctcctctccaagtcacgtcactaggaatggtgaggttgtggaggcctggtatggctctgatgaatacctagcactgccctctcaccttaagcagacagaagtattggctttgaagttggaaaacctaacaaagcttctgcctcagaaacccagaggagaaaccatccagaatattgatgactgggaactgtctgaaatgaattcagattctgaaatctatccaacctatcatgtcaaaaagaagcatacaaggctaggcagggtgtctccaagctcatctagtgacatagcctcttcactaggggagagcattgaatctgggcccctgagtgacattctttctgatgaggagtccagtatgcctctcgctggcatgaaaaagtatgctgatgagaagtcagaaagagcttcatcctctgagaaaaatgagagccattctgccactaaatcagctttaattcagaaactgatgcaagatattcagcaccaagacaactatgaagccatatgggaaaaaatagaggggtttgtaaacaaactggatgaattcattcaatggttaaatgaagccatggaaactacagagaattggactccccctaaagcagagatggatgaccttaaactgtatctggagacacacttgagttttaagttgaatgtagacagtcattgtgctctcaaggaagctgtggaggaggaaggacaccaacttcttgagcttattgcatctcacaaagcaggactgaaggacatgctgcggatgattgcaagtcaatggaaggagctgcagaggcaaatcaaacggcagcacagctggattctcagggctctggataccatcaaagccgagatactggctactgatgtgtctgtggaggatgaggaagggactggaagccccaaggctgaggttcaactatgctacctggaagcacaaagagatgctgttgagcagatgtccctcaagctgtacagcgagcagtataccagcagcagcaagcgaaaggaagagtttgctgatatgtcaaaagttcattcagtgggaagcaatgggcttctggactttgattcagaatatcaggagctctgggattgcttgattgacatggagtcccttgtgatggacagccacgacctgatgatgtcagaggagcagcagcagcatctttacaagcgatacagtgtggaaatgtccatcagacacctgaaaaagacggagctgcttagtaaggttgaagctttgaagaaaggtggcgttttactaccaaatgatctccttgaaaaagtggattcaattaatgaaaaatgggaactgcttgggaaaaccctaggagagaagatccaggacacaatggcagggcacagtgggtcgagtccacgtgacctgctctctcctgaaagtggaagcctggtaaggcagctggaggtcaggatcaaagaactgaaaggatggctaagagatacagagcttttcatcttcaattcctgtctgagacaagaaaaggaaggaacaatgaatactgagaaacaactgcaatactttaagtccctctgtcgtgaaatcaagcaacgacgtcgaggagttgcctccattctgcgactatgccagcatcttttggatgaccgggagacttgcaatctgaatgcagaccaccagcccatgcagctgatcattgtaaatcttgaaagaaggtgggaagccattgtcatgcaagccgtccagtggcaaacacgtctacaaaagaagatgggaaaggaatctgagactttgaatgtgattgatcctggcttgatggacctaaatgggatgagtgaggatgccctggaatgggatgaaatggacataagtaacaagttaattagtttgaatgaggaatcaaatgaccttgatcaagaactccaacctgttatcccttccttgaagcttggagagacaagtaatgaggaccctggttatgacgaggaggctgataaccatgggggatctcagtatgcctcaaatattactgccccctctagtccacacatttaccaggtgtacagcctccacaatgttgaactctatgaggacaaccacatgccatttctgaaaaacaatccaaaggtcactggcatgacacagcctaatgttttaactaagagtctcagtaaagactcttcattttcatctaccaaatctttgccagatcttctaggtggttccaatttggtaaagccctgcgcatgtcatggaggagacatgagccagaattcaggcagtgagagtggaattgtcagtgaaggagacacagaaaccactaccaactctgaaatgtgcttgctcaatgcagtggatgggtccccaagtaaccttgaaactgaacatctggacccacaaatgggagatgcagttaacgtgttaaagcaaaaatttacagatgagggggaaagcattaagcttccaaatagctctcagtcgtccatttcaccagtgggttgtgtaaatggaaaagttggagatttaaacagtattaccaaacatacccctgactgtttgggagaagaattacaaggaaaacatgatgtgtttacattttatgattactcatacctccaaggctcaaaactcaaattaccaatgataatgaaacagtcacaaagcgaaaaagtgcatgtggaggatcccctgcttcgtggtttttattttgataaaaaatcatgcaaatctaaacatcagactacagagttacaaccagatgtacctccccatgaaaggattttggcaagtgcatctcatgaaatggatcgcatttcatataaaagtggcaatatagaaaagacattcactggcatgcagaatgccaaacagctctcccttttatctcatagttcatctattgagtccctttctccagggggtgatttatttggattgggcatctttaaaaatggcagtgacagcctccagcgaagcacttctttagaaagttggttgacttcctataaaagcaatgaagatctctttagctgtcacagctctggggatataagcgtgagcagtggctcagttggtgaactaagtaaaagaacattagatctcctgaatcgtttggagaatatccagagcccctcagagcaaaagataaaacgaagtgtttctgatatcactcttcaaagcagttcccaaaagatgtcctttactggccagatgtcattggacatagcatcttctatcaatgaagactcagcggcatctctaacagaacttagcagcagtgacgagctctctctttgctcagaggatattgtgttacacaagaacaagatcccggaatcgaatgcatcgttcaggaagcgtctgactcgttcagtggctgatgaaagcgatgtcaatgtcagcatgattgttaatgtctcttgcacctctgcttgcactgatgatgaagatgacagcgacctgctctccagctctacccttaccttgactgaagaagagctgtgcatcaaagatgaggatgacgactccagtattgcaacagatgatgaaatttatgaagactgcaccttgatgtcagggctagactacataaagaatgaattacagacctggattaggccaaaattgtctttgacaagagataagaaaaggtgcaatgtcagtgatgagatgaagggcagtaaagatataagtagcagtgagatgaccaatccctctgatactctgaatattgagacccttctaaatggctctgtaaaacgtgtctctgaaaataatggaaatggtaagaattcatctcatacccatgagttagggacaaagcgtgaaaataagaaaactattttcaaagttaataaagatccatatgtggctgacatggaaaatggcaatattgaaggtattccagaaaggcaaaagggcaaaccgaatgtgacttcaaaggtatcagaaaatcttggttcacatgggaaagagatttcagagagtgagcattgtaagtgtaaagcacttatggatagtttagatgattcaaatactgctggcaaggaatttgtttcccaagatgttagacatcttccaaagaaatgtccaaatcaccaccattttgaaaatcaaagcactgcctctactcccactgagaagtctttctcagaactggctttagaaaccaggtttaacaacagacaagactctgatgcactgaaatcatctgatgatgcaccgagtatggctggaaaatctgctggttgttgcctagcacttgaacaaaacggaacagaggaaaatgcttctatcagcaacatttcctgttgcaactgtgagccagatgttttccatcaaaaagatgccgaagattgttcagtacacaactttgttaaggaaatcattgacatggcttcgacagccctaaaaagtaaatctcaacctgaaaacgaggtggctgctcctacttcattaactcaaatcaaggagaaagtgttggagcattctcaccggcccatccagctgagaaaaggggacttttattcgtacttatctctctcatctcatgacagtgattgtggggaggtcaccaattacatagaagagaaaagcagcactccattgccactagacaccactgactcgggcttagatgacaaggaagatattgaatgcttttttgaggcctgtgttgagggtgactctgatggagaggagccttgtttctctagtgctcctccaaatgaatctgcagttcccagcgaagctgcaatgccactacaagcaacagcatgttcttctgagttcagtgatagttctctttcagctgatgatgcagatacagtggctctttcaagtccttcctctcaggaaagagctgaggttggaaaggaagtgaatggtttgccccaaacttccagtggctgtgcagaaaacttagagtttactccttcaaagcttgacagtgaaaaggaaagttccggaaaaccaggtgaatctggaatgccagaagaacataatgctgcttcagccaaatctaaagttcaagacctctccttgaaggcaaatcagccaacagacaaggccgcattgcatcccagccccaaaactttaacctgtgaagaaaatcttctaaaccttcatgaaaaacgacatagaaatatgcataggtagaatgtaccccctccccaagcatgaaaatcatctcactgaaagatacgcctggctgcaactcaggggtggcctcatcctcccgccctgggctggcctctggttccatcacgtttgtcactgccgtttattacattgacttctcccaagatgaatcttccttccaaatgtgttttctccacacaagccttgtgatctgaatgtgtgcgctggttctctttaggtgatcgtctttgaagttcagcaaagctgcttgttctcccatggattcctgtcccaagctacctctaccaaccctctctctccagctagacttttctctttgcctcctcccttcccttccactctttaaagttctgcagttcaccaactggtagtccattaaattctcctgtctagaatgacccccccaccagtacttgaccaatttcatgtatcaatctggattttttttttaacggtataatgactgtgcttattgaaagagttttacctaaaaagccaacatttgaattggttgcagcatagagaagaaacactggtccttctttcaaaattaagcaactattaaaagcgccattttatttatttcatttaaaaaataatctatgcagcatttcaagaaacaaccatatggtgttgtatattataaactggtgacattctactattgaattatgtacaacattttcattttttatgcttcttgaggtggtaatgagaaaaaagttttttaaaaaagtgtgccttgctgtatttcttataccatttattaaaaagctgctttcacggtaaaattatgttggtttgaaaggaggaaatagcaaggttaagatgtgtgaataatttctgtatatatgtataaccaagtacaaacattgatgtataatgacagtataaaatgctttcatgtttgtgatgtctagtgatgtggaaaatataagccttaaatccattagattgcatggtaattaaaattggcataataaacacagattattgggggaaaaggaaaattagtgatctcttctactatgttctttaccaaattgttgcatctggttctgaaaaagtatagcatgtagcagcttccaaacatattcatattgcttaagaggcttaacattacctaaactagagactagacgtaaagccttcagttttcaaaatctttctggtcactataaagatcttggaacagcaaatgattaaatgtcagttcccctaaaccaataaacatttatactagattttttatttccacttatcattaatgatttaatgttggatttcaggtaccttgtatgtcttaatttattttaaatatttattttgaatgagtttgatagaaagctagtagaaaagtacagaaaatttgactattatttatagatttcaggtatatttatatgtgtaaaagaaattgacaaagaaatatttcatctggcctttactgactcctgttaaatgcagttttaaatttatatcgtaacacctacttaagtgcctgacacagtaggtattcaataaaaatttactgaattaaaggattaaattaggtgacatggtgacatctatccctttattttgacactaaaacatggacacaactagaaagaggtacaatgcaatataaagtcacaatagataatatatatcaaatttctaaaaggtaaagaatgttgtgggttcatgcagtcacaggaatgacaatcattcaacagatagttcagaaacactttttatctgcaaggcactattctagatccagaagatgcaatgttgaacaaacagacaaagccctgccctcagaaggctgtcctgcattaggaacaagtgaacacgcaaatgacatgaagtatttgttgcagagctgaggaacagagcaaatgtagtgatagaagcgcaatgagagaagcagcagtgggtacaaggaggaagaaaaagggcttgcagagagtggaaagttagtggaatattcatgaaacttcattgcaggggtaatagaagaaaaagtaaattgggaggacttaatggaaggtcttttaaaaagttaacttggagcttctgtatgtaaaatgctaggtaataaggacactttgtacaggctgttttgcacctgattttatttatcattagtgccacgccaagatcatttagacgatgcttatctgtaattctaccactttaataactatttgtatttttatgccccttctgatcttttccatatgtatttctaaatggataaattattctaggcttcttaataggtagtaatttgttcaaaagcggttttagccagacatctagttgcagtgttcaagaggattatgggggaaagagattagagataattgtctagttagggggcagctggagaaaataagctaagtttgcaataacagagtacacaagtatagtggcccaggatgtagtgaaagaacaaatcctagagtctttgaaatttctaagggcattctagacctctgttgggatatggtattattttacatactgacacaacctaaattttctttgggtagtaactaatgtcaagtctacatcgactggtaaaacattcaaagaacaaactgacaatgatgttctacctacttgttacatgctcatggaagaccgtgcagtattgaaagtatttgttaattatctgcttagtattaacactaaatttgtagaatgactttcaggtttgttgaacaatgccttttcaggttggaagaagaaaaatagcctcaatctcccaccccatgtaggcactacctccccaattacccttagaaaatgatcacaccaactctgcctacacacttccagtgatagtggctcattgtctgttaaggcaaactgttccactgttgggcatatctctttgttagaaagttctttcttaggttgctaaaatctgcctagtaccccgctaccctgttctgtcttatggagcagcccagattatctttactccctctttctcatggcaaccctgaagataatcaaggccagttactcatcatctcccaaccactgtttcctcaactgcccttcatatgtcatggttttcagatccattccaacctgactgaatgttaacagacagaattcttcacattaaggaactgtcttcatcatcatacatgtagaaaagaatctgaacatttaagtgcgaagttttctctagaaatatattcaagatatgtttattctattattgtaaatttcaaacaataaataaataagaatcc

SEQ ID NO: 20 - protein, 2319 aa, synthetic construct [sequence omitted]

SEQ ID NO: 21 - protein, 10 aa, synthetic construct
  REGION 1..10: note = Myc-tag
EQKLISEEDL

SEQ ID NO: 22 - protein, 189 aa, synthetic construct
  REGION 1..189: note = mAKAP PBD
  VARIANT 1: note = MISC_FEATURE - Xaa is Lys or Arg
  VARIANT 3: note = MISC_FEATURE - Xaa can be any naturally occurring amino acid
  VARIANT 8: note = MISC_FEATURE - Xaa can be any naturally occurring amino acid
  VARIANT 10..11: note = MISC_FEATURE - Xaa can be any naturally occurring amino acid
  VARIANT 21: note = MISC_FEATURE - Xaa is Ile or Met
  VARIANT 22: note = MISC_FEATURE - Xaa is Glu or Asp
  VARIANT 31: note = MISC_FEATURE - Xaa can be any naturally occurring amino acid
  VARIANT 32: note = MISC_FEATURE - Xaa is Asp or Glu
  VARIANT 33..35: note = MISC_FEATURE - Xaa can be any naturally occurring amino acid
  VARIANT 38..42: note = MISC_FEATURE - Xaa can be any naturally occurring amino acid
  VARIANT 44: note = MISC_FEATURE - Xaa can be any naturally occurring amino acid
  VARIANT 50: note = MISC_FEATURE - Xaa is Val or Ile
  VARIANT 51..53: note = MISC_FEATURE - Xaa can be any naturally occurring amino acid
  VARIANT 55..56: note = MISC_FEATURE - Xaa can be any naturally occurring amino acid
  VARIANT 57..58: note = MISC_FEATURE - Xaa can be any naturally occurring amino acid or absent
  VARIANT 59: note = MISC_FEATURE - Xaa can be any naturally occurring amino acid
  VARIANT 60: note = MISC_FEATURE - Xaa is Ala or Ser
  VARIANT 61..63: note = MISC_FEATURE - Xaa can be any naturally occurring amino acid or absent
  VARIANT 64..67: note = MISC_FEATURE - Xaa can be any naturally occurring amino acid
  VARIANT 68: note = MISC_FEATURE - Xaa is Ser or Ala
  VARIANT 69: note = MISC_FEATURE - Xaa can be any naturally occurring amino acid
  VARIANT 71: note = MISC_FEATURE - Xaa can be any naturally occurring amino acid
  VARIANT 72: note = MISC_FEATURE - Xaa is Leu or Val
  VARIANT 73: note = MISC_FEATURE - Xaa can be any naturally occurring amino acid
  VARIANT 74: note = MISC_FEATURE - Xaa is Ala or Ser
  VARIANT 76..77: note = MISC_FEATURE - Xaa can be any naturally occurring amino acid
  VARIANT 79: note = MISC_FEATURE - Xaa can be any naturally occurring amino acid
  VARIANT 81: note = MISC_FEATURE - Xaa can be any naturally occurring amino acid
  VARIANT 84..85: note = MISC_FEATURE - Xaa can be any naturally occurring amino acid
  VARIANT 87: note = MISC_FEATURE - Xaa can be any naturally occurring amino acid
  VARIANT 89..90: note = MISC_FEATURE - Xaa can be any naturally occurring amino acid
  VARIANT 92: note = MISC_FEATURE - Xaa is Glu or Asp
  VARIANT 93: note = MISC_FEATURE - Xaa can be any naturally occurring amino acid
  VARIANT 94: note = MISC_FEATURE - Xaa is Gly or Ala
  VARIANT 97..99: note = MISC_FEATURE - Xaa can be any naturally occurring amino acid
  VARIANT 100: note = MISC_FEATURE - Xaa is Leu of Val
  VARIANT 101..102: note = MISC_FEATURE - Xaa can be any naturally occurring amino acid
  VARIANT 105: note = MISC_FEATURE - Xaa can be any naturally occurring amino acid
  VARIANT 110..111: note = MISC_FEATURE - Xaa can be any naturally occurring amino acid
  VARIANT 113: note = MISC_FEATURE - Xaa can be any naturally occurring amino acid
  VARIANT 115..119: note = MISC_FEATURE - Xaa can be any naturally occurring amino acid
  VARIANT 120: note = MISC_FEATURE - Xaa is Ser or Ala
  VARIANT 121..122: note = MISC_FEATURE - Xaa can be any naturally occurring amino acid
  VARIANT 124..126: note = MISC_FEATURE - Xaa can be any naturally occurring amino acid
  VARIANT 127: note = MISC_FEATURE - Xaa is Lys or Arg
  VARIANT 128..135: note = MISC_FEATURE - Xaa can be any naturally occurring amino acid
  VARIANT 137: note = MISC_FEATURE - Xaa can be any naturally occurring amino acid
  VARIANT 144: note = MISC_FEATURE - Xaa can be any naturally occurring amino acid
  VARIANT 146: note = MISC_FEATURE - Xaa is Val or Ile
  VARIANT 148..149: note = MISC_FEATURE - Xaa can be any naturally occurring amino acid
  VARIANT 152: note = MISC_FEATURE - Xaa can be any naturally occurring amino acid
  VARIANT 154: note = MISC_FEATURE - Xaa can be any naturally occurring amino acid
  VARIANT 157..158: note = MISC_FEATURE - Xaa can be any naturally occurring amino acid
  VARIANT 160: note = MISC_FEATURE - Xaa can be any naturally occurring amino acid
  VARIANT 162: note = MISC_FEATURE - Xaa is Leu or Met
  VARIANT 163..164: note = MISC_FEATURE - Xaa can be any naturally occurring amino acid
  VARIANT 165: note = MISC_FEATURE - Xaa can be any naturally occurring amino acid absent
  VARIANT 168: note = MISC_FEATURE - Xaa can be any naturally occurring amino acid
  VARIANT 173: note = MISC_FEATURE - Xaa can be any naturally occurring amino acid
  VARIANT 175: note = MISC_FEATURE - Xaa can be any naturally occurring amino acid
  VARIANT 177: note = MISC_FEATURE - Xaa is Leu or Val
  VARIANT 179: note = MISC_FEATURE - Xaa can be any naturally occurring amino acid
  VARIANT 182: note = MISC_FEATURE - Xaa can be any naturally occurring amino acid
  VARIANT 185: note = MISC_FEATURE - Xaa can be any naturally occurring amino acid
XSXTPLPXDXXDSGLDDKEDXXCFFEACVEXXXXXEEXXXXXAXPNESAXXXXAXXXXXXXXXXXXXXXSXXXXDXXDXVXLSXXSXQXXAXXXKEXXXXXXTSXGCAEXXEXTXXXXXXXXEXXXXXXXXXXXXEXNAASAKXKXQXXSLXAXQPXXKXAXXXXSPXTLTCXEXLXNXHEXRHXNMHR

SEQ ID NO: 23 - protein, 188 aa, synthetic construct
  REGION 1..188: note = Protein
  VARIANT 2: note = misc_feature - Xaa can be any naturally occurring amino acid
  VARIANT 7: note = misc_feature - Xaa can be any naturally occurring amino acid
  VARIANT 9..10: note = misc_feature - Xaa can be any naturally occurring amino acid
  VARIANT 20..21: note = misc_feature - Xaa can be any naturally occurring amino acid
  VARIANT 30..34: note = misc_feature - Xaa can be any naturally occurring amino acid
  VARIANT 37..41: note = misc_feature - Xaa can be any naturally occurring amino acid
  VARIANT 43: note = misc_feature - Xaa can be any naturally occurring amino acid
  VARIANT 49..52: note = misc_feature - Xaa can be any naturally occurring amino acid
  VARIANT 54..68: note = misc_feature - Xaa can be any naturally occurring amino acid
  VARIANT 70..73: note = misc_feature - Xaa can be any naturally occurring amino acid
  VARIANT 75..76: note = misc_feature - Xaa can be any naturally occurring amino acid
  VARIANT 78: note = misc_feature - Xaa can be any naturally occurring amino acid
  VARIANT 80: note = misc_feature - Xaa can be any naturally occurring amino acid
  VARIANT 83..84: note = misc_feature - Xaa can be any naturally occurring amino acid
  VARIANT 86: note = misc_feature - Xaa can be any naturally occurring amino acid
  VARIANT 88..89: note = misc_feature - Xaa can be any naturally occurring amino acid
  VARIANT 91..93: note = misc_feature - Xaa can be any naturally occurring amino acid
  VARIANT 96..101: note = misc_feature - Xaa can be any naturally occurring amino acid
  VARIANT 104: note = misc_feature - Xaa can be any naturally occurring amino acid
  VARIANT 109..110: note = misc_feature - Xaa can be any naturally occurring amino acid
  VARIANT 112: note = misc_feature - Xaa can be any naturally occurring amino acid
  VARIANT 114..121: note = misc_feature - Xaa can be any naturally occurring amino acid
  VARIANT 123..134: note = misc_feature - Xaa can be any naturally occurring amino acid
  VARIANT 136: note = misc_feature - Xaa can be any naturally occurring amino acid
  VARIANT 143: note = misc_feature - Xaa can be any naturally occurring amino acid
  VARIANT 145: note = misc_feature - Xaa can be any naturally occurring amino acid
  VARIANT 147..148: note = misc_feature - Xaa can be any naturally occurring amino acid
  VARIANT 151: note = misc_feature - Xaa can be any naturally occurring amino acid
  VARIANT 153: note = misc_feature - Xaa can be any naturally occurring amino acid
  VARIANT 156..157: note = misc_feature - Xaa can be any naturally occurring amino acid
  VARIANT 159: note = misc_feature - Xaa can be any naturally occurring amino acid
  VARIANT 161..164: note = misc_feature - Xaa can be any naturally occurring amino acid
  VARIANT 167: note = misc_feature - Xaa can be any naturally occurring amino acid
  VARIANT 172: note = misc_feature - Xaa can be any naturally occurring amino acid
  VARIANT 174: note = misc_feature - Xaa can be any naturally occurring amino acid
  VARIANT 176: note = misc_feature - Xaa can be any naturally occurring amino acid
  VARIANT 178: note = misc_feature - Xaa can be any naturally occurring amino acid
  VARIANT 181: note = misc_feature - Xaa can be any naturally occurring amino acid
  VARIANT 184: note = misc_feature - Xaa can be any naturally occurring amino acid
SXTPLPXDXXDSGLDDKEDXXCFFEACVEXXXXXEEXXXXXAXPNESAXXXXAXXXXXXXXXXXXXXXSXXXXDXXDXVXLSXXSXQXXAXXXKEXXXXXXTSXGCAEXXEXTXXXXXXXXEXXXXXXXXXXXXEXNAASAKXKXQXXSLXAXQPXXKXAXXXXSPXTLTCXEXLXNXHEXRHXNMHR